SEQUENCE LISTING (12 sequences)

SEQ ID NO: 1 - protein, 143 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 2 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 3 - protein, 144 aa, synthetic construct [sequence omitted]

SEQ ID NO: 4 - protein, 140 aa, synthetic construct
MQDPYVKEAENLKKYFNAGHSDVADNGTLFLGILKNWKEESDRKIMQSQIVSFYFKLFKNFKDDQCIQKSVETIKEDMNVKFFNSNKKKRDDFEKLTNYSVTDLNVQRKAIHELIQVMAELSPAAKTGKRKRSQMLFRGR

SEQ ID NO: 5 - protein, 140 aa, synthetic construct
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTCYSVTDLNVQRKAIHELIQVMAELSPAAKTGKRKRSQMLFRGR

SEQ ID NO: 6 - protein, 140 aa, synthetic construct
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQCLFRGR

SEQ ID NO: 7 - protein, 140 aa, synthetic construct
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGR

SEQ ID NO: 8 - protein, 15 aa, Homo sapiens
KTGKRKRSQMLFRGR

SEQ ID NO: 9 - protein, 134 aa, Mus musculus
MHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 10 - protein, 4 aa, Homo sapiens
KRKR

SEQ ID NO: 11 - protein, 7 aa, Homo sapiens
KTGKRKR

SEQ ID NO: 12 - protein, 4 aa, Homo sapiens
RGRR

(19) United States
(12) Patent Application Publication
(10) Pub. No.: US 2025/0270273 A1
(43) Pub. Date: Aug. 28, 2025

(54) POLYMER ENGINEERED FORMS OF INTERFERON-GAMMA AND METHODS OF USE

(71) Applicant: Nektar Therapeutics, San Francisco, CA (US)

(72) Inventors: Deborah H. Charych, Albany, CA (US); Dawei Sheng, Fremont, CA (US); Jicai Huang, San Mateo, CA (US); Wei Chan, San Francisco, CA (US); Damon Justin Hamel, San Bruno, CA (US)

(21) Appl. No.: 18/578,526

(22) PCT Filed: Jul. 12, 2022
(86) PCT No.: PCT/US2022/073649

§ 371 Date: Jan. 11, 2024

Related U.S. Application Data
(60) Provisional application No. 63/319,234, filed on Mar. 11, 2022.
(60) Provisional application No. 63/220,795, filed on Jul. 12, 2021.

(30) Foreign Application Priority Data
Jul. 12, 2021 (US) 63220795
Mar. 11, 2022 (US) 63319234

(51) Int. Cl. C07K 14/57; A61K 38/21; A61K 45/06; A61K 47/60; A61P 35/00

(52) U.S. Cl. CPC C07K 14/57; A61K 38/217; A61K 45/06; A61K 47/60; A61P 35/00

(57) ABSTRACT

Provided is a conjugate comprising interferon-gamma (IFN-gamma) covalently attached to a water-soluble polymer at a cysteine residue of the IFN-gamma, more particularly, at the sulfur atom of the cysteine, where the interferon-gamma is a mutein comprising a cysteine that has been substituted for an amino acid of the IFN-gamma or has been inserted into the IFN-gamma sequence (“cysteine mutein of IFN-gamma”). In preferred embodiments, the water-soluble polymer is a poly(alkylene oxide). More preferably, the water-soluble polymer is a poly(ethylene glycol).

### CROSS REFERENCE TO RELATED APPLICATIONS

This application claims the benefit of priority under 35 U.S.C. § 119(e) to U.S. Provisional Patent Application No. 63/319,234, filed on Mar. 11, 2022 and to U.S. Provisional Patent Application No. 63/220,795, filed on Jul. 12, 2021, the disclosures of which are incorporated herein by reference in their entirety.

### FIELD

The instant disclosure is directed to, among other things, polymer-modified forms of interferon-γ (“IFN-γ”) muteins, related compositions and methods of preparation and use, for example, in the treatment of conditions responsive to therapy with IFN-γ effective to provide sustained immune activation and/or anti-tumor activity.

### BACKGROUND

Interferon-γ (“IFNγ”, “IFN-γ” or “IFN-g”) is a pleiotropic cytokine that is the sole member of the type II interferon class and that mediates both innate and adaptive immune responses. IFN-γ is a 28 kilodalton (kDa) homodimer that signals through binding to the extracellular domain of the heterodimeric cell-surface receptor (IFNγR) composed of an alpha chain (IFNγR1) and a β chain (IFNγR2), which further activates the JAK/STAT pathway (Platanias, et al., Nature Reviews Immunology, 5:375-386 (2005)). Mature human IFN-γ protein contains 143 amino acids per monomer (Fam et al., J Interferon Cytokine Res, 2014, 34(10):759-768). IFN-γ is primarily secreted by activated T cells (tumor-specific cytotoxic CD8+T lymphocytes) and natural killer (NK) cells. Biologically active IFN-γ exists in solution as a non-covalently linked homodimer (Alspach et al., Cold Spring Harb Perspect Biol, doi: 10.1101/cshperspect.ao28480 (2018)).

IFN-γ signaling plays a role in activation of a number of immune cells including, at least, macrophages, antigen-presenting cells, and B cells. Native IFN-γ is essential for Th1 immune responses and regulates T cell differentiation, activation, expansion, homeostasis, and survival (Miller et al., Ann NY Acad Sci, 1182:69-79 (2009)). IFN-γ contributes to the innate immune response by reprogramming macrophages to the M1 proinflammatory phenotype (Jorgovanovic, et al., Biomarker Research, 2020, 8:49:2-16). IFN-γ is the most potent macrophage-activating cytokine and is responsible for rendering macrophage cells capable of increased proinflammatory cytokine synthesis, enhanced phagocytosis, and enhanced antigen presenting capacity (Alspach (2018)). IFN-γ signaling influences the process of antigen processing and presentation. IFN-γ signaling in antigen-presenting cells (APCs) further results in up-regulation of costimulatory molecules and cytokines involved in the production of effective T cell responses (Alspach (2018)). Further, B cell proliferation and antibody class switching are both regulated by IFN-γ (Alspach (2018)). IFN-γ can promote macrophage activation, mediate antiviral and antibacterial immunity, enhance antigen presentation, orchestrate activation of the innate immune system, coordinate lymphocyte-endothelium interaction, regulate Th1/Th2 balance, and control cellular proliferation and apoptosis (Tau et al., Allergy, 54(12):1233-1251 (1999)).

The FDA approved the IFN-γ-1 b protein (SEQ ID NO:7) for treatment of chronic granuloma and to slow osteopetrosis (ACTIMMUNE®, Horizon Pharma). In recent years, the role of IFN-γ as an immunostimulatory agent and its immunomodulatory effects have led to a number of clinical trials investigating the use of IFN-γ-1 b for a number of indications (see clinicaltrials.gov website). Further, IFN-γ may directly cause tumor apoptosis in the absence of immune cells, primarily mediated by Fas (CD95), a type I membrane protein (Ahn et al., Int. J. Cancer, 2002, 100:445-451). The IFN-γ protein may also indirectly cause tumor destruction by activation of innate and adaptive immune responses (Chen et al., Oncoimmunology, 2013, (7):e24964, FIG. 1). In studies of immunotherapeutic agents, an increase in IFN-γ produced by peripheral lymphocytes was indicative of an efficacious response (McNamara et al., Cancer Immunol. Res., 2016, 4:650-657). Although potent in vitro, IFN-γ has provided mixed results at best when investigated in human clinical studies (Reed et al., J. Interferon & Cytokine Res., 2008, 28:611-622; Schroder et al., J. Leukocyte Biol., 2004, 75:163-189). IFN-γ has a reported half-life in humans of about 30 minutes after intravenous injection, about 4.5 hours after intramuscular injection, and about 6 hours after subcutaneous injection. The short in vivo half-life of IFN-γ may limit its clinical application where longer exposure is desired or necessitated. Indeed, ACTIMMUNE®, interferon-γ-1 b, typically requires three separate administrations per week for treating the symptoms of chronic granulomatous disease and malignant osteopetrosis. A longer-acting IFN-γ compound has been described in which polyethylene glycol was covalently attached to IFN-γ, more particularly, to a cysteine that was substituted for leucine-103 (L103C) (see Fam, C. M., et al. (J. Interferon & Cytokine Research, 34(10):759-768, 2014) and U.S. Pat. No. 9,296,804). However, this compound has not been further developed for treating cancer and does not appear to be under clinical investigation.

However, IFN-γ has also been found to play a role in promoting tumor growth and progression (Jorgovanovic (2020)). Recent studies described in Jorgovanovic postulated that the concentration of IFN-γ in the tumor microenvironment (TME) may determine its role in tumor progression and regression. As an example, pretreatment with a low dose of IFN-γ in a murine colon adenocarcinoma model was found to enhance the metastatic potential of the colon 26 tumor cells (Kelly, et al., Cancer Research, 51:4020-4017 (1991)).

Further complicating the therapeutic use of IFN-γ, it has been reported that the biological activity of IFN-γ is inhibited by heparin, a common glycosaminoglycan found in the extracellular matrix of multiple tissue types (Fritchley, Clin. Exp. Immunol., 2000, 120:247-252; Fluhr et al., Fertility and Sterility, 2011, 95(4):1272-1277). In particular, IFN-γ-induced MHC expression, a potentially therapeutic feature of IFN-γ, was compromised in the presence of heparin. It has further been reported that heparin mediates the activities of IFN-γ by binding to the carboxy-terminal (C-terminal) domain of IFN-γ (Lortat-Jacob, et al., J. Biol. Chem., 271(27):16139-16143 (1996)).

Thus, while numerous mouse studies originally suggested an important role for IFN-γ in tumor immunity, IFN-γ has demonstrated limited clinical utility to date (Lee and Margolin, Cancers, 2011, 3(4):3856-3893).

Notwithstanding the foregoing, there remains a need for improved immunotherapies, such as anticancer and other immunotherapies. The present disclosure addresses these and other needs by providing polymer engineered forms of IFN-γ (the conjugates having a number of advantageous features to be described in greater detail below), as well as compositions, and kits comprising such conjugates, and related methods of preparation and use, as described herein, which are believed to be new and completely unsuggested by the art.

### SUMMARY

In a first aspect, provided is a conjugate comprising interferon-γ (IFN-γ) covalently attached to a water-soluble polymer at a cysteine residue of the IFN-γ, more particularly, at the sulfur atom of the cysteine, where the interferon-γ is a mutein comprising a cysteine that has been substituted for an amino acid of the IFN-γ or has been inserted into the IFN-γ sequence (“cysteine mutein of IFN-γ”). In preferred embodiments, the water-soluble polymer is a poly(alkylene oxide). More preferably, the water-soluble polymer is a poly(ethylene glycol).

In some embodiments, the conjugate has a structure:

IFN-γ-s-x-POLY Formula I

where IFN-γ is a cysteine mutein of IFN-γ (as described above), S is a sulfur atom of the cysteine, X is an optional spacer moiety interposed between the cysteine sulfur atom and POLY, and POLY is a water-soluble, non-peptidic polymer moiety. In one or more preferred embodiments, spacer X is present. In some other embodiments, spacer X is absent.

In some further embodiments, the conjugate has a structure:

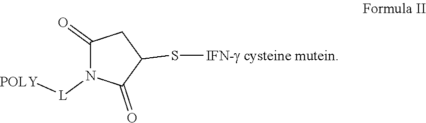

In Formula II, with reference to Formula I, the spacer moiety X comprises

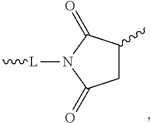

wherein L is a linker comprising one or more atoms selected from carbon, nitrogen, sulfur, oxygen, and combinations thereof, POLY is a water-soluble, non-peptidic polymer; and —S— is a sulfur atom of the cysteine. In such embodiments, the covalent linkage to IFN-γ is via a thioether bond (“—S—”) that is typically formed by reaction of a maleimidyl-functionalized water-soluble polymer, e.g., a maleimidyl functionalized poly(ethylene glycol) reagent, with a thiol-group of a cysteine of the IFN-γ cysteine mutein.

In yet some further embodiments, provided are conjugates having a “ring-open” structure:

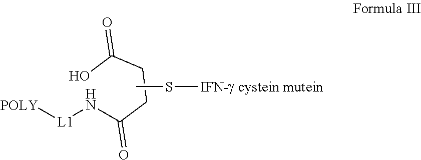

wherein, in reference to Formula I, spacer moiety X comprises

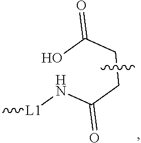

wherein L1 is a linker comprising one or more atoms selected from carbon, nitrogen, sulfur, oxygen, and combinations thereof, POLY is a water-soluble, non-peptidic polymer; and —S— is a sulfur atom of the cysteine. In embodiments related to Formula III, the covalent linkage to IFN-γ is via a thioether bond (“—S—”) that may be formed by reaction of a maleimidyl-functionalized water-soluble polymer, e.g., a maleimidyl functionalized poly(ethylene glycol) reagent, with a thiol-group of a cysteine of the IFN-γ cysteine mutein. In Formula III, with reference to Formula II, it can be seen that the resulting succinimidyl-ring is in its ring-open, hydrolyzed form. In some embodiments, it may be preferred to have the interferon-γ cysteine mutein conjugate in its ring-open form, advantages of which will be described in greater detail below. It will be appreciated that Formula III encompasses structural isomers differing in the point of attachment of the thiol group of the IFN-γ to form the resulting thioether linkage, that is, at either of the carbon atoms positioned between the carboxyl group and the amide group of the succinamic acid.

In one or more additional embodiments related to any one or more of the foregoing formulae, the spacer or linker comprises one or more atoms selected from a carbon-carbon bond, an amide, a carbamate, and an amine, an ether, and combinations thereof. In some more particular embodiments related to any one or more of the foregoing structures or formulae, linker L or L1 is ˜(CH₂)ₐ(O)_{b}[C(O)]_{c}(NH)_{d}(CH₂)ₑ(NH)_{f}[C(O)]_{g}(CH₂)ₕ˜, wherein: a is 0-10; b is 0, 1; c is 0, 1; d is 0, 1; e is 0-10; f is 0, 1; g is 0, 1; and h is 0-10, wherein at least one of a, b, c, d, e, f, g, and h is a positive integer. For example, variable “a” may have a value of 0, 1, 2, 3, 4, 5, 6, 7, 8, 9, or 10. That is, the —CH₂— group may be absent (a=0), or may be one of the following: —(CH₂), —(CH₂)₂, —(CH₂)₃, —(CH₂)₄, —(CH₂)₅, —(CH₂)₆, —(CH₂)₇, —(CH₂)₈, —(CH₂)₉, or —(CH₂)₁₀. In some embodiments, b is zero (such that the oxygen atom is absent. In some other embodiments, b is 1 (such that the oxygen atom is present). Similarly, in some embodiments, c is zero (i.e., is absent). In some other embodiments, c is 1 (i.e., c is present). Similarly, in some embodiments, d is zero (i.e., is absent). In some other embodiments, d is 1 (i.e., d is present). In some embodiments, the linker comprises an amide function such that both c and d are one (or both f and g are one). In some embodiments, variable “e” has a value of 0, 1, 2, 3, 4, 5, 6, 7, 8, 9, or 10. That is, the —CH₂— group may be absent (e=0), or may be one of the following: —(CH₂), —(CH₂)₂, —(CH₂)₃, —(CH₂)₄, —(CH₂)₅, —(CH₂)₆, —(CH₂)₇, —(CH₂)₈, —(CH₂)₉, or —(CH₂)₁₀. In some embodiments, f is 0 (the —NH— group is absent). In some embodiments, f is 1 (the —NH— group is present). In some embodiments, g is 0 (such that the carbonyl carbon is absent); in some embodiments, g is 1 (such that the carbonyl carbon is present). In some embodiments, h is zero (such that the —CH₂— group is absent). In some embodiments, h is a positive integer selected from 1-10, such that the corresponding collection of atoms is —(CH₂), —(CH₂)₂, —(CH₂)₃, —(CH₂)₄, —(CH₂)₅, —(CH₂)₆, —(CH₂)₇, —(CH₂)₈, —(CH₂)₉, or —(CH₂)₁₀. In some particular embodiments, f is 1, g is 1 and h is 2. In yet some further embodiments, a, b, c, d, and e are zero. In one or more further embodiments, a is 3, b is 0, c is 1, d is 1 and e is 2. In yet some additional embodiments, f is 1, g is 1, h is 2, and a, b, c, d, and e are zero. In yet some more particular embodiments of the linker, f is 1, g is 1, h is 2, a is 3, b is 0, c is 1, d is 1 and e is 2.

The water-soluble polymer may have any of a number of different architectures. For example, the water-soluble polymer, POLY, may be linear or branched. In embodiments in which POLY is branched, in some additional embodiments, the branched POLY comprises from about 2 to about 10 polymer arms. In some preferred embodiments, the branched polymer comprises two polymer arms.

The water-soluble polymer, POLY, in some embodiments, is selected from poly(alkylene oxide), poly(vinyl pyrrolidone), poly(vinyl alcohol), polyoxazoline, poly(acryloylmorpholine), polyacrylic acid, polyacrylamides, N-(2-hydroxypropyl) methylacrylamide, divyinyl ether-maleic anhydride, polyphosphates, polyphosphazenes, and co-polymers and ter-polymers thereof. In preferred embodiments, POLY is a poly(alkylene oxide) such as poly(ethylene glycol). In some particular embodiments, POLY comprises —(CH₂CH₂O)ₙ—Y or —(OCH₂CH₂)ₙ—Y′ (depending upon how the repeat unit is considered), wherein Y is selected from a lower alkyl (e.g., methyl) or hydrogen, and Y′ is selected from a lower alkoxy (e.g., methoxy) and hydroxyl, and has a weight average molecule weight in a range of from about 200 daltons to about 80,000 daltons or more, where n is an integer having an average value of from about 5 to about 2,000. In some further embodiments, n is an integer having an average value ranging from about 45 to about 1818.

In some embodiments, POLY has a weight average molecular weight of from about 2,000 daltons to about 80,000 daltons; in some other embodiments, POLY has a weight average molecular weight of from about 2,000 daltons to about 40,000 daltons; in yet some additional embodiments, POLY has a weight average molecular weight of from about 10,000 daltons to about 40,000 daltons. Exemplary weight average molecular weights of POLY include 2,000 daltons, 5,000 daltons, 10,000 daltons, 15,000 daltons, 20,000 daltons, 25,000 daltons, 30,000 daltons, 35,000 daltons, 40,000 daltons, 45,000 daltons, 50,000 daltons, 55,000 daltons, 60,000 daltons, and higher. Further molecular weights are also contemplated.

In embodiments in which POLY is branched and has two polymer arms, in some further embodiments, POLY comprises a structure:

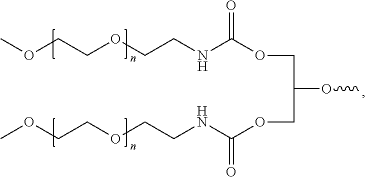

where each n independently falls within a range as described above or elsewhere herein or has a value as described above or elsewhere herein. In some preferred embodiments, the value of “n” in each polymer arm is approximately the same.

In turning to the engineered IFN-γ molecule, in some further embodiments, the IFN-γ cysteine mutein has a sequence having at least 95% sequence identity to a sequence as set forth in SEQ ID NO:1, SEQ ID NO:2, SEQ ID NO:3, or SEQ ID NO:7 and comprises the cysteine substitution or insertion. In some particular embodiments, the IFN-γ cysteine mutein has a sequence set forth in SEQ ID NO: 4, SEQ ID NO: 5, or SEQ ID NO:6, In some further embodiments, the IFN-γ cysteine mutein sequence includes a cysteine residue substituted for at least one amino acid selected from the group consisting of S66, N98, and M135 of SEQ ID NO:3 or SEQ ID NO:7. In some preferred embodiments, the IFN-γ cysteine mutein sequence includes a cysteine residue substituted for M135 of SEQ ID NO:3 or SEQ ID NO:7. In yet some further embodiments which may be preferred, the cysteine substitution or insertion is located within the interferon gamma receptor 1 (IFNGR1) binding region of the IFN-γ protein or is located within from one to ten amino acids from either end of the IFNGR1 binding region of the IFN-γ mutein. In yet some further embodiments, the cysteine substitution or insertion is located at the C-terminus of the IFN-γ.

In some particular embodiments, the conjugate has a structure:

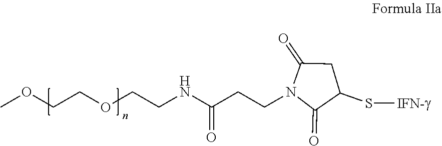

wherein IFN-γ is a cysteine mutein of IFN-γ, n is an integer from about 45 to about 1818, preferably from about 113 to about 1818, and —S— is a sulfur atom of the cysteine. Preferably in some embodiments, n is an integer having a value that corresponds to a polyethylene glycol polymer having a weight average molecular weight selected from about 2,000 daltons (n is ˜45), 5,000 daltons (n is ˜113), 10,000 daltons (n is ˜227), 15,000 daltons (n is ˜340), 20,000 daltons (n is ˜454), 25,000 daltons (n is ˜568), 30,000 daltons (n is ˜681), 40,000 daltons (n is ˜909), 50,000 daltons (n is ˜1136), 60,000 daltons (n is ˜1364), and 80,000 daltons (n is ˜1818). In yet some additional embodiments, the conjugate is in a ring-open form of Formula IIa (to be described in greater detail below).

In yet one or more further embodiments, the conjugate has a structure:

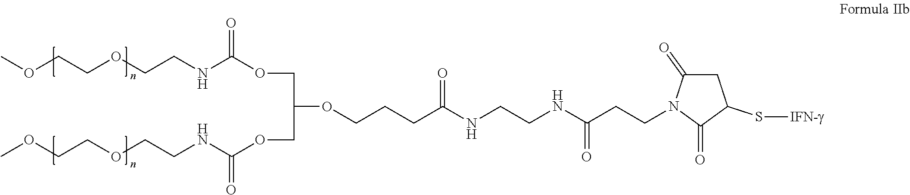

wherein IFN-γ is a cysteine mutein of IFN-γ, each n for Formula IIb is independently an integer from about 45 to about 909, preferably from about 113 to about 909, and —S— is a sulfur atom of the cysteine. In some preferable embodiments, n in each instance is independently an integer having a value that corresponds to a polyethylene glycol polymer chain having a weight average molecular weight selected from the group consisting of 2,000 daltons (n is ˜45), 5,000 daltons (n is ˜113), 10,000 daltons (n is ˜227), 15,000 daltons (n is ˜340), 20,000 daltons (n is ˜454), 25,000 daltons (n is ˜568), 30,000 daltons (n is ˜681), or 40,000 daltons (n is ˜909). In some preferred embodiments, the weight average molecular weight in each poly(ethylene glycol) chain of a branched polymer having a structure as shown in Formula IIb is approximately the same. In yet some additional embodiments, the conjugate is in a ring-open form of Formula IIb (to be described in greater detail below).

In yet one or more further embodiments, the conjugate has a structure:

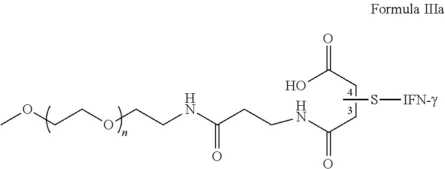

wherein IFN-γ is a cysteine mutein of IFN-γ, n is an integer from about 45 to about 1818, preferably from about 113 to about 1818, and —S— is a sulfur atom of the cysteine. Preferably in some embodiments, n is an integer having a value that corresponds to a polyethylene glycol polymer chain having a weight average molecular weight selected from the group consisting of about 2,000 daltons (n is ˜45), 5,000 daltons (n is ˜113), 10,000 daltons (n is ˜227), 15,000 daltons (n is ˜340), 20,000 daltons (n is ˜454), 25,000 daltons (n is ˜568), 30,000 daltons (n is ˜681), 40,000 daltons (n is ˜909), 50,000 daltons (n is ˜1136), 60,000 daltons (n is ˜1364), and 80,000 daltons (n is ˜1818).

In yet some even further embodiments, the conjugate has a structure:

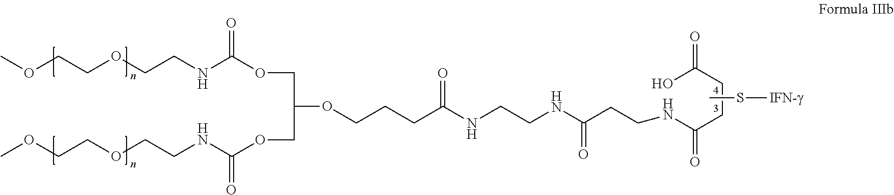

wherein IFN-γ is a cysteine mutein IFN-γ, n for Formula IIIb is independently an integer from about 45 to about 909, preferably from about 113 to about 909, and —S— is a sulfur atom of the cysteine. In some preferable embodiments, each n is an integer having a value that corresponds to a polyethylene glycol polymer chain having a weight average molecular weight selected from the group consisting of about 2,000 daltons (n is ˜45), 5,000 daltons (n is ˜113), 10,000 daltons (n is ˜227), 15,000 daltons (n is ˜340), 20,000 daltons (n is ˜454), 25,000 daltons (n is ˜568), 30,000 daltons (n is ˜681), or 40,000 daltons (n is ˜909). It will be appreciated that Formulae III, IIIa, and IIIb encompass structural isomers differing in the point of attachment to the cysteine sulfur atom, that is, at the 3-position or the 4-position of the succinamic acid as shown above.

In some specific embodiments, the conjugate has a structure selected from:

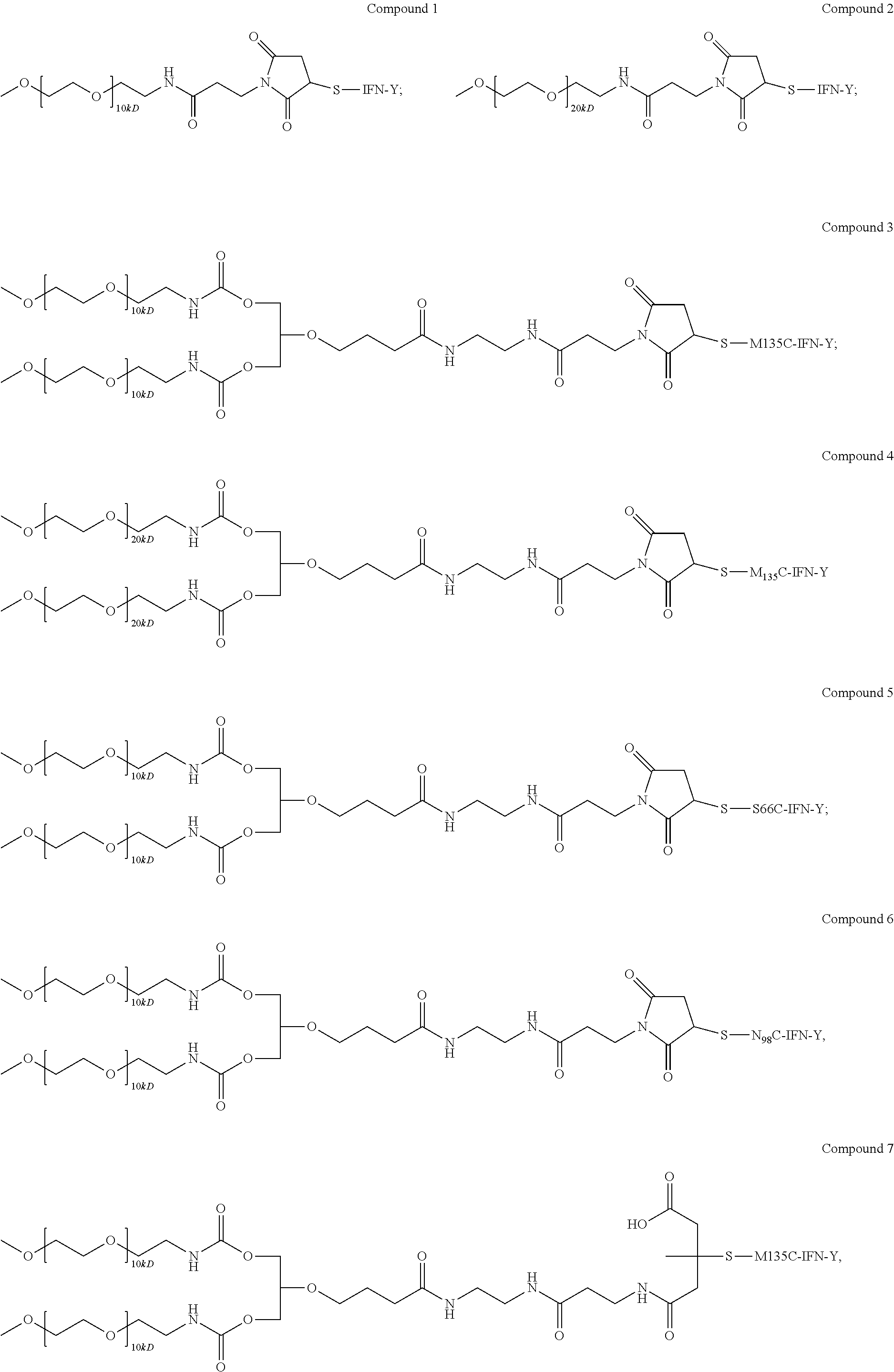

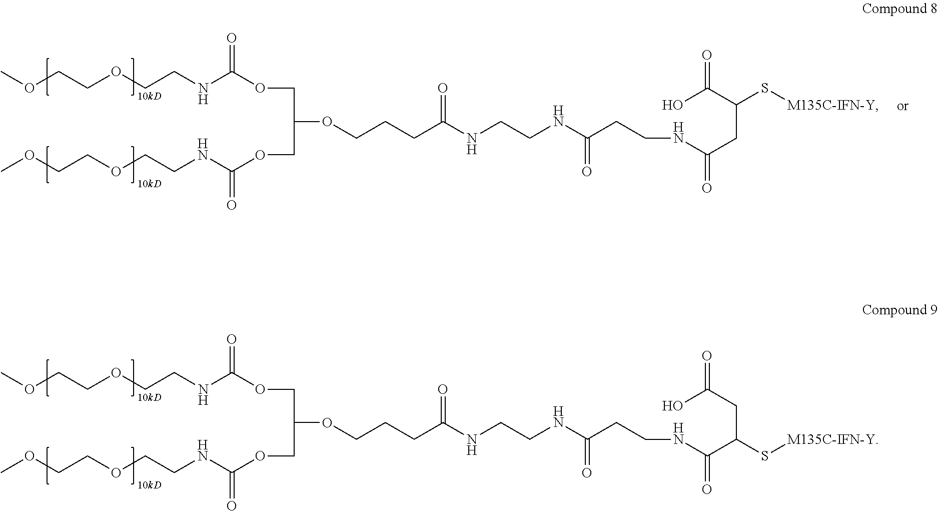

In some embodiments, the water-soluble polymer is attached to a cysteine that has been substituted for at least one amino acid within the C-terminal region of the IFN-γ protein. The position of substitution is designated by the original amino acid, the position, and the substituted amino acid. Thus, in an exemplary embodiment, cysteine substitution M135C represents substitution of a cysteine for the methionine at position 135 of a particularly referenced sequence. In certain embodiments, the water-soluble polymer is covalently attached to a cysteine that has been substituted or inserted within the heparin binding region of the IFN-γ molecule. In some specific embodiments, the water-soluble polymer is covalently attached to a cysteine that is located within 1-10 amino acids at either end of the heparin binding region of the IFN-γ mutein.

In additional embodiments, the water-soluble polymer is covalently attached to a cysteine located within the interferon gamma receptor 1 (IFNGR1) binding region of the IFN-γ mutein. In yet one or more further embodiments, the water-soluble polymer is covalently attached to a cysteine that is located within about 1-10 amino acids from either end of the IFNGR1 binding region of the IFN-γ mutein. Without being bound by theory, it is believed that in some instances, having the water-soluble polymer attached within the IFNGR1 receptor binding region or heparin binding region, or proximal thereto, may provide a conjugate, i.e., an IFN-γ receptor agonist, that exhibits diminished potency and/or receptor binding (that is, a lower affinity receptor binding) relative to native IFN-γ. Such features may be engineered into a conjugate by, for example, optimal design of the cysteine insertion or substitution site, to thereby allow introduction of a water-soluble polymer moiety at a specific site within the IFN-γ protein molecule. The design of a conjugate having features such as reduced potency and/or receptor binding, particularly when evaluated in vitro, may be better understood by considering that such features may be offset by improved pharmacokinetics and/or improved pharmacodynamics (relative to native IFN-γ), wherein the balance of such features may provide conjugates having one or more advantages over native IFN-γ when used in a clinical setting, such as reduced acute toxicity, prolonged exposure at therapeutically effective levels (enhanced therapeutic index), an improved safety profile by virtue of slower receptor-mediated clearance, resistance to heparin binding, and a prolonged circulating half-life, among other things.

Considering further the IFN-γ molecule engineered to incorporate an accessible cysteine residue, in some embodiments, the IFN-γ mutein has a sequence having at least 95% sequence identity to a sequence set forth in SEQ ID NO: 4, SEQ ID NO: 5, or SEQ ID NO:6 and comprises the cysteine substitution or insertion. In further embodiments, the IFN-γ mutein has a sequence having at least 95% sequence identity to SEQ ID NO: 7, wherein a cysteine residue is substituted for at least one amino acid selected from the group consisting of S66, N98, and M135.

In yet some further embodiments, with reference to the potency of an exemplary IFN-γ mutein conjugate as provided herein, the conjugate has an EC50 value (ng/mL, human PMBCs pSTAT1) that is increased by at least about 3-fold when compared to the EC50 value (ng/mL, human PMBCs pSTAT1) of the non-polymer modified IFN-γ cysteine mutein and/or unmodified IFN-γ, or at least about 3.5-fold, or at least about 4-fold, or at least about 4.5-fold, or at least about 5-fold, or at least about 5.5-fold, or at least about 6-fold, or at least about 6.5-fold, or at least about 7-fold, or at least about 7.5-fold, or at least about 8-fold, or at least about 8.5-fold, or at least about 9-fold, or at least about 9.5-fold, or at least about 10-fold when compared to the EC50 value (ng/mL, human PMBCs pSTAT1) of the non-polymer modified IFN-γ cysteine mutein and/or unmodified IFN-γ.

In next turning to additional features of the present conjugates, in one or more embodiments, the conjugate exhibits a reduction in major histocompatibility complex class I (MHCI) induction of no more than about 3-fold as measured by its EC50 value (ng/mL, HT-29 MHCI) when compared to the MHCI induction as measured by EC50 value (ng/mL, HT-29 MHCI) of the non-polymer modified IFN-γ cysteine mutein and/or unmodified IFN-γ, or no more than about 3.5-fold, or no more than about 4-fold, or no more than about 4.5-fold, or no more than about 5-fold when compared to the MHCI induction as measured by EC50 value (ng/mL, HT-29 MHCI) of the non-polymer modified IFN-γ cysteine mutein and/or unmodified IFN-γ.

Further related to the foregoing, in yet some additional embodiments, a conjugate as provided herein exhibits a reduction in IFNGR1 binding (K_{D}) of at least about 3% when compared to IFNGR1 binding (K_{D}) of the non-polymer modified IFN-γ cysteine mutein and/or unmodified IFN-γ, or at least about 4%, or at least about 5%, or at least about 6%, or at least about 7%, or at least about 8%, or at least about 9%, or at least about 10%.

In yet some additional particular embodiments, a conjugate as provided herein exhibits a decrease in heparin binding (Kᵢ) of at least about 1% when compared to the heparin binding (Kᵢ) of the non-polymer modified IFN-γ cysteine mutein and/or unmodified IFN-γ, or at least about 2%, or at least about 3%, or at least about 4%, or at least about 5% when compared to the heparin binding (Kᵢ) of the non-polymer modified IFN-γ cysteine mutein and/or unmodified IFN-γ.

In yet some further particular embodiments, a conjugate as provided herein exhibits a reduction in heparin binding (Kᵢ, nM) of at least about a 1-fold, or at least about 1.5-fold, when compared to the heparin binding (Kᵢ, nM) of the non-polymer modified IFN-γ cysteine mutein and/or unmodified IFN-γ. Thus, in one or more embodiments, conjugates as provided herein have improved resistance to heparin binding when compared to their non-polymer-modified counterpart molecules (i.e., non-polymer modified IFN-γ cysteine mutein and/or unmodified IFN-γ).

In a second aspect, provided is a pharmaceutical composition comprising a conjugates of a cysteine mutein of interferon-γ (IFN-γ) as described herein and at least one pharmaceutically acceptable excipient.

In some embodiments related to the second aspect, the composition comprises ring-opened conjugates as described generally by Formula III above, including general and particular embodiments thereof,

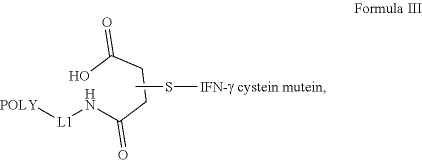

wherein POLY, L1, and IFN-γ cysteine are as previously described (including embodiments thereof), wherein no more than about 15 mole percent of conjugates comprised in the composition have a ring-closed structure:

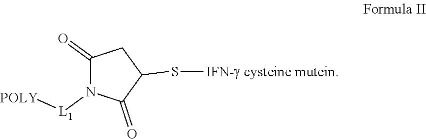

In yet another aspect, provided is a method for treating a subject having a disease or condition that is responsive to treatment with IFN-γ comprising administering to the subject a therapeutically effective amount of an IFN-γ mutein conjugate or composition as described herein, including all embodiments thereof, unless otherwise indicated. In some embodiments, the disease is a cancer. In some further embodiments, the cancer is a liquid cancer. In yet some further embodiments, the cancer is a solid cancer. In yet additional embodiments, the cancer is selected from but not limited to small cell lung cancer (SCLC), non-small cell lung cancer (NSCLC), adrenocortical carcinoma, myxoid/round cell liposarcoma, synovial sarcoma, gliosarcoma, fallopian tube cancer, ovarian cancer, renal cell carcinoma (RCC), colorectal cancer, microsatellite instability-high cancers (MSI-H/dMMR), primary peritoneal cancer, breast cancer, Hodgkin lymphoma, gastric cancer, cervical cancer, primary mediastinal B-cell lymphoma (PMBCL), hepatocellular carcinoma (HCC), Merkel cell carcinoma (MCC), esophageal squamous cell cancer, cutaneous squamous cell carcinoma (cSCC), head and neck squamous cell cancer (HNSCC), bladder cancer, urothelial carcinoma, glioblastoma, melanoma, and T cell lymphomas.

In yet some additional related embodiments, the administering is parenteral. In yet some further related embodiments, the administering step is selected from subcutaneous, intravenous, intra-arterial, intraperitoneal, intracardiac, and intrathecal administration, intramuscular injection, and infusion.

In yet a further aspect, provided is use of an IFN-γ mutein conjugate or composition as disclosed herein for treating a disease or condition that is responsive to treatment with IFN-γ.

In yet a further aspect, provided is use of an IFN-γ mutein conjugate or composition as disclosed herein in the preparation of a medicament for treating a condition that is responsive to treatment with IFN-γ as described elsewhere herein.

In yet another aspect, provided is a combination for use in treating a condition that is responsive to treatment with interferon-γ (IFN-γ), such as e.g., cancer, the combination comprising an IFN-γ mutein conjugate or composition as disclosed herein and one or more of a programmed cell death protein 1 (PD-1) antagonist or a programmed cell death ligand 1 (PD-L1) antagonist.

In yet a further aspect, provided is a method for reducing heparin binding to interferon-γ (IFN-γ) by preparing an IFN-γ cysteine mutein conjugate as described herein.

In yet another aspect, provided is a method for reducing IFN-γ receptor-1 (IFNGR1) binding of an interferon-γ (IFN-γ) by preparing an IFN-γ cysteine mutein conjugate as described herein.

Additional aspects and embodiments are set forth in the following description and claims.

### BRIEF DESCRIPTION OF THE DRAWINGS

FIG. 1 provides the amino acid sequences of native human mature IFN-γ (hIFN-γ) protein (FIG. 1A, SEQ ID NO:1); native hIFN-γ protein with the signal sequence underlined (FIG. 1B, SEQ ID NO:2); hIFN-γ with an included N-terminal methionine for translation initiation (FIG. 1C, SEQ ID NO:3); the S66C mutein of hIFN-γ-1 b (FIG. 1D, SEQ ID NO:4); the N98C mutein of hIFN-γ-1b (FIG. 1E, SEQ ID NO:5); the M135C mutein of hIFN-γ-1b (FIG. 1F, SEQ ID NO:6); the sequence of the hIFN-γ-1b protein (FIG. 1G; SEQ ID NO:7); and the sequence of the mouse (Mus musculus) IFN-γ (mIFN-γ) sequence (FIG. 1H; SEQ ID NO:9).

FIG. 2 is an image of a Coomassie blue stain of purified hIFN-γ-M135C protein as described in Example 1.

FIG. 3 is a LC-ESI-MS chromatogram showing the deconvoluted intact mass spectra for hIFN-γ-S66C (bottom panel), hIFN-γ-N98C (middle panel), and hIFN-γ-M135C (top panel) muteins as detailed in Example 2.

FIG. 4A is the MS/MS fragmentation spectrum for IFN-γ-1 b-S66C peptide 63-69; FIG. 4B is the MS/MS fragmentation spectrum for IFN-γ-1 b-N98C peptide 96-109; and FIG. 4C is the MS/MS fragmentation spectrum for IFN-γ-1b-M135C peptide 132-140 as detailed in Example 3.

FIG. 5 is an image of an SDS-PAGE gel showing a purified mono-PEGylated rhIFN-γ-1b-M135C conjugate, Compound 4, as detailed in Example 4.

FIG. 6 is a graph showing a reverse phase-HPLC chromatogram analysis of purified mono-mPEG2-MAL-20K-rhIFN-γ-1 b-M135C conjugate, Compound 3, as detailed in Example 5.

FIG. 7 is an image of an SDS-PAGE gel showing a purified mono-mPEG2-MAL-20K-rhIFN-γ-1 b-M135C conjugate, Compound 3, as detailed in Example 5.

FIG. 8 is a graph showing a reverse phase-HPLC chromatogram analysis of purified mono-mPEG-MAL-20K-rhIFN-γ-1 b-M135C conjugate, Compound 2, as detailed in Example 6.

FIG. 9 is an image of an SDS-PAGE gel showing a purified mono-mPEG-MAL-20K-rhIFN-γ-1b-M135C conjugate, Compound 2, as detailed in Example 6.

FIG. 10 is a graph showing RP-HPLC chromatogram analysis of purified mono-mPEG-MAL-10K-rhIFN-γ-1b-M135C conjugate, Compound 1, as detailed in Example 7.

FIG. 11 is an image of an SDS-PAGE gel showing a purified mono-mPEG-MAL-10K-rhIFN-γ-1b-M135C conjugate, Compound 1, as detailed in Example 7.

FIG. 12 is a graph showing a RP-HPLC chromatogram analysis of purified mono-mPEG2-MAL-20K-rhIFN-γ-1 b-N98C, Compound 6, as detailed in Example 8.

FIG. 13 is an image of an SDS-PAGE gel showing a purified mono-mPEG2-MAL-20K-rhIFN-γ-1b-N98C conjugate, Compound 6, as detailed in Example 8.

FIG. 14 is a graph of a RP-HPLC chromatogram analysis of purified mono-mPEG2-MAL-20K-rhIFN-γ-1 b-S66C conjugate, Compound 5, as detailed in Example 9.

FIG. 15 is an image of an SDS-PAGE gel showing a purified mono-mPEG2-MAL-20K-rhIFN-γ-1b-S66C conjugate, Compound 5, as detailed in Example 9.

FIG. 16 is a graph of plasma concentration (ng/mL) over time (hours) for rhIFN-γ (●) and Compound 4 (▪) as detailed in Example 15.

FIG. 17A is a graph of the STAT1 signaling in HT-29 cells as background-subtracted homogeneous time resolved fluorescence (HTRF) signal ratio over the Log([TA]) in ng/mL for IFN-γ (∘), Compound 1 (□), Compound 2 (▴), Compound 3 (●), and Compound 4 (▪) as detailed in Example 17. FIG. 17B is a graph of the downstream surface expression of MHCI molecules in HT-29 tumor cells showing the Mean Fluorescence Intensity (MFI) over Log([TA], ng/mL) for IFN-γ (∘), Compound 1 (□), Compound 2 (▴), Compound 3 (●), and Compound 4 (▪) as detailed in Example 17.

FIG. 18A is a graph showing the median HLA-ABC MFI at 24, 72 and 168 hours post administration of a dose of vehicle (●), rhIFN-γ (♦), Compound 4 as a single high dose (▴), and Compound 4 as a single low dose (▾) as detailed in Example 18. FIG. 18B is a graph showing the median HLA-DR/DP/DQ MFI 24, 72 and 168 hours post administration of a dose of vehicle (●), rhIFN-γ (♦), Compound 4 as a single high dose (▴), and Compound 4 as a single low dose (∇) as detailed in Example 18. FIG. 18C is a graph showing the median PD-L1 MFI at 24, 72 and 168 hours post administration of a dose of vehicle (●), rhIFN-γ (♦), Compound 4 as a single high dose (▴), and Compound 4 as a single low dose (∇) as detailed in Example 18.

FIG. 19A is a graph showing the median HLA-ABC mean fluorescence intensity (MFI) at 24, 72 and 168 hours post administration of a dose of vehicle (●), Compound 3, 0.03 mg/kg (♦), Compound 3, 0.003 mg/kg (⋄), Compound 2, 0.03 mg/kg (▾), Compound 2, 0.003 mg/kg (∇), Compound 1, 0.03 mg/kg (▪), and Compound 1, 0.003 mg/kg (□) as detailed in Example 18. FIG. 19B is a graph showing the median HLA-DR/DP/DQ MFI at 24, 72 and 168 hours post administration of a dose of vehicle (●), Compound 3, 0.03 mg/kg (♦), Compound 3, 0.003 mg/kg (⋄), Compound 2, 0.03 mg/kg (▾), Compound 2, 0.003 mg/kg (∇), Compound 1, 0.03 mg/kg (▪), and Compound 1, 0.003 mg/kg (□) as detailed in Example 18. FIG. 19C is a graph showing the median PD-L1 MFI at 24, 72 and 168 hours post administration of a dose of vehicle (●), Compound 3, 0.03 mg/kg (♦), Compound 3, 0.003 mg/kg (⋄), Compound 2, 0.03 mg/kg (▾), Compound 2, 0.003 mg/kg (∇), Compound 1, 0.03 mg/kg (▪), and Compound 1, 0.003 mg/kg (□) as detailed in Example 18.

FIGS. 20A-20C are graphs of competition binding assays of the binding of rhIFN-γ-M135C (FIG. 20A), Compound 1 (FIG. 20B), and Compound 2 (FIG. 20C) to IFNγR1 in the presence of increasing concentrations of heparin as detailed in Example 24.

FIG. 21 is a graph showing a reverse phase-HPLC chromatogram analysis of purified ring-opened mono-mPEG2-MAL-20K-rhIFN-γ-1 b-M135C conjugate, Compound 7, as detailed in Example 10.

FIG. 22 is an image of an SDS-PAGE gel showing a purified ring-opened mono-PEGylated rhIFN-γ-1b-M135C conjugate, Compound 7, as detailed in Example 10.

FIG. 23 is a graph showing a reverse phase-HPLC chromatogram analysis of ring-opened mono-mPEG2-MAL-20K-rhIFN-γ-1 b-M135C conjugate after further purification, Compound 7, as detailed in Example 11.

FIG. 24 is an image of an SDS-PAGE gel showing a ring-opened mono-PEGylated rhIFN-γ-1b-M135C conjugate, Compound 7, after further purification as detailed in Example 11.

FIG. 25 is a graph showing the results of Strong-Cation Exchange (SCX) Chromatography showing the ring-closed (RC) and ring open (RO) forms of mPEG2-MAL-20K-rhIFN-γ-1 b-M135C after incubation of the RC form at pH 8.5 as described in Example 13.

FIG. 26 is a graph showing the deconvoluted intact mass spectrum for mPEG2-MAL-20K-rhIFN-γ-1b-M135C RO Compound 7 as described in Example 14.

FIGS. 27A-27C are graphs showing the median mean fluorescence intensity (MFI) for MHCI (FIG. 27A), MHCII (FIG. 27B) or PD-L1 (FIG. 27C) expression on tumor cells at 1, 3 and 7 days post administration of a dose of vehicle (●), rmIFN-γ, middle dose (▪), Compound 10, high dose (▴), Compound 10, middle dose (▾), and Compound 10, low dose (♦) as described in Example 20.

FIG. 28A is a graph showing tumor infiltrating T cell counts (cells/mm³ of tumor) at 1, 3 and 7 days post administration of a dose of vehicle (●), rmIFN-γ, middle dose, 0.3 mg/kg (▪), Compound 10, high dose, 0.6 mg/kg (▴), Compound 10, middle dose, 0.3 mg/kg (▾), and Compound 10, low dose, 0.1 mg/kg (♦) as described in Example 20. FIG. 28B is a graph showing the percentage of CD8+ T cells in blood (% of CD8+ T cells) at 1, 3 and 7 days post administration of a dose of vehicle (●), rmIFN-γ, middle dose, 0.3 mg/kg (▪), Compound 10, high dose, 0.6 mg/kg (▴), Compound 10, middle dose, 0.3 mg/kg (▾), and Compound 10, low dose, 0.1 mg/kg (♦) as described in Example 20.

### DETAILED DESCRIPTION

Before describing one or more aspects or embodiments of the present disclosure in detail, it should be noted that the present disclosure is not intended to be limited to the particular organic synthetic techniques, IFN-γ moieties, assays, and the like, as such may vary as would be understood by one having ordinary skill in the art to which this disclosure applies.

In describing and claiming certain features of this disclosure, the following terminology will be used in accordance with the definitions described below unless indicated otherwise.

As used in this specification, the singular forms “a,” “an,” and “the” include plural referents unless the context clearly dictates otherwise.

The term “interferon-γ” or “IFN-γ”, as used herein, refers to a polypeptide or protein having human IFN-γ activity. The IFN-γ as referenced for use herein is an “IFN-γ cysteine mutein” (also “IFN-γ mutein”) modified from the natural protein or polypeptide deliberately, as for example, by site directed mutagenesis to include at least one cysteine amino acid. Exemplary IFN-γ muteins comprise an amino acid sequence corresponding to at least one of SEQ ID NOs:4-6, as well as any protein or polypeptide substantially homologous thereto. In embodiments, exemplary IFN-γ muteins comprise an amino acid sequence having at least 90%, 95%, or 99% homology to at least one of SEQ ID NOs:4-6. Further, “IFN-γ” as used herein may refer to either of IFN-γ in dimer form or as a monomer unless otherwise apparent by context. The terms “IFN-γ” and “IFN-γ-1b” are used interchangeably herein unless otherwise apparent by context. In addition, the term “IFN-γ” encompasses both the IFN-γ polypeptide or protein prior to conjugation as well as the IFN-γ polypeptide or protein following conjugation. As will be explained in further detail below, one of ordinary skill in the art can determine whether any given protein or polypeptide has IFN-γ activity. Exemplary, but non-limiting, methods of determining whether a protein or polypeptide has IFN-γ activity are described in Examples 16-17 and 22. It will also be understood that when IFN-γ is attached to a water-soluble polymer such as a polyethylene glycol moiety, IFN-γ is slightly altered due to the presence of one or more covalent bonds associated with linkage to the polymer(s). Reference to an IFN-γ conjugate as described herein is meant to encompass pharmaceutically acceptable salt forms thereof. A “cysteine mutein of IFN-γ” refers to an IFN-γ polypeptide or protein having one or more cysteine insertions and/or substitutions. An insertion refers to insertion of a cysteine amino acid between two naturally occurring amino acids. A substitution refers to substitution of a cysteine amino acid for a naturally occurring amino acid. The position of substitution is typically designated by the original amino acid, the position, and the substituted amino acid. Thus, in an exemplary embodiment, the cysteine substitution M135C represents substitution of a cysteine for the methionine at position 135 of a specifically referenced sequence.

The terms “substantially homologous” or “substantially identical” mean that a particular subject sequence, for example, a mutant sequence, varies from a reference sequence by one or more substitutions, deletions, or additions. For purposes herein, a sequence having greater than 95 percent homology (identity) and equivalent expression characteristics to a given sequence is considered to be substantially homologous (identical). For purposes of determining homology, truncation of the mature sequence should be disregarded. Exemplary IFN-γ peptides or proteins for use herein include those sequences that are substantially homologous to SEQ ID NOs:4-6. It will be appreciated in the instance of an IFN-γ mutein as described herein, substantially homologous sequences include the cysteine mutation as recited herein.

The term “fragment” means any protein or polypeptide having the amino acid sequence of a portion or fragment of an IFN-γ protein or polypeptide, and having the biological activity, or substantially the biological activity, of IFN-γ. Fragments include proteins or polypeptides produced by proteolytic degradation of an IFN-γ moiety as well as proteins or polypeptides produced by chemical synthesis by methods routine in the art.

“Water-soluble, non-peptidic polymer” or “water-soluble polymer” refers to a polymer that is at least 35% (by weight) soluble, preferably greater than 70% (by weight), and more preferably greater than 95% (by weight) soluble, in water at room temperature. Typically, an unfiltered aqueous preparation of a “water-soluble” polymer transmits at least 75%, more preferably at least 95%, of the amount of light transmitted by the same solution after filtering. It is most preferred, however, that the water-soluble polymer is at least 95% (by weight) soluble in water or completely soluble in water. With respect to being “non-peptidic,” a polymer is non-peptidic when it contains less than 35% (by weight) of amino acid residues. An exemplary water-soluble/water-soluble, non-peptidic polymer is a poly(alkylene oxide) such as poly(ethylene glycol).

“PEG”, “polyethylene glycol”, or “poly(ethylene glycol),” as used herein, is meant to encompass any water-soluble poly(ethylene oxide). Unless otherwise indicated, a “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers for use in the present disclosure will comprise one of the two following structures: “—(CH₂CH₂O)ₙ—” or “—(CH₂CH₂O)ₙ₋₁CH₂CH₂—,” depending upon whether or not the terminal oxygen(s) has been displaced, e.g., during a synthetic transformation. For PEG polymers, the variable (n) may range from an average value of from about 5 to about 2,000, or from about 45 to about 1818, or from about 113 to about 1818, and the terminal groups and architecture of the overall PEG can vary. It will be appreciated that where the PEG polymer is branched, the variable (n) for each polymer chain may independently fall within one of the ranges described above or elsewhere herein. Exemplary or preferred PEG-comprising molecules may however comprise one or more particular PEG architectures and/or linkers, and/or molecular weight ranges. PEG polymers in connection with the present disclosure are typically end-capped or terminally capped. Where specific reference is made to PEG hereafter as the water-soluble, non-peptidic polymer, it will be understood that the disclosure relates generally to any water-soluble, non-peptidic polymer or poly(alkylene glycol) with poly(ethylene glycol) being preferred.

The terms “end-capped” and “terminally capped” are interchangeably used herein to refer to a terminal or endpoint of a polymer having a relatively inert end-capping moiety. Typically, although not necessarily, the end-capping moiety comprises a hydroxy group, a lower alkyl group (e.g. a C₁₋₁₀ alkyl group) or a lower alkoxy group (e.g. a C₁₋₁₀ alkoxy group), more preferably a C₁₋₅ alkyl or alkoxy group. Examples of alkoxy end-capping moieties include methoxy and ethoxy. When the polymer is PEG, for example, it is preferred to use a methyl-PEG or methoxy-PEG (commonly referred to as mPEG), in which one terminus of the polymer is a methoxy (—OCH₃) group or a methyl (—CH₃) group.

Molecular weight in the context of a water-soluble polymer, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques (e.g. gel filtration chromatography). Most commonly employed methods are gel permeation chromatography and gel filtration chromatography. Other methods for determining molecular weight include end-group analysis or the measurement of colligative properties (e.g., freezing-point depression, boiling-point elevation, or osmotic pressure) to determine number average molecular weight or the use of light scattering techniques, ultracentrifugation, MALDI TOF, or viscometry to determine weight average molecular weight. PEG polymers are typically polydisperse (i.e., the number average molecular weight and the weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

“Branched,” in reference to the geometry or overall structure of a polymer, refers to a polymer having two or more polymer “arms” or “chains” extending from a branch point. In some preferred embodiments, a branched polymer such as a branched polyethylene glycol possesses two polymer “arms” or “chains” extending from a branch point. Examples of branched polymers are those having two polymer arms comprised of polymer chains having the same structure (for example, comprised of the same monomer subunits), and/or comprised of polymer arms having the same average molecular weight.

A “branch point” refers to a bifurcation point comprising one or more atoms at which a polymer branches or forks from a linear structure into one or more additional arms.

The term “reactive” or “activated” refers to a functional group that reacts readily or at a practical rate under conventional conditions of organic synthesis. This is in contrast to those groups that either do not react or require strong catalysts or impractical reaction conditions in order to react (i.e., a “nonreactive” or “inert” group).

“Not readily reactive,” with reference to a functional group present on a molecule in a reaction mixture, indicates that the group remains largely intact under conditions that are effective to produce a desired reaction in the reaction mixture.

A “releasable linkage” is a relatively labile bond that cleaves under physiological conditions, wherein the cleavage may occur by way of any of a number of different mechanisms. One type of exemplary releasable linkage is a hydrolyzable bond, that is, one that cleaves upon reaction with water (i.e., is hydrolyzed), e.g., under physiological conditions, such as for example, hydrolysis of an ester bond or of a succinimide ring thereby resulting in ring opening. The tendency of a bond to hydrolyze in water may depend not only on the general type of linkage connecting two atoms but also on the substituents attached to these atoms. Exemplary hydrolytically unstable or weak linkages may include but are not limited to carboxylate ester linkages, phosphate ester linkages, anhydride linkages, acetal linkages, ketal linkages, acyloxyalkyl ether linkages, imine linkages, orthoester linkages, peptide linkages, oligonucleotide linkages, thioester linkages, and carbonate linkages. Releasable linkages also include enzymatically releasable linkages, where an “enzymatically releasable linkage” means a linkage that is subject to cleavage by one or more enzymes. Additional types of release mechanisms include but are not limited to 1,6-benzyl elimination, β-elimination, and the like. While certain bonds may be considered to be stable or releasable, such characterization should be considered within the overall structure of a molecule or structural entity. In certain instances, a polymer conjugate containing a releasable bond may be referred to as a prodrug, wherein upon cleavage of a releasable bond in vivo (i.e., under physiological conditions), the parent drug is released (or may be eventually released, depending upon the number of polymeric moieties releasably attached to an active agent). A covalent “releasable” linkage, for example, in the context of a water-soluble polymer such as polyethylene glycol that is covalently attached to an active moiety such as IFN-γ, is one that cleaves under physiological conditions to thereby release or detach a water-soluble polymer from the active moiety, or to detach an active moiety from a water-soluble polymer.

A “stable” linkage or bond refers to a chemical bond that is substantially stable in water (e.g., under physiological conditions), that is to say, does not undergo hydrolysis under physiological conditions to any appreciable extent over an extended period of time. Examples of hydrolytically stable linkages generally include but are not limited to the following: carbon-carbon bonds (e.g., in aliphatic chains), ether linkages, amide linkages, carbamate linkages, amine linkages, and the like as well as combinations thereof. Generally, a stable linkage is one that exhibits a rate of hydrolysis of less than about 1-2% per day under physiological conditions. Hydrolysis rates of representative chemical bonds can be found in most standard chemistry textbooks. Further, one of ordinary skill in the art can determine whether a given linkage is stable or releasable in a given context by, for example, placing a linkage-containing molecule of interest under conditions of interest (e.g., under physiological conditions) and testing for evidence of release over a suitable time period.

A “PD-1 inhibitor” or “PD-1 antagonist” is any compound (such as a small molecule, ligand, or antibody) which inhibits binding of a programmed cell death protein 1 receptor (PD-1 receptor) with any of its ligands (e.g., PD-L1 and PD-L2). A “PD-L1 inhibitor” or “PD-L1 antagonist” is any compound (such as a small molecule, ligand, or antibody) which inhibits binding of a PD-1 receptor with the PD-L1 ligand. As used herein, “PD-1/PD-L1 axis inhibitor” refers to PD-1 inhibitors/antagonists as well as PD-L1 inhibitors/antagonists generally as well as specifically unless apparent otherwise by context.

As used herein in reference to treatment of a subject having cancer, the terms “treatment,” “treat,” and “treating” are meant to include the full spectrum of intervention for the cancer from which the subject is suffering, such as administration of the combination to alleviate, slow, stop, or reverse one or more symptoms of the cancer or to delay the progression of the cancer even if the cancer is not actually eliminated. Treatment can include, for example, a decrease in the severity of a symptom, the number of symptoms, or frequency of relapse, e.g., the inhibition of tumor growth, the arrest of tumor growth, or the regression of already existing tumors. Further, the term “treating cancer” is not intended to be an absolute term, and may include, for example, reducing the size of a tumor or number of cancer cells, causing a cancer to go into remission, or preventing growth in size or number of cancer cells, and the like. In some circumstances, treatment in accordance with the instant disclosure leads to an improved prognosis. For example, an improvement in the cancer or a cancer-related disease may be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

As used herein, the term “enhanced” or “enhancing”, for example, in the context of an enhanced response, refers to a subject's or tumor cell's improved ability to respond to treatment, e.g., as disclosed herein, when compared to a given baseline or reference therapy. For example, an enhanced response may comprise an increase in responsiveness of at least 5%, 10%, 15%, 20%, 25%, 30%, 35%, 40%, 45%, 50%, 55%, 60%, 65%, 70%, 75%, 80%, 85%, 90%, 95% or 98% or more, based upon any one or more indicators of responsiveness to treatment. As used herein, “enhanced” or “enhancing” can also refer to enhancing the number of subjects who favorably respond to treatment, e.g., when compared to a given basis for such comparison.

The terms “cancer” and “cancerous” refer to or describe the physiological condition in mammals that is typically characterized by unregulated cell growth.

“Tumor” and “solid tumor” as used herein refer to all lesions and neoplastic cell growth and proliferation, whether malignant or benign, and all pre-cancerous and cancerous cells and tissues. “Liquid cancer” as used herein refers to cancers that affect the bone marrow, blood cells, and/or the lymphatic system.

The term “patient,” or “subject” as used herein refers to a living organism suffering from or prone to a condition that can be prevented or treated by administration of a compound, composition, or combination as provided herein. Subjects or patients include, but are not limited to, mammals (e.g., murines, simians, equines, bovines, porcines, canines, felines, and the like), and preferably are human.

“Pharmacologically effective amount,” “physiologically effective amount,” and “therapeutically effective amount” are used interchangeably herein to mean the amount of an active agent, such as, for example, an IFN-γ mutein polymer conjugate, that is needed to provide a desired level of active agent and/or conjugate in the bloodstream or in the target tissue. The precise amount may depend upon numerous factors, e.g., the particular active agent, the components and physical characteristics of the composition, intended patient population, patient considerations, and may readily be determined by one skilled in the art, based upon the information provided herein and available in the relevant literature. For example, a therapeutically effective amount of a compound, or a combination of one or more compounds, when administered (either sequentially or concomitantly) is an amount that elicits a desired biological or medicinal response, e.g., either destroys cancer cells, slows or arrests the progression of a cancer in a subject, and/or increases the surface expression of the major histocompatibility complex class I (MHCI) and/or major histocompatibility complex class II (MHCII) molecules and the processing and presenting of antigens. The term also applies to a dose of the compounds that will induce a particular desired response in target cells, e.g., when administered in combination, to provide in a beneficial effect. In certain embodiments, the combined effect is additive, while in certain other embodiments, the combined effect is synergistic. Further, it will be recognized by one skilled in the art that in the case of combination therapy, the amount of each of the separate therapeutic agents, e.g. IFN-γ and/or a checkpoint inhibitor such as a PD-1/PD-L1 axis inhibitor, may be used in a “sub-therapeutic amount”, i.e., less than the therapeutically effective amount of such compound when administered alone.

Combination therapy or “in combination with” refers to the use of more than one therapeutic agent to treat a particular disorder or condition. By “in combination with,” it is not intended to imply that the therapeutic agents must be administered at the same time and/or formulated for delivery together, although these methods of delivery are within the scope of this disclosure. A therapeutic agent can be administered concurrently with, prior to, or subsequent to, one or more other additional agents on the same or different days. The therapeutic agents in a combination therapy can also be administered on an alternating dosing schedule, with or without a resting period (e.g., no therapeutic agent is administered on certain days of the schedule). The administration of a therapeutic agent “in combination with” another therapeutic agent includes, but is not limited to, sequential administration and concomitant administration of the two or more agents. In general, each therapeutic agent is administered at a dose and/or on a time schedule determined for that particular agent.

“Pharmaceutically acceptable excipient” or “pharmaceutically acceptable carrier” refers to a component, other than the pharmacologically active agent, that may be included in the compositions described herein and causes no significant adverse toxicological effects to a subject.

“Substantially” or “essentially” means nearly totally or completely, for instance, 95% or greater of a given quantity.

Similarly, “about” or “approximately” as used herein means within plus or minus 5% of a given quantity.

“Optional” or “optionally” means that the subsequently described circumstance may, but need not necessarily, occur so that the description includes instances where the circumstance occurs and instances where it does not. “Optional” or “optionally” also means that variables or components described may, but need not necessarily, be present so that the description includes instances where the variables or components are present and instances where they are not present.

Amino acid residues in peptides are abbreviated as follows: Phenylalanine is Phe or F; Leucine is Leu or L; Isoleucine is IIe or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

An exemplary conjugate, active moiety, or other suitably applicable chemical moiety as described herein is meant to encompass, where applicable, analogues, isomers, polymorphs, solvates, and pharmaceutically acceptable salt forms thereof.

In the context of the present disclosure, it should be recognized that the definition of a variable provided with respect to one structure or formula is applicable to the same variable repeated in a different structure, unless the context dictates otherwise.

### Overview

The water-soluble polymer IFN-γ mutein conjugates described herein incorporate a number of innovative advances in drug design and treatment rationale that integrate into a novel, potentially safer and highly efficacious therapy such as an anti-cancer therapy. In particular, the polymer modified IFN-γ muteins described herein, e.g. poly(ethylene glycol) conjugated muteins of IFN-γ, were optimized to possess reduced receptor binding affinity and/or reduced signaling potency as compared to unmodified IFN-γ and/or other polymer modified IFN-γ muteins. Further, certain polymer modified IFN-γ muteins as described herein, e.g. poly(ethylene glycol) conjugated IFN-γ muteins, were discovered to exhibit reduced heparin binding affinity as compared to IFN-γ or other modified IFN-γ muteins. Additionally, the polymer modified IFN-γ muteins described herein provide one or more of (i) sustained induction of major histocompatibility complex (MHC) molecules, and (ii) neoantigen presentation by tumor cells to trigger T cell recognition leading to increased tumor infiltration. The polymer modified IFN-γ muteins described herein preferably bias toward IFN-γ anti-tumor activity including, but not limited to upregulation of and maturing of MHC class I molecules (MHCI) and/or MHC class II molecules (MHCII) on dendritic cells and the resulting increase in antigen presentation, stimulation of macrophages toward the proinflammatory M1 phenotype, and stimulation of Th1 cell differentiation of T cells.

As noted above, IFN-γ is a potent, short-lived cytokine that exhibits toxicity at high doses. In some embodiments, the present disclosure is based at least in part on the use of site-specific PEGylation at an engineered cysteine residue within or near the interferon-γ receptor-1 (IFNGR1) binding site in order to reduce the binding affinity and signaling potency of IFN-γ while extending exposure and induction of MHCI and/or MHCII. The reduced-potency conjugate is expected to have therapeutically desirable benefits over the native cytokine, including but not limited to: improved pharmacokinetic properties due to reduced target-mediated clearance; decreased induction of known negative feedback and counterregulatory mechanisms, decreased receptor internalization and associated tachyphylaxis; and/or an expanded therapeutic index.

The present disclosure is further based, at least in part, on the use of strategic PEGylation of hIFN-γ muteins in order to mitigate the binding of heparin to hIFN-γ to thereby mitigate the inhibition of hIFN-γ effects by heparin. Unlike a number of other cytokines, IFN-γ has a direct effect in limiting tumor cell proliferation by direct anti-proliferative or pro-apoptotic mechanisms. Heparin has been shown to be an antagonist of IFN-γ and binding of heparin to IFN-γ has been shown to inhibit IFN-γ signaling in human endometrial stromal cells (Fluhr (2011)). As shown herein in Examples 23 and 24, certain poly(ethylene glycol) conjugated muteins of IFN-γ-1 b are more resistant to heparin binding and the resulting inhibition of IFN-γ activity as compared to IFN-γ or other modified IFN-γ muteins.

Molecules designed to generate these potential benefits, including reduced heparin affinity and/or extended exposure, may possess enhanced anti-tumor or other biological properties compared to unmodified IFN-γ through multiple mechanisms.

### IFN-γ Conjugates

Cysteine residues do not occur abundantly in proteins generally, and account for less than one percent of the total amino acid content of proteins. Moreover, cysteines that do occur in proteins often form disulfide bonds, thus making them unavailable for reaction with many thiol-specific PEGylation reagents. Wild type or native human IFN-γ does not contain any cysteine residues. IFN-γ as described herein is a cysteine mutein where the IFN-γ protein or polypeptide is modified to include one or more cysteine amino acid residues at specific locations in the IFN-γ protein. Such modification includes insertion of one or more cysteine residues by introduction of one or more cysteines between two amino acids of the wild type IFN-γ at one or more positions and/or substitution of one or more amino acids with a cysteine, in order to provide facile attachment of a water-soluble, non-peptidic polymer to an atom within the side chain of the cysteine. That is, one or more cysteine amino acids may be added/inserted and/or one or more cysteine amino acids may be substituted to prepare the IFN-γ muteins for use as described herein. Techniques for adding amino acid residues and non-naturally occurring amino acid residues are well known to those of ordinary skill in the art (e.g. Bioinformatics for Geneticists (eds. Michael R. Barnes and Ian C. Gray), 2003 John Wiley & Sons, Ltd, Chapter 14, Amino Acid Properties and Consequences of Substitutions, Betts, M. J., and Russell, R. B). See also, for example, the procedure described in PCT Publication No. WO 90/12874 (Genetics Institute, Inc.), incorporated herein by reference, for adding cysteine residues, wherein such procedure can be adapted for IFN-γ. An exemplary procedure for the design and presentation of cysteine substituted IFN-γ muteins is provided in Example 1.

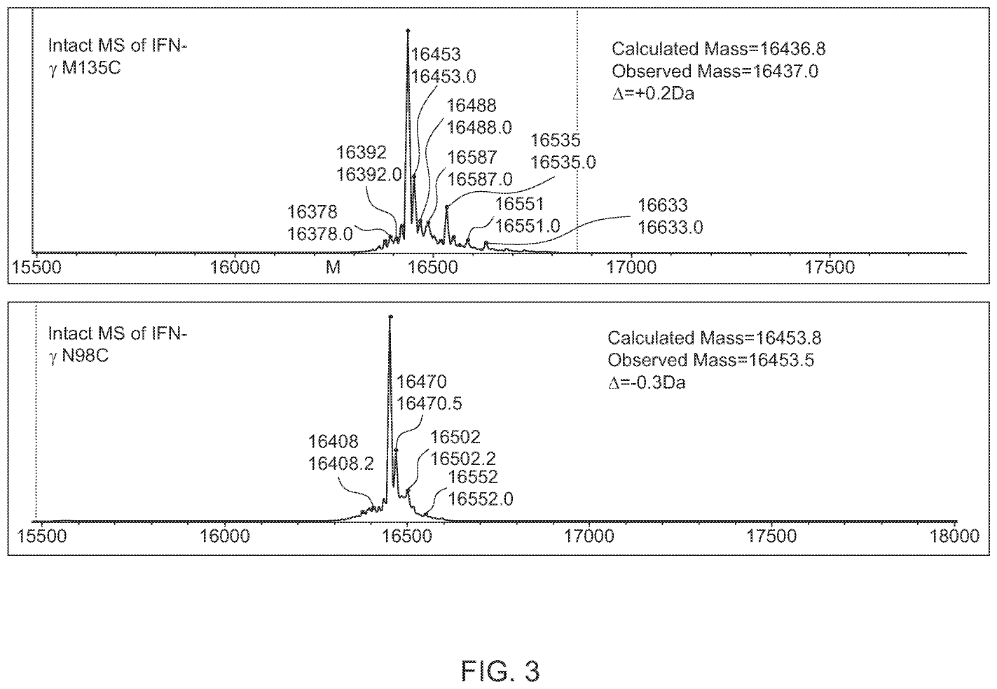

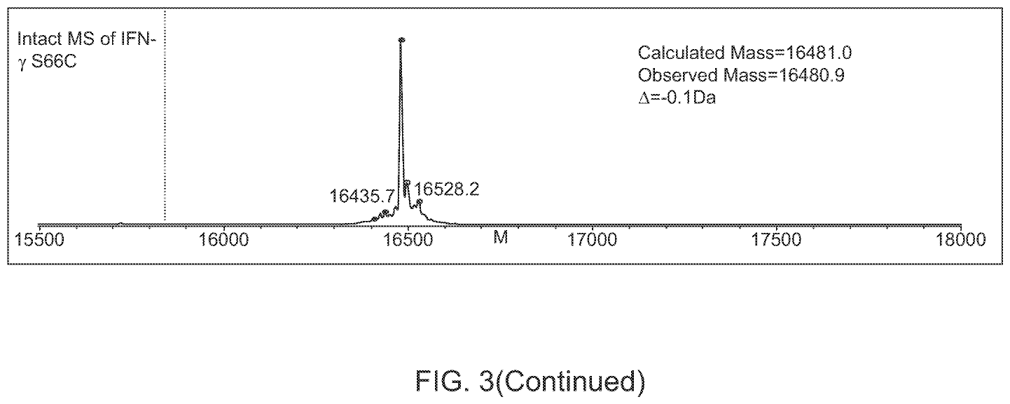

In one embodiment, one or more cysteine residues, and preferably a single cysteine residue, is introduced by insertion or substitution within or near the IFNGR1 binding site of the IFN-γ protein. The structure of the IFN-γ protein dimer in complex with IFNGR1 has been determined (Mendoza et al., Nature, 567:56-60 (2019), FIG. 3). The IFNGR1 binding site of an IFN-γ protein can readily be determined with reference to the IFN-γ protein:IFNTR1 complex by identifying amino acids involved in binding (that is, the IFNGR1 binding site of the IFN-γ protein). Thereafter, an IFN-γ cysteine mutein may be designed having at least one cysteine insertion or substitution within or near the determined IFNGR1 binding site. In some embodiments, the IFN-γ cysteine mutein comprises at least one cysteine insertion or substitution within the C-terminus of IFN-γ. As used herein, the C-terminus refers to the C-terminal 20-25 amino acids of the human IFN-γ protein.

In another embodiment, one or more cysteine residues, and preferably a single cysteine residue, is introduced by insertion or substitution within or near the heparin binding domain in the IFN-γ carboxyl-terminal domain. The amino acid sequence of the heparin binding domain of IFN-γ-1b is KTGKRKRSQMLFRGR (SEQ ID NO:8), amino acids 125-140 of IFN-γ-1b.

As used herein “near” with reference to the position of the introduced cysteine relative to the IFNGR1 binding domain or heparin binding domain means that introduction of the cysteine is such that the attached polymer at least partially inhibits binding to the IFNGR1 or heparin, respectively, or both. In some embodiments, the cysteine insertion or substitution is located within 1-10 amino acids from either end of the binding domain. In some exemplary embodiments, the cysteine insertion or substitution is located within about 1-5 amino acids, or about 5-10 amino acids from either end of the binding domain, or the cysteine insertion or substitution is located within at least one amino acid from either end of the binding domain. It will be appreciated that the cysteine may be introduced within the IFNGR1 binding domain or the heparin binding domain, or both. Exemplary substitutions include, but are not limited to, serine at position 66 to cysteine (S66C), asparagine at position 98 to cysteine (N98C), and methionine at position 135 to cysteine (M135C) of SEQ ID NO:3 or SEQ ID NO:7. Example 1 shows preparation of IFN-γ muteins having a substitution at each of S66C, N98C, and M135C. In one or more embodiments, IFN-γ muteins can be prepared according to a method as described in Example 1. Further suitable substitutions may be identified by reference to the structure of the IFN-γ protein dimer in complex with IFNGR1, which has been determined (see Mendoza et al., Nature, 567:56-60 (2019)) in that amino acids on the IFN-γ protein that are involved in binding to the IFNGR1 are suitable for cysteine substitution.

The IFN-γ mutein can be expressed in bacterial (e.g., E. coli, see, for example, Fischer et al. (1995) Biotechnol. Appl. Biochem., 21(3):295-311), mammalian (see, for example, Kronman et al. (1992) Gene, 121:295-304), yeast (e.g., Pichia pastoris, see, for example, Morel et al. (1997) Biochem. J., 328(1):121-129), and plant (see, for example, Mor et al. (2001) Biotechnol. Bioeng., 75(3):259-266) expression systems. The expression can occur via exogenous expression (when the host cell naturally contains the desired genetic coding) or via endogenous expression.

Although recombinant-based methods for preparing proteins can differ, recombinant methods typically involve constructing the nucleic acid encoding the desired polypeptide or fragment, cloning the nucleic acid into an expression vector, transforming a host cell (e.g., plant, bacteria, yeast, transgenic animal cell, or mammalian cell such as Chinese hamster ovary (CHO) cell or baby hamster kidney cell), and expressing the nucleic acid to produce the desired polypeptide or fragment. Methods for producing and expressing recombinant polypeptides in vitro and in prokaryotic and eukaryotic host cells may be used as known to those of ordinary skill in the art.

Depending on the system used to express IFN-γ, the protein can be unglycosylated or glycosylated and either may be used. In one or more embodiments, the IFN-γ mutein is unglycosylated.

For any given IFN-γ mutein or conjugate, it is possible to determine whether that mutein or conjugate has IFN-γ activity. Various methods for determining in vitro IFN-γ activity are described in the art. An exemplary approach is based on a STAT1 phosphorylation assay as described in Example 17.

To prepare IFN-γ mutein conjugates, reaction of the cysteine mutein with a thiol-selective or thiol-specific water-soluble, non-peptidic polymer reagent, e.g. a thiol-selective or thiol-specific PEGylation reagent, is carried out to provide an IFN-γ protein with a PEG moiety covalently attached at the particular cysteine insertion site(s). PEG reagents suitable for reaction with cysteines include those with reactive groups such as thiol, disulfide, maleimide, vinyl sulfone, halide, orthopyridyl disulfide (OPSS), and iodoacetamide, and such reagents are suitable for forming an IFN-γ cysteine mutein conjugate as described herein. Activated PEG reagents suitable for reaction with a thiol group are commercially available from vendors such as NOF Corporation, Creative PEGworks, Biopharma PEG, Sigma Aldrich, and the like. One particularly preferred approach for preparing an IFN-γ cysteine mutein conjugate via cysteine-directed site-specific PEGylation involves reaction of an IFN-γ cysteine-mutein with a maleimide-functionalized PEG reagent. Exemplary polymer reagents, IFN-γ cysteine muteins, and reaction conditions for preparing the subject conjugates are described in Examples 4-11, and such are suitable or may be readily adapted in light of the teachings provided herein, when considered along with knowledge commonly available in the chemical and polymer arts, for forming IFN-γ cysteine-mutein conjugates having one or more of the advantageous features described herein. A preferred IFN-γ conjugate comprises a single, linear or branched water-soluble, non-peptidic polymer (e.g. polyethylene glycol or PEG) covalently attached to a cysteine amino acid in the IFN-γ mutein via a thioether linkage of the IFN-γ monomer (e.g. the IFN-γ monomer is mono-PEGylated). The IFN-γ conjugates for use herein are preferably but not necessarily in the form of a homodimer where each monomer of the dimer is a cysteine mutein as described herein, and where each monomer comprises a single linear or branched water-soluble, non-peptidic polymer (e.g. polyethylene glycol or PEG) covalently attached to a cysteine amino acid in the IFN-γ mutein via a thioether linkage. It will be appreciated, however that in some instances, only one monomer of the homodimer may be PEGylated. It will further be appreciated that the IFN-γ conjugates may also be in the form of a heterodimer where each monomer of the dimer is a cysteine mutein as described herein and comprises a single, linear or branched water-soluble, non-peptidic polymer (e.g. polyethylene glycol or PEG) covalently attached to a cysteine amino acid in the IFN-γ mutein via a thioether linkage where each monomer utilizes a different PEG and/or linkage. Conjugate compositions as described herein may comprise all or substantially all IFN-γ homodimers, all or substantially all IFN-γ heterodimers, or a mixture of homodimers and heterodimers. Preferred compositions are those comprising at least about 75 mole percent of IFN-γ conjugates that are homodimers, where in some embodiments, both “monomer” portions of the IFN-γ mutein conjugate comprise a formula or structure as set forth herein, including all relevant embodiments, where both succinimidyl groups (resulting from reaction of a maleimide-functionalized PEG reagent with a thiol group of the IFN-γ cysteine mutein) are in a ring-open form.

In preferred embodiments, the water-soluble, non-peptidic polymer is a poly(alkylene oxide) such as a poly(alkylene glycol). A preferred poly(alkylene glycol) is poly(ethylene glycol). The polymer is not limited to a particular structure and can be linear or branched. When the water-soluble, non-peptidic polymer is a poly(ethylene glycol), the polymer will comprise a number of (OCH₂CH₂) monomers or (CH₂CH₂O) monomers, depending on how the poly(ethylene glycol) is defined. As used throughout the description, the number of repeat units is identified by the subscript “n” in “(OCH₂CH₂)ₙ.” In one or more embodiments, n is an integer having a value that corresponds to a polyethylene glycol polymer having a weight average molecular weight selected from the group consisting of about 2,000 daltons (where n is ˜45), about 5,000 daltons (where n is ˜113), or about 10,000 daltons (where n is ˜227), or about 15,000 daltons (where n is ˜340), or about 20,000 daltons (where n is ˜454), or about 25,000 daltons (where n is ˜568), or about 30,000 daltons (where n is ˜681), or about 40,000 daltons (where n is ˜909), or about 50,000 daltons (where n is ˜1136) or about 60,000 daltons (where n is ˜1364), or about 80,000 daltons (where n is ˜1818), or greater. For any given polymer in which the molecular weight is known, it is possible to determine the number of repeating units (i.e., “n”) by dividing the total weight-average molecular weight of the polymer by the molecular weight of the repeating monomer unit.

Further exemplary weight average molecular weights for the polyethylene glycol portion of the conjugate, that is for each IFN-γ monomer, in addition to the foregoing, include about 11,000 daltons, about 12,000 daltons, about 13,000 daltons, about 14,000 daltons, about 22,500 daltons, about 35,000 daltons, about 45,000 daltons, about 55,000 daltons, about 65,000 daltons, about 70,000 daltons, and about 75,000 daltons. In some embodiments, the weight-average molecular weight of the polyethylene glycol portion of each IFN-γ monomer of the conjugate is about 10,000 to about 80,000 daltons.

Where the IFN-γ mutein is attached to a branched PEG polymer, it will be appreciated that each arm of the branched polymer may have the same or different weight-average molecular weight. Thus, the weight average molecular weight of the of each arm may be the same for each arm or may be different between the arms.

The thiol group(s) of the IFN-γ cysteine mutein can serve as effective sites of attachment for the water-soluble polymer. The thiol groups in such cysteine residues can be reacted with an activated water-soluble polymer reagent such as an activated poly(ethylene glycol) reagent that is specific for reaction with thiol groups, e.g., an N-maleimidyl polymer reagent or other derivative as described in U.S. Pat. No. 5,739,208 and in PCT Publication No. WO 01/62827, both of which are incorporated herein by reference. Suitable PEG reagents may be synthesized or may be obtained from commercially available sources.

Specific examples of suitable reagents, along with the corresponding conjugate, are provided in Table 1, below. In the table, the variable (n) represents the number of repeating monomeric units and “—S-(IFN-γ mutein)” represents the IFN-γ mutein following conjugation to the water-soluble polymer. While each polymeric portion [e.g., (OCH₂CH₂)ₙ or (CH₂CH₂O)ₙ]presented in Table 1 terminates in a “CH₃” group, other end-capping groups as described herein can be substituted therefor. The following list of exemplary reagents and resulting conjugates is in no way intended to be limiting but rather to be illustrative.

TABLE 1
Thiol-Selective Polymeric Reagents and the IFN-y Cysteine Mutein Conjugate
Formed Therefrom
Polymeric Reagent

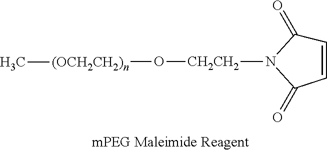

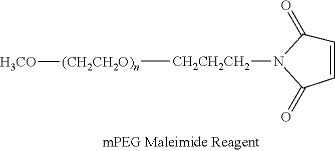

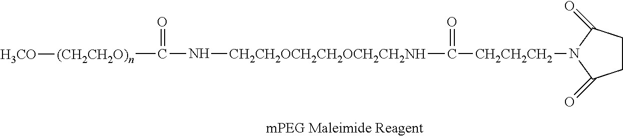

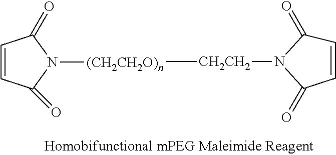

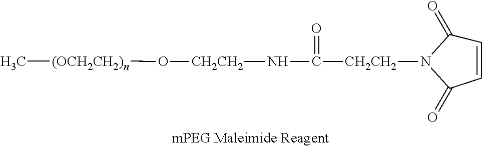

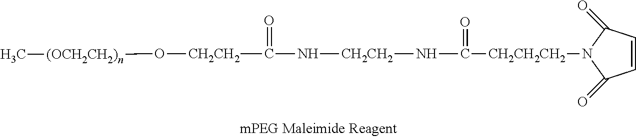

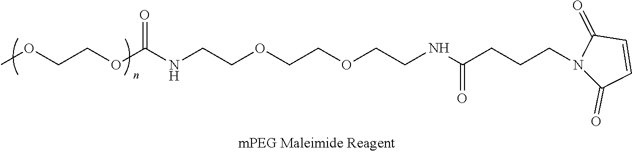

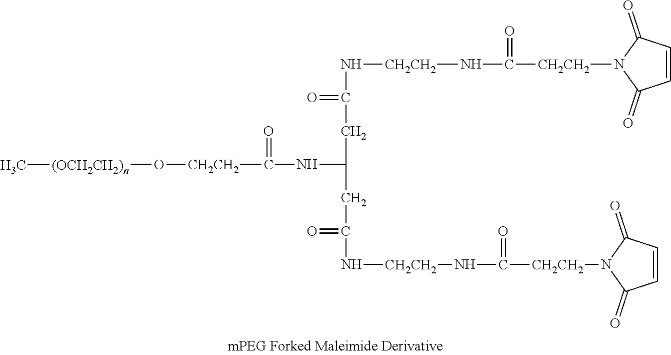

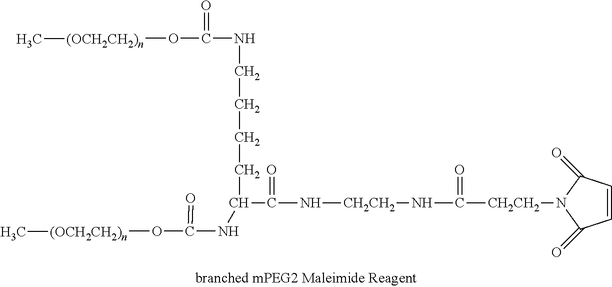

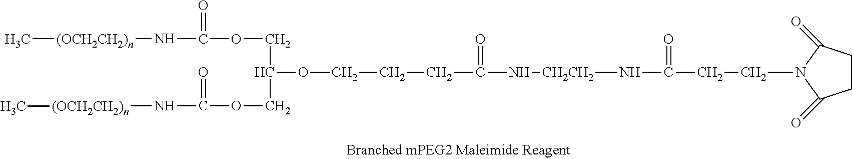

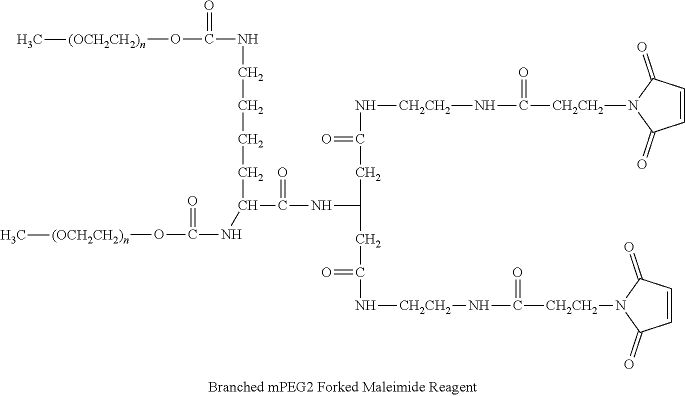

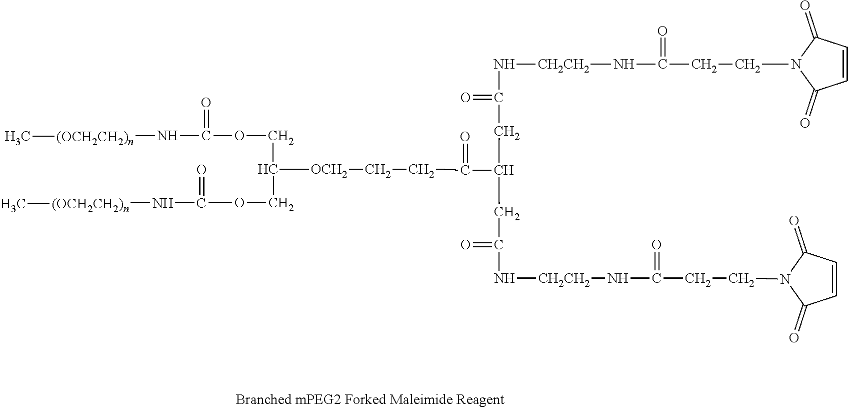

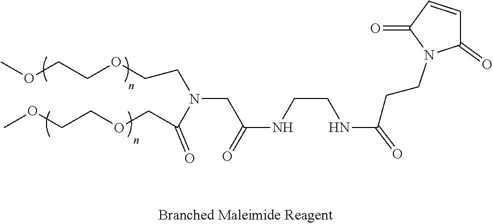

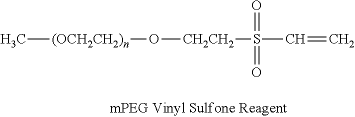

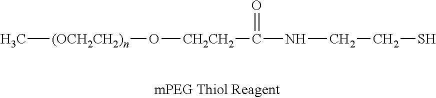

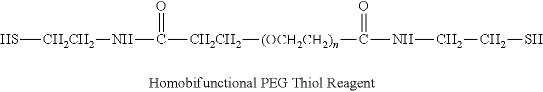

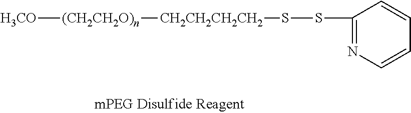

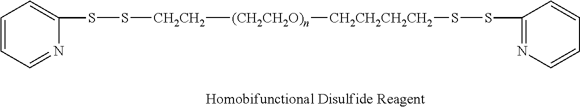

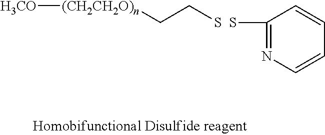

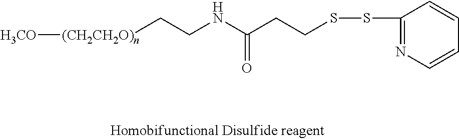

Corresponding Conjugate

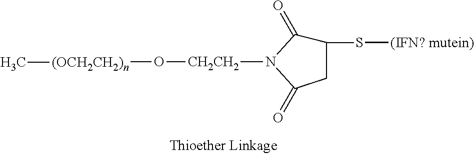

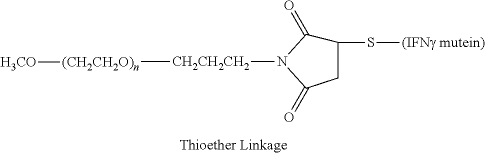

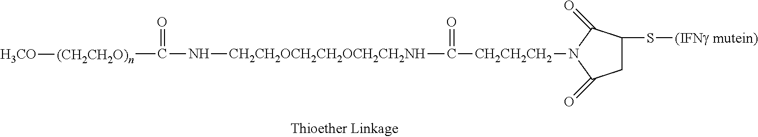

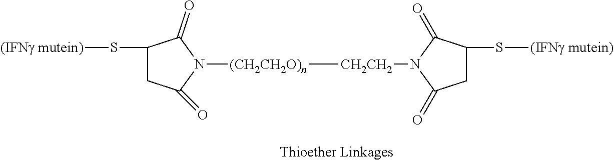

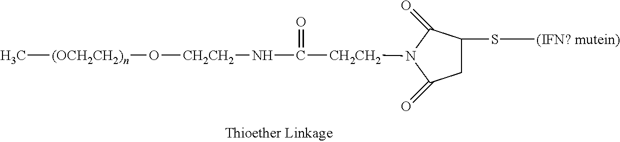

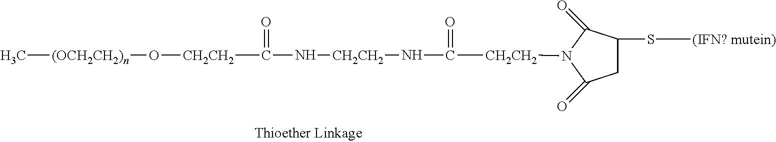

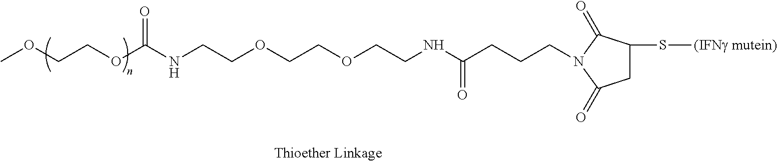

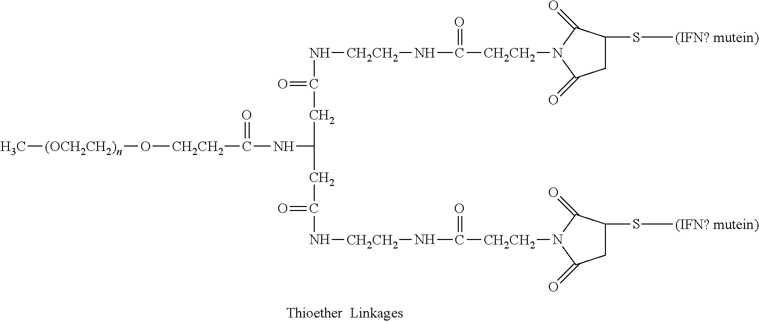

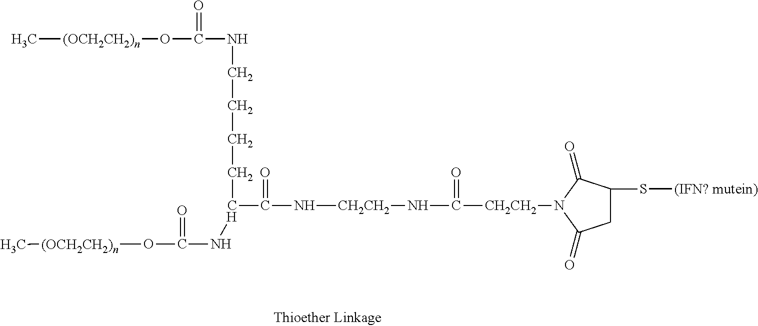

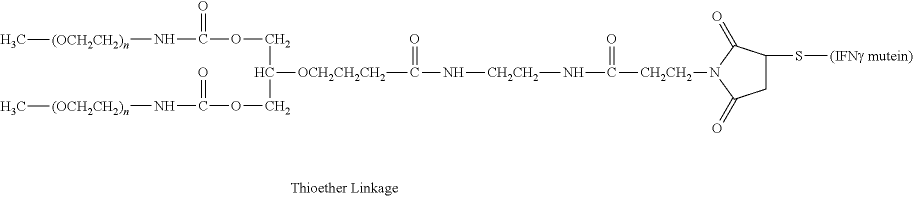

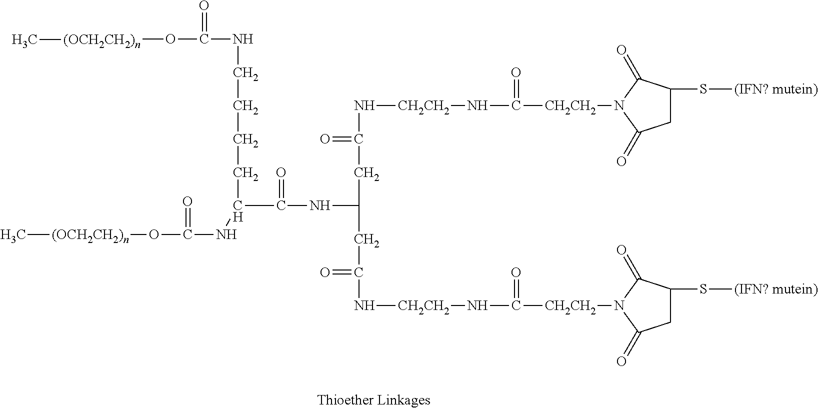

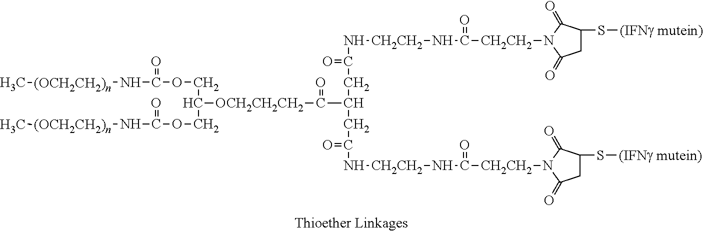

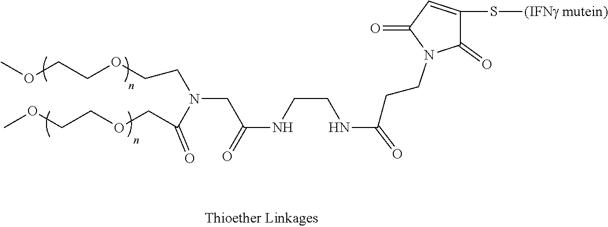

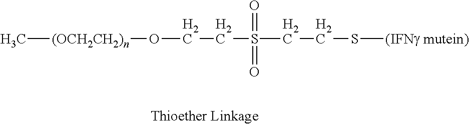

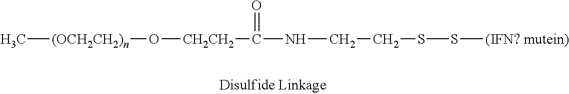

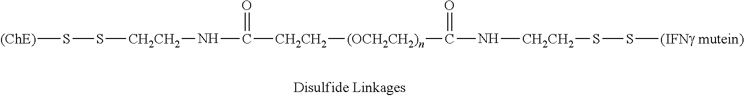

H₃CO—(CH₂CH₂O)ₙ—CH₂CH₂CH₂CH₂—S—S—(IFNγ mutein) Disulfide Linkage
(IFNγ mutein)—S—S—CH₂CH₂—(CH₂CH₂O)ₙ—CH₂CH₂CH₂CH₂—S—S—(IFNγ mutein) Disulfide Linkage

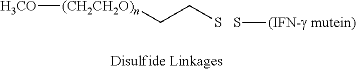

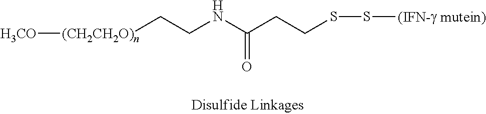

Conjugates can be formed using thiol-selective polymeric reagents in a number of ways and the invention is not limited in this regard. For example, the IFN-γ cysteine mutein, optionally in a suitable buffer, is placed in an aqueous medium at a pH of about 7-8 and the thiol-selective polymeric reagent is typically added at a molar excess. The activated PEG reagent may be added at a molar excess ranging from about two-fold to about one hundred-fold (e.g., 5-fold excess, 10-fold excess, 20-fold excess, 30-fold excess, 40-fold excess, 50-fold excess, 60-fold excess, etc.). The reaction may be carried out at room temperature, between about 15 and 30 degrees Celsius, however, in some instances, the reaction may be carried out at temperatures ranging from about 4 degrees Celsius to about 60 degrees Celsius, and a suitable reaction temperature can be readily determined. The reaction is allowed to proceed for about 0.5 to 2 hours, although reaction times of greater than 2 hours (e.g., 5 hours, 10 hours, 12 hours, and 24 hours or even longer) may be used if PEGylation yields are determined to be relatively low. Appropriate reaction conditions can be determined by adjusting various reaction parameters, and such is within the skill in the art. Exemplary polymeric reagents that can be used in this approach are polymeric reagents bearing a reactive group selected from the group consisting of maleimide, sulfone (e.g., vinyl sulfone), and thiol (e.g., functionalized thiols such as an ortho pyridinyl or “OPSS”). Examples 4-10 provide details of suitable reaction methods of forming exemplary Compounds 1-9.

In some embodiments, the IFN-γ conjugate has the structure of Formula I:

IFN-γ-s-x-POLY Formula I

where IFN-γ is a cysteine modified mutein, —S— is a sulfur atom of the inserted or substituted cysteine, X is a spacer moiety as described further herein, and POLY is a water-soluble, non-peptidic polymer as also described further herein. In Formula I (and in similar formulae described herein) the —S— in the structure, prior to conjugation, is a thiol group of a cysteine that has been substituted or inserted into the IFN-γ molecule.

The attachment between the IFN-γ cysteine mutein and the water-soluble, non-peptidic polymer can be direct, wherein no intervening atoms are located between the IFN-γ cysteine mutein and the polymer (with the exception of the linking chemical group or atom, such as in Formula I, the thioether, or indirect, wherein one or more atoms are located between the IFN-γ cysteine mutein and the polymer. With respect to the indirect attachment, a “spacer moiety”, “spacer”, “linker”, “linkage”, or “spacer linkage” serves as a spacer or linker between the sulfur residue of the IFN-γ cysteine mutein and the water-soluble polymer. The spacer or linker typically has a chain length of from about 1 to about 25 atoms, from about 3 to about 20 atoms, from about 1 to about 10 atoms, or from about 1-5 atoms, or from about 5 to about 18 atoms, whereby length is meant the number of atoms in a single chain, not counting substituents. For instance, a carbamate linkage (R—NH—(C═O)—NH—R′), is considered to have a chain length of 3 atoms. The one or more atoms making up the linker or spacer moiety can include one or more of carbon atoms, nitrogen atoms, sulfur atoms, oxygen atoms, and combinations thereof. The spacer moiety can comprise a carbon-carbon bond, an amide, secondary amine, carbamate, thioether, and/or disulfide group. In considering chain “length” in a ring structure, such as a succinimide, the number of atoms is the smallest number of atoms in progressing directionally around the ring (clockwise or counterclockwise). For example, in Compound 3, the contribution to spacer length by the succinimide ring is 3 atoms, starting with the nitrogen atom and progressing counterclockwise to the carbon to which the sulfur atom is attached.

Nonlimiting examples of specific spacer moieties include those selected from the group consisting of —O—, —S—, —S—S—, —C(O)—, —C(O)—NH—, —NH—C(O)—NH—, —O—C(O)—NH—, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂—C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—NH—C(O)—CH₂—CH₂—, —O—C(O)—NH—[C H₂]ₕ—(OCH₂CH₂)ⱼ—, bivalent cycloalkyl group, —O—, —S—, an amino acid, —N(R⁶)—, and combinations of two or more of any of the foregoing, wherein R⁶ is H or an organic radical selected from the group consisting of alkyl, substituted alkyl, alkenyl, substituted alkenyl, alkynyl, substituted alkynyl, aryl and substituted aryl, (h) is zero to six, and (j) is zero to 20. The above collection of atoms are to be considered both from left to right and from right to left. Other specific spacer moieties have the following structures: —C(O)—NH—(CH₂)₁₋₆—NH—C(O)—, —NH—C(O)—NH—(CH₂)₁₋₆—NH—C(O)—, and —O—C(O)—NH—(CH₂)₁-6-NH—C(O)—, wherein the subscript values following each methylene indicate the number of methylenes contained in the structure, e.g., (CH₂)₁₋₆ means that the structure can contain 1, 2, 3, 4, 5 or 6 methylenes. In some embodiments, the spacer moiety comprises the structure:

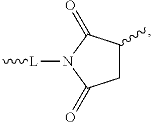

where L is a linker or spacer as described above comprising one or more atoms selected from carbon, nitrogen, sulfur, oxygen, and combinations thereof. In some preferred embodiments, the spacer moiety comprises the structure:

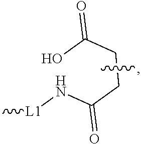

wherein L1 is a linker comprising one or more atoms selected from carbon, nitrogen, sulfur, oxygen, combinations thereof, and ad described further above. In other embodiments, the spacer or linker is ˜(CH₂)ₐ(O)_{b}[C(O)]_{c}(NH)_{d}(CH₂)ₑ(NH)_{f}[C(O)]_{g}(CH₂)ₕ˜, wherein: a is 0-10; b is 0, 1; c is 0, 1; d is 0, 1; e is 0-10; f is 0, 1; g is 0, 1; and h is 0-10, wherein at least one of a, b, c, d, e, f, g, and h is a positive integer. For example, variable “a” may have a value of 0, 1, 2, 3, 4, 5, 6, 7, 8, 9, or 10. That is, the —CH₂—group may be absent (a=0), or may be one of the following: —(CH₂), —(CH₂)₂, —(CH₂)₃, —(CH₂)₄, —(CH₂)₅, —(CH₂)₆, —(CH₂)₇, —(CH₂)₈, —(CH₂)₉, or —(CH₂)₁₀. In some embodiments, b is zero (such that the oxygen atom is absent. In some other embodiments, b is 1 (such that the oxygen atom is present). Similarly, in some embodiments, c is zero (i.e., is absent). In some other embodiments, c is 1 (i.e., c is present). Similarly, in some embodiments, d is zero (i.e., is absent). In some other embodiments, d is 1 (i.e., d is present). In some embodiments, the linker comprises an amide function such that both c and d are one (or both f and g are one). In some embodiments, variable “e” has a value of 0, 1, 2, 3, 4, 5, 6, 7, 8, 9, or 10. That is, the —CH₂—group may be absent (e=0), or may be one of the following: —(CH₂), —(CH₂)₂, —(CH₂)₃, —(CH₂)₄, —(CH₂)₅, —(CH₂)₆, —(CH₂)₇, —(CH₂)₈, —(CH₂)₉, or —(CH₂)₁₀. In some embodiments, f is 0 (the —NH—group is absent). In some embodiments, f is 1 (the —NH— group is present). In some embodiments, g is 0 (such that the carbonyl carbon is absent); in some embodiments, g is 1 (such that the carbonyl carbon is present). In some embodiments, h is zero (such that the —CH₂—group is absent). In some embodiments, h is a positive integer selected from 1-10, such that the corresponding collection of atoms is —(CH₂), —(CH₂)₂, —(CH₂)₃, —(CH₂)₄, —(CH₂)₅, —(CH₂)₆, —(CH₂)₇, —(CH₂)₈, —(CH₂)₉, or —(CH₂)₁₀. In some particular embodiments, f is 1, g is 1 and h is 2. In yet some further embodiments, a, b, c, d, and e are zero. In one or more further embodiments, a is 3, b is 0, c is 1, d is 1 and e is 2. In yet some additional embodiments, f is 1, g is 1, h is 2, and a, b, c, d, and e are zero. In yet some more particular embodiments of the linker, f is 1, g is 1, h is 2, a is 3, b is 0, c is 1, d is 1 and e is 2.

In one or more embodiments, POLY or the polyethylene glycol monomer portion thereof has a weight average molecular weight of about 1000 daltons to about 100,000 daltons. In some embodiments, POLY has a weight average molecular weight of about 2,000 to about 80,000 daltons, about 2,000 to about 60,000 daltons, about 2,000 to about 40,000 daltons, about 2,000 to about 20,000 daltons, about 2,000 to about 10,000 daltons, about 2,000 to about 5,000 daltons, about 10,000 to about 100,000 daltons, about 15,000 to about 100,000 daltons, about 20,000 to about 100,000 daltons, about 25,000 to about 100,000 daltons, about 30,000 to about 100,000 daltons, about 40,000 to about 100,000 daltons, about 45,000 to about 100,000 daltons, about 50,000 to about 100,000 daltons, about 55,000 to about 100,000 daltons, about 60,000 to about 100,000 daltons, about 65,000 to about 100,000 daltons, about 70,000 to about 100,000 daltons, about 75,000 to about 100,000 daltons, about 80,000 to about 100,000 daltons, about 90,000 to about 100,000 daltons, about 95,000 to about 100, daltons, about 10,000 to about 90,000 daltons, about 15,000 to about 90,000 daltons, about 20,000 to about 90,000 daltons, about 25,000 to about 90,000 daltons, about 30,000 to about 90,000 daltons, about 40,000 to about 90,000 daltons, about 45,000 to about 90,000 daltons, about 50,000 to about 90,000 daltons, about 55,000 to about 90,000 daltons, about 60,000 to about 90,000 daltons, about 65,000 to about 90,000 daltons, about 70,000 to about 90,000 daltons, about 75,000 to about 90,000 daltons, about 80,000 to about 90,000 daltons, about 10,000 to about 80,000 daltons, about 15,000 to about 80,000 daltons, about 20,000 to about 80,000 daltons, about 25,000 to about 80,000 daltons, about 30,000 to about 80,000 daltons, about 40,000 to about 80,000 daltons, about 45,000 to about 80,000 daltons, about 50,000 to about 80,000 daltons, about 55,000 to about 80,000 daltons, about 60,000 to about 80,000 daltons, about 65,000 to about 80,000 daltons, about 70,000 to about 80,000 daltons, about 75,000 to about 80,000 daltons, about 10,000 to about 75,000 daltons, about 15,000 to about 75,000 daltons, about 20,000 to about 75,000 daltons, about 25,000 to about 75,000 daltons, about 30,000 to about 75,000 daltons, about 40,000 to about 75,000 daltons, about 45,000 to about 75,000 daltons, about 50,000 to about 75,000 daltons, about 55,000 to about 75,000 daltons, about 60,000 to about 75,000 daltons, about 65,000 to about 75,000 daltons, about 70,000 to about 75,000 daltons, about 10,000 to about 70,000 daltons, about 15,000 to about 70,000 daltons, about 20,000 to about 70,000 daltons, about 25,000 to about 70,000 daltons, about 30,000 to about 70,000 daltons, about 40,000 to about 70,000 daltons, about 45,000 to about 70,000 daltons, about 50,000 to about 70,000 daltons, about 55,000 to about 70,000 daltons, about 60,000 to about 70,000 daltons, about 65,000 to about 70,000 daltons, about 10,000 to about 60,000 daltons, about 15,000 to about 60,000 daltons, about 20,000 to about 60,000 daltons, about 25,000 to about 60,000 daltons, about 30,000 to about 60,000 daltons, about 40,000 to about 60,000 daltons, about 45,000 to about 60,000 daltons, about 50,000 to about 60,000 daltons, about 55,000 to about 60,000 daltons, about 60,000 to about 60,000 daltons, about 10,000 to about 55,000 daltons, about 15,000 to about 55,000 daltons, about 20,000 to about 55,000 daltons, about 25,000 to about 55,000 daltons, about 30,000 to about 55,000 daltons, about 40,000 to about 55,000 daltons, about 45,000 to about 55,000 daltons, about 50,000 to about 55,000 daltons, about 10,000 to about 50,000 daltons, about 15,000 to about 50,000 daltons, about 20,000 to about 50,000 daltons, about 25,000 to about 50,000 daltons, about 30,000 to about 50,000 daltons, about 40,000 to about 50,000 daltons, about 45,000 to about 50,000 daltons, about 10,000 to about 45,000 daltons, about 15,000 to about 45,000 daltons, about 20,000 to about 45,000 daltons, about 25,000 to about 45,000 daltons, about 30,000 to about 45,000 daltons, about 40,000 to about 45,000 daltons, about 10,000 to about 40,000 daltons, about 15,000 to about 40,000 daltons, about 20,000 to about 40,000 daltons, about 25,000 to about 40,000 daltons, about 30,000 to about 40,000 daltons, about 10,000 to about 25,000 daltons, about 15,000 to about 25,000 daltons, about 20,000 to about 25,000 daltons, about 10,000 to about 20,000 daltons, about 15,000 to about 20,000 daltons, or about 10,000 to about 55,000 daltons. In some further embodiments, POLY has a weight average molecular weight selected from the group consisting of about 2,000 daltons (˜45), about 5,000 daltons (˜113), or about 10,000 daltons (˜227), or about 15,000 daltons (˜340), or about 20,000 daltons (˜454), or about 25,000 daltons (˜568), or about 30,000 daltons (˜681), or about 40,000 daltons (˜909), or about 45,000 daltons (˜1022), or about 50,000 daltons (˜1136), or about 60,000 daltons (˜1364), or about 65,000 daltons (˜1477), or about 70,000 daltons (˜1591), or about 75,000 daltons (˜1705), or about 80,000 daltons (˜1818), or about 90,000 daltons (˜2045), or about 100,000 daltons (˜2273) or greater (where, when POLY is polyethylene glycol, the number in parenthesis is the approximate number of ethylene glycol repeating units). Where POLY is a branched polymer, the weight average molecular weight as described above may refer to the total weight average molecular weight of the polymer portion of the conjugate or may refer to the weight average molecular weight of one of the polymer “arms”. For example, where POLY is a branched polymer comprising two or more polymer arms comprising repeating PEG units, the weight average molecular weight may refer to the overall weight of the branched polymer, or the weight average molecular weight of an individual PEG portion on each arm, and can readily be determined by context.

In one embodiment, POLY comprises a branched structure:

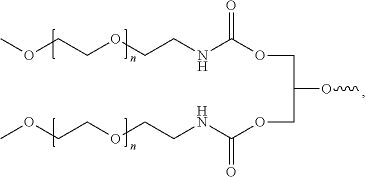

where each n is independently an integer ranging from about 45 to about 2273. In other embodiments, n is independently an integer ranging from about 45 to about 1818, from about 45 to about 909, from about 45 to about 455, from about 45 to about 341, from about 45 to about 227, or from about 45 to about 114. In some specific embodiments, n is independently selected from about 45, about 114, about 227, about 341, about 455, about 681, about 909, about 1136, about 1364, about 1818, and about 2273.

In some embodiments, the IFN-γ mutein conjugate has the structure:

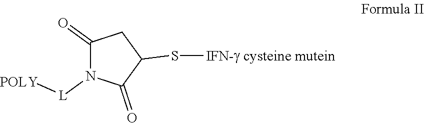

wherein IFN-γ is a cysteine mutein of IFN-γ, L, when present, is a linker as described herein, typically comprising one or more atoms selected from carbon, nitrogen, sulfur, oxygen, and combinations thereof, POLY is a water-soluble, non-peptidic polymer as described herein, and —S— is a sulfur atom of the IFN-γ mutein cysteine.

In even further embodiments, the IFN-γ mutein conjugate has the structure:

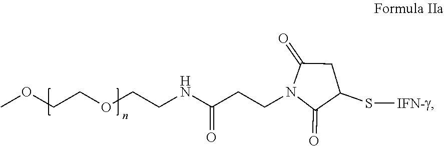

where IFN-γ is a cysteine modified mutein. The —S—in the structure, prior to conjugation, represents a thiol group of a cysteine that has been substituted or inserted into the IFN-γ molecule. In one or more embodiments, n is an integer having a value that corresponds to a polyethylene glycol polymer having a weight average molecular weight selected from the group consisting of about 2,000 daltons (n is ˜45), about 5,000 daltons (n is ˜113), about 10,000 daltons (n is ˜227), or about 15,000 daltons (n is ˜340), about 20,000 daltons (n is ˜454), about 25,000 daltons (n is ˜568), about 30,000 daltons (n is ˜681), about 40,000 daltons (n is ˜909), about 50,000 daltons (n is ˜1136), about 60,000 daltons (n is ˜1364), about 80,000 daltons (n is ˜1818), about 100,000 daltons (n is ˜2272), or greater.

In further embodiments, the IFN-γ conjugate has the structure of Formula IIb:

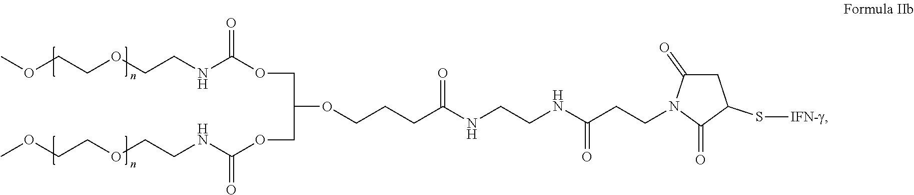

where IFN-γ is a cysteine modified mutein. The —S—in the structure, prior to conjugation, is a thiol group of a cysteine that has been substituted or inserted into the IFN-γ molecule. The “n” is an integer as described above In one or more embodiments, n is an integer having a value that corresponds to a polyethylene glycol polymer having a weight average molecular weight selected from the group consisting of about 2,000 daltons (n is ˜45), about 5,000 daltons (n is ˜113), about 10,000 daltons (n is ˜227), about 15,000 daltons (n is ˜340), about 20,000 daltons (n is ˜454), about 25,000 daltons (n is ˜568), about 30,000 daltons (n is ˜681), about 40,000 daltons (n is ˜909), about 50,000 daltons (n is ˜1136), about 60,000 daltons (n is ˜1364), or greater.

Further exemplary weight average molecular weights for the polyethylene glycol portion of the water-soluble, non-peptidic polymer for the conjugates as described herein, in addition to the foregoing, include about 11,000 daltons, about 12,000 daltons, about 13,000 daltons, about 14,000 daltons, about 15,000 daltons, about 20,000 daltons, about 22,500 daltons, about 25,000 daltons, about 35,000 daltons, about 40,000 daltons, about 45,000 daltons, about 50,000 daltons, about 55,000 daltons, about 60,000 daltons, about 65,000 daltons, about 70,000 daltons, about 75,000 daltons, about 80,000 daltons, about 90,000 daltons, about 100,000 daltons, about 110,000 daltons, or about 120,000 daltons.

It will be appreciated that the molecular weights for a branched polyethylene glycol polymers described herein, and the corresponding values for n, discussed herein may refer to the molecular weight for the polyethylene glycol portion of each polymer chain or the overall polyethylene glycol molecular weight for the conjugate.

In one or more preferred embodiments, the IFN-γ conjugate is selected from:

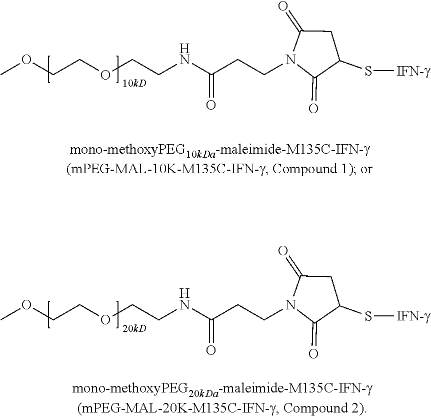

In one or more preferred embodiments, the IFN-γ conjugate is selected from:

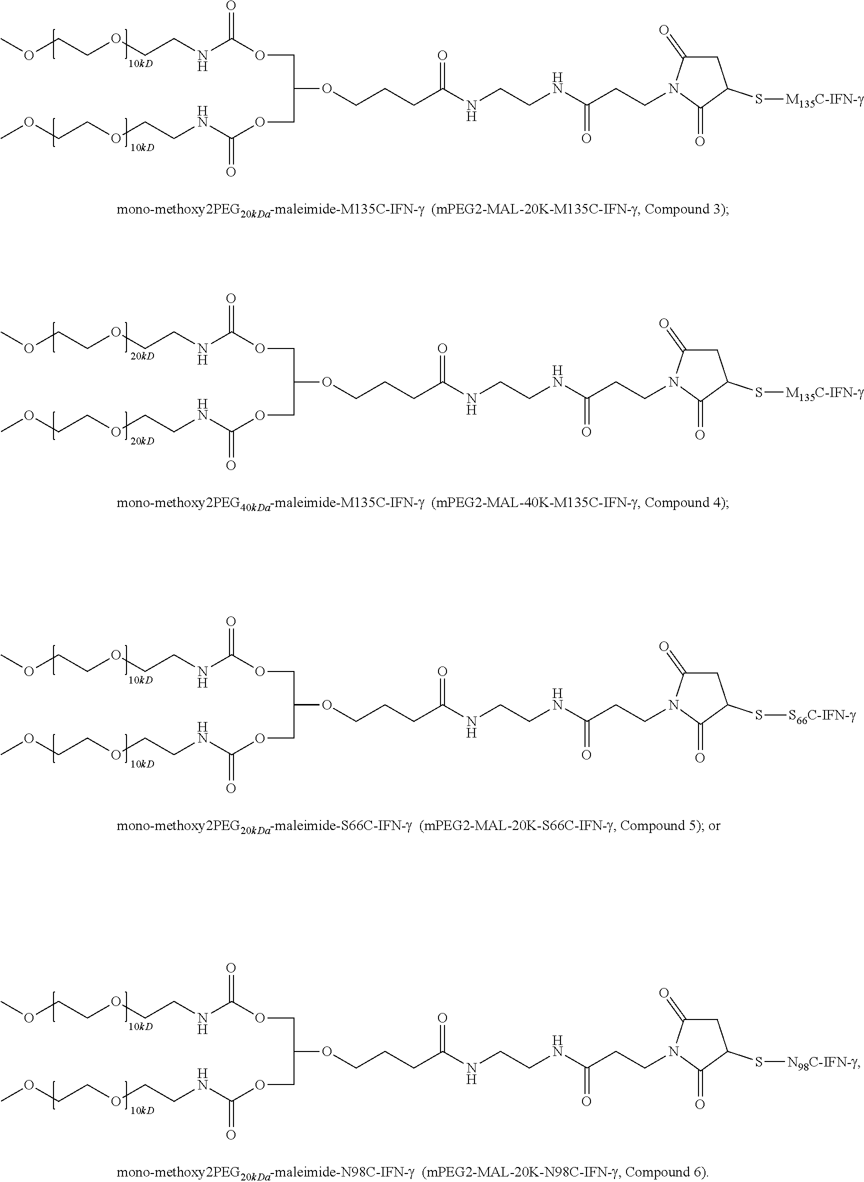

In some embodiments, conjugates may be formed using thiol-selective polymer reagents wherein a succinimide-group of the conjugate as described herein is converted to its more stable ring-opened succinamic acid form, also referred to herein as a ring-opened maleimide or ring-opened succinimide. The ring-opening is typically carried out by hydrolysis following the conjugation reaction to form a succinamic acid polymer IFN-γ cysteine mutein conjugate. Such conjugates may possess a diminished tendency towards hydrolysis as compared to other maleimide-derived conjugates and/or increased stability during storage and/or coupling, among having other advantages. Additionally, by virtue of providing hydrolyzed or “ring open” conjugates, conjugate compositions of greater homogeneity can be prepared. Such conjugates may further provide an enhanced safety profile when administered due to the lower incidence of thiol exchange and the resulting de-PEGylation. In particular, the ring-opened succinamic acid form of the conjugates may provide a lower acute toxicity than the corresponding ring-closed form of the conjugate.

Exemplary methods of preparing and using ring-open succinamic acid conjugates are described in PCT Publication No. WO 2004/060966, which is incorporated herein by reference. Briefly, a conjugate comprising a succinimide ring is exposed to an aqueous base under conditions effective to hydrolyze the succinimide group to a measurable degree, and preferably essentially to completion. The reaction conditions (e.g. temperature, pH, time of exposure, etc.) for the hydrolysis may be adjusted to achieve a desired extent of hydrolysis or ring opening. In embodiments, a conjugate comprising a succinimide group is treated under conditions effective to force open the succinimide ring to thereby form a ring-opened succinamic acid conjugate. In some embodiments, one or more of the pH, the temperature and/or the timing of the hydrolysis is adjusted to achieve a desirable rate of hydrolysis or ring opening. In some embodiments, the pH of the hydrolysis reaction is adjusted to about 6.0 to about 9.0, or about 7.0 to about 9.0, or about 7.0 to about 8.5. In specific embodiments, the pH is adjusted to about 6.0, about 6.5, about 7.0, about 7.5, about 8.0, about 8.5, or about 9.0. In embodiments, the hydrolysis reaction is carried out at a temperature of about 20° C. to about 37° C. In some embodiments, the hydrolysis reaction is carried out at a temperature of about 22° C. to about 25° C. In one particular embodiment, the hydrolysis reaction is carried out at room temperature. It will be appreciated that the temperature range may be adjusted based on the temperature sensitivity of the particular protein. The hydrolysis reaction may be carried out until all or substantially all the conjugate succinimide rings are opened. The timing of the hydrolysis reaction typically requires a few hours to overnight or 24 hours to reach completion. In one particular, but not limiting, embodiment, the hydrolysis reaction is carried out at room temperature, in a solution having a pH of about 8.5, and for a period of about 24 hours. An exemplary method of forming ring opened IFN-γ mutein conjugates is described in Example 10.

In embodiments, the hydrolysis reaction is carried out until at least about 15% to about 100% of the polymer-succinamic acid conjugate is formed. In some particular embodiments, about 20% to about 100%, about 25% to about 100%, about 50% to about 100%, about 75% to about 100%, about 80% to about 100%, about 90% to about 100%, about 95% to about 100%, about 20% to about 99%, about 25% to about 99%, about 50% to about 99%, about 75% to about 99%, about 80% to about 99%, about 90% to about 99%, about 20% to about 95%, about 25% to about 95%, about 50% to about 95%, about 75% to about 95%, about 80% to about 95%, about 90% to about 95%, about 20% to about 90%, about 25% to about 90%, about 50% to about 90%, about 75% to about 90%, about 80% to about 90%, about 20% to about 80%, about 25% to about 80%, about 50% to about 80%, or about 75% to about 80% of the polymer-succinamic acid conjugate is formed. In some preferred embodiments, the hydrolysis reaction is carried out until at least about 80%, at least about 90%, at least about 95%, at least about 99%, or at least about 100% of the succinimide groups in the conjugates are converted to the ring-opened form. Preferably, the hydrolysis reaction is carried out until complete, that is, until essentially all of the succinimide groups in the conjugates are converted to the ring-opened form.

In further embodiments, compositions described herein comprise at least about 50% to about 100% of the ring-opened form of the conjugates as compared to unhydrolyzed (ring-closed) conjugates in the composition. In additional embodiments, compositions described herein comprise at least about 60% to about 100%, about 70% to about 100%, about 75% to about 100%, about 80% to about 100%, about 90% to about 100%, about 95% to about 100%, about 99% to about 100%, about 60% to about 90%, about 70% to about 90%, about 75% to about 90%, or about 80% to about 90%, about 60% to about 95%, about 70% to about 95%, about 75% to about 95%, or about 80% to about 95%, about 90% to about 95% of the ring-opened form of the conjugates as compared to unhydrolyzed conjugates in the composition.

In even further embodiments, compositions as described herein comprise less than about 5% to less than about 99% by weight of unhydrolyzed (ring-closed) conjugates such as conjugates of Formula II in the composition as compared to the amount by weight of the ring-opened form of conjugates. Preferred are compositions comprising less than about 5%, about 10%, about 15%, about 20%, about 25%, about 30%, about 40%, or about 50% of the unhydrolyzed (ring-closed) conjugates in the composition as compared to the amount by weight of ring-opened form of conjugates. In even further embodiments, compositions as described herein comprise less than about 5% to less than about 99% mole percent of unhydrolyzed (ring-closed) conjugates in the composition as compared to the total mole percent of conjugates in the composition. Preferred are compositions comprising less than about 5%, about 10%, about 15%, about 20%, about 25%, about 30%, about 40%, or about 50% mole percent of the unhydrolyzed (ring-closed) conjugates in the composition as compared to the total mole percent of conjugates. In embodiments, the ring-closed conjugates referenced above have the ring closed structure of Formula II as described further above.

It will be appreciated that purification methods as known in the art may be used after the hydrolysis reaction in order to purify for the ring-open or ring-closed form of the conjugates. An exemplary method for purifying for the ring-open form of conjugates is described in Example 11. Further, the RC and RO conjugate forms may be separated after the hydrolysis reaction by any method as known in the art. An exemplary method for separating the RC and RO conjugate forms is described in Example 13.

In one embodiment, a conjugate formed with a ring-opened maleimide has a structure:

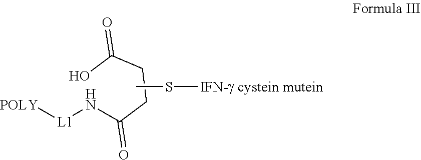

where IFN-γ is a cysteine mutein of IFN-γ, L1, when present, is a linker comprising one or more atoms selected from carbon, nitrogen, sulfur, oxygen, and combinations thereof. POLY is a water-soluble, non-peptidic polymer as described above, and —S—in the structure, prior to conjugation, is a thiol group of a cysteine that has been substituted or inserted into the IFN-γ molecule. In embodiments, L1 is a linker as described herein. In particular embodiments, the linker comprises one or more atoms selected from a carbon-carbon bond, an amide, a carbamate, an amine, an ether, and combinations thereof.

In an embodiment, a conjugate formed with a ring-opened maleimide has the structure:

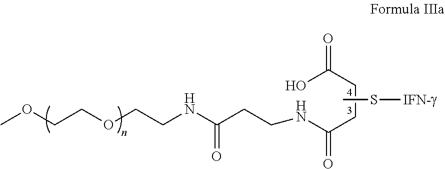

wherein IFN-γ is a cysteine mutein of IFN-γ, n for Formula IIIa is an integer from about 45 to about 1818, preferably from about 113 to about 1818, and —S—in the structure, prior to conjugation, is a thiol group of a cysteine that has been substituted or inserted into the IFN-γ molecule. Preferably in some embodiments, n is an integer having a value that corresponds to a polyethylene glycol polymer having a weight average molecular weight selected from the group consisting of about 2,000 daltons (n is ˜45), about 5,000 daltons (n is ˜113), about 10,000 daltons (n is ˜227), or about 15,000 daltons (n is ˜340), about 20,000 daltons (n is ˜454), about 25,000 daltons (n is ˜568), about 30,000 daltons (n is ˜681), about 40,000 daltons (n is ˜909), about 50,000 daltons (n is ˜1136), about 60,000 daltons (n is ˜1364), about 80,000 daltons (n is ˜1818), about 100,000 daltons (n is ˜2272), or greater.

In even further embodiments, a conjugate formed with a ring-opened maleimide has the structure:

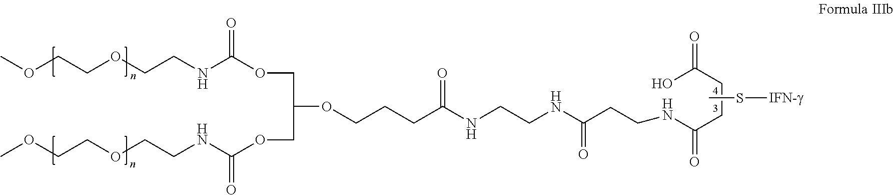

wherein IFN-γ is the cysteine mutein IFN-γ, n for Formula IIIb is independently an integer from about 45 to about 909, preferably from about 113 to about 909, and —S—in the structure, prior to conjugation, is a thiol group of a cysteine that has been substituted or inserted into the IFN-γ molecule. In some preferable embodiments, n is an integer having a value that corresponds to a polyethylene glycol polymer having a weight average molecular weight selected from the group consisting of about 2,000 daltons (n is ˜45), 5,000 daltons (n is ˜113), 10,000 daltons (n is ˜227), 15,000 daltons (n is ˜340), 20,000 daltons (n is ˜454), 25,000 daltons (n is ˜568), 30,000 daltons (n is ˜681), or 40,000 daltons (n is ˜909).

It will be appreciated that each of Formula III, Formula IIIa, and Formula IIIb encompasses structural isomers differing in the point of attachment of the nucleophilic group of the IFN-γ, that is at the 3-position or the 4-position of the succinamic acid as shown above. Use of ring-opened conjugates as described herein contemplates the use of the 3-isomer, the 4-isomer, or a mixture of 3-isomers and 4-isomers.

In one or more preferred embodiments, the IFN-γ conjugate is selected from:

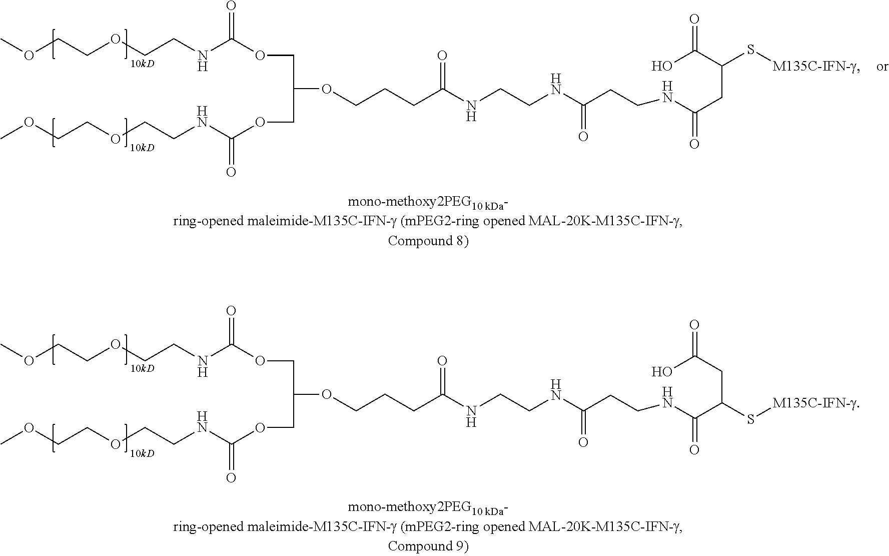

The conjugates described herein may be prepared in a variety of methods, and exemplary syntheses are provided in the examples which follow.

Optionally, the IFN-γ conjugate is comprised in a composition that comprises at least one pharmaceutically acceptable excipient. Exemplary excipients include, without limitation, those selected from the group consisting of carbohydrates, inorganic salts, antimicrobial agents, antioxidants, surfactants, buffers, acids, bases, amino acids, and combinations thereof.

It will be appreciated that the composition may comprise all or substantially all of a conjugate or a mixture of conjugates. In one exemplary embodiment, the composition comprises at least about 90% to about 99% of a particular conjugate as described herein. In some particular embodiments, at least about 75% to about 99% of the conjugates in the composition are the same conjugate. In further embodiments, at least about 80% to about 99%, at least about 80% to about 90%, or at least about 80% to about 95% of the conjugates in the composition are the same conjugate. In some particular embodiments, at least about 70%, at least about 75%, at least about 80%, at least about 85%, at least about 90%, at least about 95%, at least about 97%, at least about 99% or about 100% of the conjugates in the composition are the same conjugate. In another exemplary embodiment, the composition comprises at least about 90% to about 99% of a conjugate homodimer. In some particular embodiments, at least about 75% to about 99% of the conjugates in the composition are homodimers. In further embodiments, at least about 80% to about 99%, at least about 80% to about 90%, or at least about 80% to about 95% of the conjugates in the composition are homodimers. In some particular embodiments, at least about 70%, at least about 75%, at least about 80%, at least about 85%, at least about 90%, at least about 95%, at least about 97%, at least about 99% or about 100% of the conjugates in the composition are homodimers. In other embodiments, the composition comprises a mixture of homodimers and heterodimers.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, cyclodextrins, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The composition can also include an antimicrobial agent for preventing or deterring microbial growth. Nonlimiting examples of antimicrobial agents suitable for one or more embodiments of the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the conjugate or other components of the preparation. Suitable antioxidants for use in one or more embodiments of the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (both of which are available from BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; and IL-15 chelating agents, such as EDTA, zinc and other such suitable cations.

Acids or bases can be present as an excipient in the composition. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

One or more amino acids can be present as an excipient in the compositions described herein. Exemplary amino acids in this regard include arginine, lysine and glycine. Additional suitable pharmaceutically acceptable excipients include those described, for example, in the Handbook of Pharmaceutical Excipients, 9th ed., Sheskey, P. J., Ed., Pharmaceutical Press, 2020.

The instant IFN-γ mutein conjugates have been discovered to possess certain notable and advantageous features as described herein and as shown in the accompanying examples.

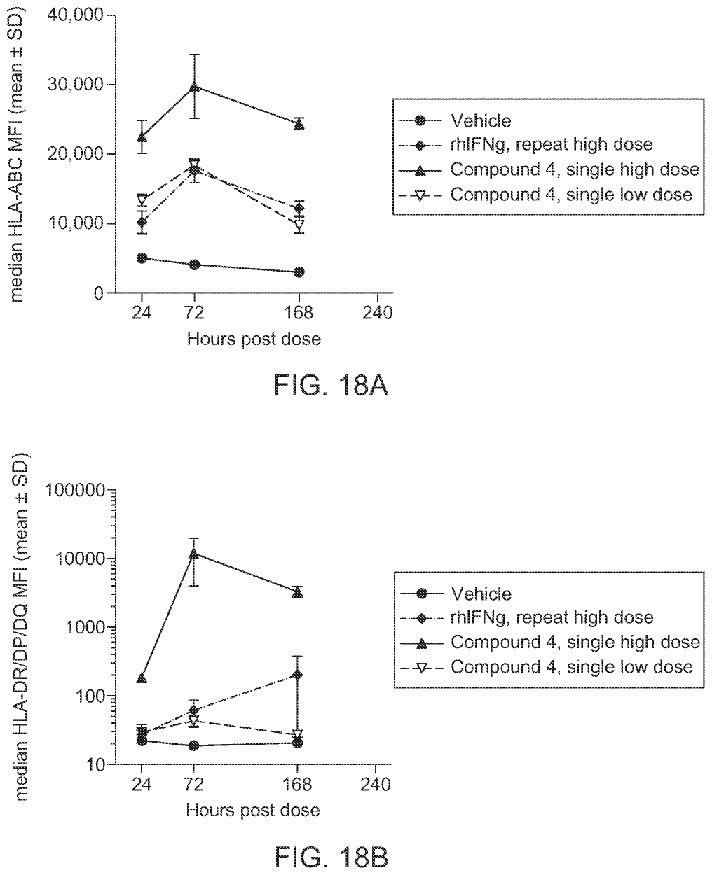

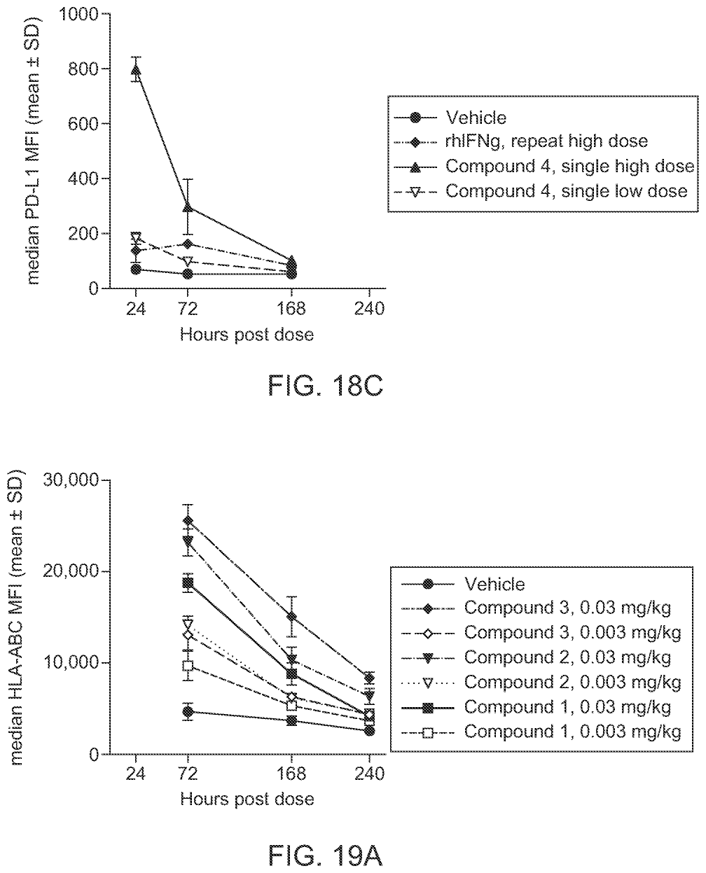

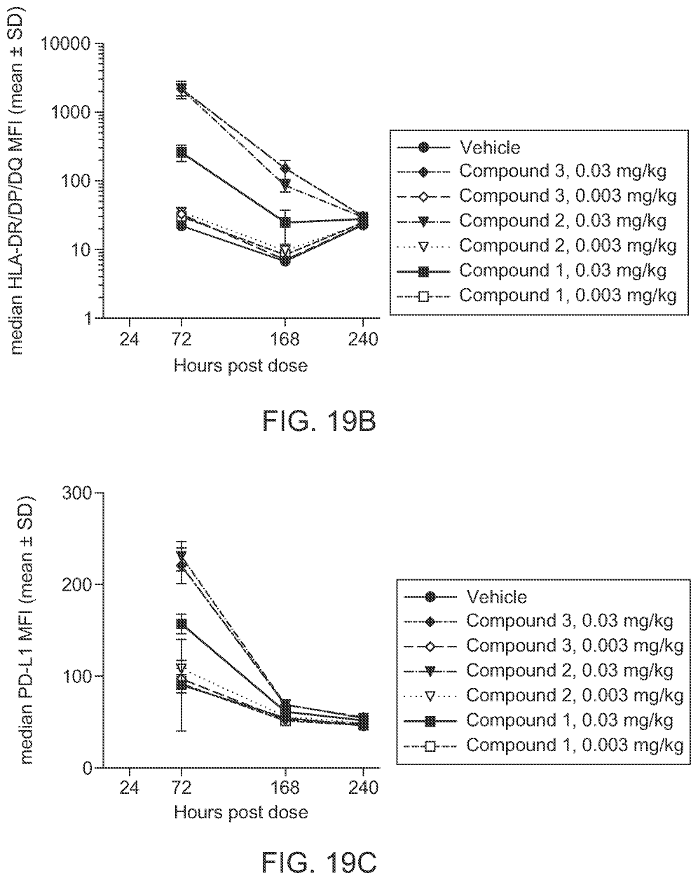

As described in Example 16, the in vitro activity of the illustrative conjugates (Compounds 1, 2, 3, and 4) induces IFN-γ signaling in huPBMCs, albeit at a lower potency than rhIFN-γ. As seen in Table 7, the conjugates were about 10 to 1000-fold less potent than the unmodified rhIFN-γ. The conjugates modified with a linear PEG exhibited higher potency than the conjugates modified with a branched PEG. Further experiments were conducted to investigate the downstream signaling in vivo of Compound 4 in a murine colorectal tumor (HT-29) model (Example 17). As shown in FIGS. 18A-18C and 19A-19C, at least Compound 4 was less potent as measured by phosphorylation of STAT1 and upregulation of PD-L1, however, the compound had similar potency in MHCI induction as compared to a non-polymer modified IFN-γ cysteine mutein and/or unmodified IFN-γ.

In some embodiments, the IFN-γ mutein conjugates described herein have a reduced signaling potency as compared to unmodified IFN-γ in order to provide one or more of the therapeutically desirable benefits including improved pharmacokinetic properties due to reduced target-mediated clearance; decreased induction of known negative feedback and counterregulatory mechanisms, and decreased receptor internalization and associated tachyphylaxis; and/or an expanded therapeutic index. In some embodiments, the instant IFN-γ mutein conjugates exhibit at least about a 10-fold to about a 1000-fold reduction in signaling potency. For example, in some embodiments, the instant IFN-γ mutein conjugates exhibit at least about a 10-fold reduction in potency, which may be represented by a 10-fold reduction in the half-maximal effective concentration (EC50) value. In general an EC50 value is calculated from a dose response curve as known in the art. In particular, the EC50 value as described herein is based on the measured concentration of the IFN-γ mutein conjugate that is necessary to cause half of the effect of the non-polymer modified IFN-γ mutein or the native IFN-γ as noted. In a particular embodiment, the instant IFN-γ mutein conjugates exhibit at least about a 10-fold reduction in EC50 value as measured by pSTAT1 (ng/mL, human PMBCs pSTAT1) when compared to a non-polymer modified IFN-γ cysteine mutein and/or unmodified IFN-γ. In or more related embodiments, the instant IFN-γ mutein conjugates exhibit at least about a 20-fold reduction in EC50 value (ng/mL, human PMBCs pSTAT1), or at least about a 25-fold reduction in EC50 value (ng/mL, human PMBCs pSTAT1), or at least about a 50-fold reduction in EC50 value (ng/mL, human PMBCs pSTAT1), or at least about a 75-fold reduction in EC50 value (ng/mL, human PMBCs pSTAT1), or at least about a 100-fold reduction in EC50 value (ng/mL, human PMBCs pSTAT1), or at least about a 200-fold reduction in EC50 value (ng/mL, human PMBCs pSTAT1), or at least about a 500-fold reduction in EC50 value (ng/mL, human PMBCs pSTAT1) or at least about a 750-fold reduction in EC50 value (ng/mL, human PMBCs pSTAT1), or at least about a 1000-fold reduction in EC50 value (ng/mL, human PMBCs pSTAT1) when compared to a non-polymer modified IFN-γ cysteine mutein and/or unmodified IFN-γ. It will be appreciated the EC50 values as recited herein reference the units, the experimental system (e.g. human PMBCs as described in Example 16) and the measured signal (e.g. STAT1 as described in Example 16).

As seen in Example 17, the IFN-γ mutein conjugates provide sustained MHC induction, which is essential for neoantigen presentation by tumor cells—leading to increased T cell recognition of tumor cells and increased tumor infiltration. In preferred embodiments, the instant IFN-γ mutein conjugates preferably provide a sustained, maintained, or even increase in expression (induction of expression) for MHCI and/or MHCII when compared to non-polymer modified IFN-γ (wild type IFN-γ and/or IFN-γ muteins), that is, the IFN-γ mutein conjugates as described herein provide higher expression of MHCI and/or MHCII and/or extended expression of MHCI and/or MHCII as compared to a non-polymer modified IFN-γ cysteine mutein and/or unmodified IFN-γ (wild type IFN-γ and/or IFN-γ muteins). In other words, the instant IFN-γ mutein conjugates preferably result in lowered reduction in MHCI induction when compared to non-polymer modified IFN-γ (wild type IFN-γ and/or IFN-γ muteins), that is, the IFN-γ mutein conjugates as described herein provide higher expression of MHCI and/or MHCII as well as extended expression of MHCI and/or MHCII as compared to a non-polymer modified IFN-γ cysteine mutein and/or unmodified IFN-γ (wild type IFN-γ and/or IFN-γ muteins), or at least a minimal reduction or decrease in the decrease in expression. In some additional embodiments, the IFN-γ mutein conjugates described herein have no more than about a 5-fold reduction in induction of MHCI induction as measured by EC50 value (ng/mL, HT-29 MHCI) when compared to a non-polymer modified IFN-γ cysteine mutein and/or unmodified IFN-γ. For example, in one or more related embodiments, the IFN-γ mutein conjugates have no more than about a 4.5-fold reduction in EC50 value (ng/mL, HT-29 MHCI), or no more than about a 4-fold reduction in EC50 value (ng/mL, HT-29 MHCI), or no more than about a 3.5-fold reduction in EC50 value (ng/mL, HT-29 MHCI), or no more than about a 3-fold reduction in EC50 value (ng/mL, HT-29 MHCI), or no more than about a 2.5-fold reduction in EC50 value (ng/mL, HT-29 MHCI), or no more than about a 2-fold reduction in EC50 value (ng/mL, HT-29 MHCI), or even no more than about a 1.5-fold reduction in EC50 value (ng/mL, HT-29 MHCI), or even no more than about a 3-fold reduction in EC50 value (ng/mL, HT-29 MHCI) when compared to a non-polymer modified IFN-γ cysteine mutein and/or unmodified IFN-γ (wild type IFN-γ and/or IFN-γ muteins). In further embodiments, the IFN-γ mutein conjugates have no more than about a 4.5-fold reduction in EC50 value (ng/mL, HT-29 MHCII), or no more than about a 4-fold reduction in EC50 value (ng/mL, HT-29 MHCII), or no more than about a 3.5-fold reduction in EC50 value (ng/mL, HT-29 MHCII), or no more than about a 3-fold reduction in EC50 value (ng/mL, HT-29 MHCII), or no more than about a 2.5-fold reduction in EC50 value (ng/mL, HT-29 MHCII), or no more than about a 2-fold reduction in EC50 value (ng/mL, HT-29 MHCII), or even no more than about a 1.5-fold reduction in EC50 value (ng/mL, HT-29 MHCI), or even no more than about a 3-fold reduction in EC50 value (ng/mL, HT-29 MHCII) when compared to a non-polymer modified IFN-γ cysteine mutein and/or unmodified IFN-γ (wild type IFN-γ and/or IFN-γ muteins).

### Methods of Treatment

In a further aspect, provided is a method for reducing heparin binding to IFN-γ by administering, alone or in combination with a further therapeutic agent or procedure, an IFN-γ mutein conjugate as described herein. See, for example, the results described in the accompanying examples herein.

In some embodiments, certain IFN-γ mutein conjugates as described herein are provided that are effective to mitigate or prevent the binding of heparin to the hIFN-γ conjugates thereby mitigating the inhibition of hIFN-γ by heparin. Heparin is thought to have several roles in the inhibition of the biological activity of IFN-γ. In particular, the heparin is thought to prevent binding of IFN-γ at least to the IFNGR1 receptor and the resulting lack of IFN-γ mediated biological activity. In addition, binding of soluble heparin to IFN-γ is thought to act as a “sink” and remove IFN-γ from the blood. To prevent heparin inhibition of IFN-γ, certain IFN-γ cysteine muteins of IFN-γ as described herein were designed to provide for strategic PEGylation of hIFN-γ in order to block heparin binding by virtue of inserting a cysteine in or near the heparin binding site of hIFN-γ (e.g. the C-terminus of the protein). It has been reported that a consensus sequence for heparin recognition involves the sequence KRKR (SEQ ID NO:10) (Lortat-Jacob (1996)). It was further reported that two clusters of basic residues that are located in the unstructured C-terminus of IFN-γ function as heparin binding sites (KTGKRKR (SEQ ID NO:11) and RGRR (SEQ ID NO:12)) (Sarrazin, et al., J. Biol. Chem., 280(45):37558-37564 (2005). As used herein, the C-terminus refers to the C-terminal 20-25 amino acids of the human IFN-γ protein. In a preferred embodiment, the IFN-γ mutein includes a cysteine substitution for one or more of amino acids 125-143 of SEQ ID NO:1 or one or more insertions within amino acids 121-143 of SEQ ID NO:1. In other embodiments, the IFN-γ mutein includes one or more cysteine substitution or insertion within 1-10 amino acids of the heparin recognition sequence KRKR (SEQ ID NO. 10) at either end. In some preferred embodiments, the IFN-γ mutein includes one or more cysteine substitution or insertion within 1-5 amino acids of the heparin recognition sequence KRKR (SEQ ID NO. 10) at either end. PEGylation at this site significantly reduces the ability of heparin to bind IFN-γ due to steric hindrance at the heparin binding site. Accordingly, strategic PEGylation of hIFN-γ mitigates the ability of heparin to reduce binding of IFN-γ to its receptor. As noted above, hIFN-γ has direct effect in IFN-γ-induced apoptosis. This effect can be almost totally inhibited in the presence of heparin. In embodiments, use of hIFN-γ mutein conjugates prevents the inhibitory effects of heparin binding and provides increased IFN-γ-induced apoptosis as compared to unmodified hIFN-γ or hIFN-γ conjugates where a water-soluble polymer is attached to hIFN-γ at a point distal from the heparin binding site of hIFN-γ even in the presence of heparin.

In certain embodiments, the present treatment is effective to decrease heparin binding to the hIFN-γ mutein conjugates. In embodiments, the present treatment is effective to decrease heparin binding (Kᵢ) by at least about 5%, 10%, 15%, 20%, 25%, 30%, 40%, 50%, 60%, 70%, 75%, 80%, 90% or 100% when compared to heparin binding to IFN-γ or an unmodified mutein of IFN-γ.

In some further embodiments, the IFN-γ mutein conjugates exhibit at least about a 1.5 fold reduction in heparin binding (Kᵢ) when compared to IFN-γ or an unmodified mutein of IFN-γ. That is to say, in some related embodiments, the IFN-γ mutein conjugates exhibit at least about a 1.6 fold reduction in heparin binding (Kᵢ), or exhibit at least about a 1.7 fold reduction in heparin binding (Kᵢ), or exhibit at least about a 1.8 fold reduction in heparin binding (Kᵢ), or exhibit at least about a 1.9 fold reduction in heparin binding (Kᵢ), or exhibit at least about a 2.0 fold reduction in heparin binding (Kᵢ), or exhibit at least than about a 2.4 fold reduction in heparin binding (Kᵢ), or even exhibit at least than about a 2.5 fold reduction in heparin binding (Kᵢ) when compared to IFN-γ or an unmodified mutein of IFN-γ. In some further embodiments, the IFN-γ mutein conjugates exhibit at least about a 1.5 to about 2.5 fold reduction in heparin binding (Kᵢ). The inhibition constant (Kᵢ) of an IFN-γ mutein conjugate may be calculated from comparing a heparin binding in a binding assay as described in Example 23 for the IFN-γ mutein conjugate as compared to heparin binding of a non-polymer modified IFN-γ mutein or an unmodified IFN-γ.

As described in Example 23, Compounds 1 and 2 had reduced heparin binding (Kᵢ) as compared to the unmodified IFN-γ mutein. The reduction in heparin binding (Kᵢ) was 2.4 and 1.6 fold lower, respectively, for the linear PEG conjugates as compared to the unmodified IFN-γ mutein.

In certain embodiments, the present treatment is effective to decrease heparin induced inhibition of IFN-γ binding. In embodiments, the present treatment is effective to decrease heparin induced inhibition by at least about 5%, 10%, 15%, 20%, 25%, 30%, 40%, 50%, 60%, 70%, 75%, 80%, 90% or 100% when compared to heparin binding to IFN-γ or an unmodified mutein of IFN-γ.

As described above, hIFN-γ induces MHCI and MHCII expression in both primary monocytes and tumor cells. However, this effect can be almost totally inhibited in the presence of heparin. In embodiments, use of hIFN-γ mutein conjugates prevents the inhibitory effects of heparin binding and provides increased IFN-γ-induced MHC expression in primary monocytes and/or tumor cells as compared to unmodified hIFN-γ or hIFN-γ conjugates where a water-soluble polymer is attached to hIFN-γ at a point distal from the heparin binding site of hIFN-γ even in the presence of heparin.

The ability to mitigate the inhibitory effects of heparin and other GAGs in the tumor microenvironment provides a direct and indirect (immunological) mode of action for IFN-γ that could live up to its expected anti-tumor properties.

In preferred embodiments, the IFN-γ muteins for use herein include cysteine mutations near or within the heparin binding domain of IFN-γ. Mutations can be selected based on proximity to the heparin binding domain and/or by selection of amino acids for substitution that have similar biochemical properties as cysteine.

The poly(ethylene glycol) conjugation site can also influence the heparin binding strength of the conjugate. Conjugation of the poly(ethylene glycol) inside or proximal to the hIFN-γ heparin binding domain decreases the heparin binding strength of the conjugate. Conjugation of the poly(ethylene glycol) to sites distal from the hIFN-γ heparin binding domain reduces the disruption of heparin binding strength of the conjugate. As described in Example 23, hIFN-γ muteins were produced having a cysteine substitution introduced at different positions relative to the heparin binding domain, e.g. hIFN-γ-M135C where the cysteine was within the heparin binding domain. In some embodiments, the degree of PEGylation of the IFN-γ mutein conjugate is generally inversely related to the heparin binding strength of the conjugate. Generally, as the degree of PEGylation increases, the heparin binding strength decreases. In further embodiments, the molecular weight of the poly(ethylene glycol) is inversely related to the heparin binding strength to the IFN-γ mutein conjugate. Thus, as the molecular weight used in preparing the conjugate increases, the heparin binding strength decreases. Selection of poly(ethylene glycol) architecture may also be used to modulate heparin binding strength. In general, branched architecture decreases heparin binding strength significantly more than linear architecture, even given equal poly(ethylene glycol) molecular weight.

In an additional aspect, provided is a method for reducing potency of IFN-γ and/or reducing the binding affinity of the molecule to IFNGR1 by administering, alone or in combination with a further therapeutic agent or procedure, an IFN-γ mutein conjugate as described herein. See, for example, the results described in the accompanying examples herein.

The IFNGR is a heterodimeric receptor consisting of IFN-γ Receptor Chain 1 (IFNGR1) and IFN-γ Receptor Chain 2 (IFNGR2). The IFNGR1 provides binding affinity and IFNGR2 is involved in signal transduction (Crisafulli et al., BioTechniques, 45(1):101-102 (2008)). Each IFN-γ homodimer binds to a tetrameric receptor structure consisting of two IFNGR1 and two IFNGR2 molecules with the complete complex comprising each IFN-γ monomer of the homodimer bound to an IFNGR1 and an IFNGR2. Both the amino and carboxyl termini of IFN-γ are required for binding to the IFNGR and the resulting biological response (Alspach, ibid.). It has been shown that IFN-γ initially binds to endothelial cells by interacting between the basic amino acids within the carboxyterminal (C-terminal) region and the IFNGR1 (Fluhr (2011)). Binding of IFN-γ to its receptor induces oligomerization of the receptor via trans-phosphorylation leading to the recruitment and activation of the Janus kinases 1 and 2 (JAK1 and JAK2) and the resulting activation of the signal transducer and activator of transcription 1 (STAT1)—the JAK-STAT signaling pathway. By reducing binding affinity of IFN-γ to IFNGR1, the signaling potency of IFN-γ is reduced, which is expected to provide improved pharmacokinetic properties due to reduced target-mediated clearance; decreased induction of known negative feedback and counterregulatory mechanisms, decreased receptor internalization and associated tachyphylaxis; decreased toxicity including acute toxicity, and/or an expanded therapeutic index.

In some embodiments, the IFN-γ mutein conjugates exhibits at least about a 5% reduction in IFNGR1 binding (equilibrium dissociation constant, K_{D}) when compared to unconjugated IFN-γ or other IFN-γ muteins, e.g., when measured using a technique suitable for determining IFNGR1 binding, such as, for example surface plasmon resonance (SPR). In some related embodiments, the IFN-γ mutein conjugates exhibits at least about a 10% reduction in IFNGR1 binding (K_{D}), exhibits at least about a 15% reduction in IFNGR1 binding (K_{D}), or exhibits at least about a 25% reduction in IFNGR1 binding (K_{D}), or exhibits at least about a 50% reduction in IFNGR1 binding (K_{D}), or even exhibits at least about a 75% reduction in IFNGR1 binding (K_{D}), or higher, when compared to unconjugated IFN-γ or other IFN-γ muteins. In some other embodiments, the IFN-γ mutein conjugates exhibits at least about a 1.0 fold reduction in IFNGR1 binding (K_{D}), or exhibits at least about a 1.0 fold reduction in IFNGR1 binding (K_{D}), exhibits at least about a 1.5 fold reduction in IFNGR1 binding (K_{D}), exhibits at least about a 2.0 fold reduction in IFNGR1 binding (K_{D}), exhibits at least about a 2.5 fold reduction in IFNGR1 binding (K_{D}), exhibits at least about a 5.0 fold reduction in IFNGR1 binding (K_{D}), exhibits at least about a 10.0 fold reduction in IFNGR1 binding (K_{D}), or exhibits at least about a 2.0 fold reduction in IFNGR1 binding (K_{D}). The K_{D} may be calculated from the ratio between the kinetic on-rate (kₒₙ) and the kinetic of-rate (k_{off}) using the formula K_{D}=kₒₙ/k_{off} as described in Example 19.

In a yet further aspect, provided is a method for treating a subject afflicted with a disease or indication treatable by administration of IFN-γ, such as a cancer or a tumor. The IFN-γ mutein conjugates may be used in as first line treatment, second line treatment, or third line treatment as appropriate and described herein. The method comprises administering, alone or in combination with a further therapeutic agent or procedure, an IFN-γ mutein conjugate as described herein. See, for example, the results described in the accompanying examples herein.

In one embodiment, the disease or indication is a cancer. In some embodiments, the cancer is a solid cancer. In other embodiments, the cancer is a liquid cancer. In further embodiments, the cancer is selected from small cell lung cancer (SCLC), non-small cell lung cancer (NSCLC), adrenocortical carcinoma, sarcomas including myxoid/round cell liposarcoma, synovial sarcoma, and gliosarcoma, fallopian tube cancer, ovarian cancer, renal cell carcinoma (RCC), colorectal cancer, microsatellite instability-high cancers (MSI-H/dMMR) including MSI-H/dMMR colorectal cancer, primary peritoneal cancer, breast cancer including triple-negative breast cancer (TNBC), classic Hodgkin lymphoma, gastric cancer, cervical cancer, primary mediastinal B-cell lymphoma (PMBCL), hepatocellular carcinoma (HCC), Merkel cell carcinoma (MCC), esophageal squamous cell cancer, cutaneous squamous cell carcinoma (cSCC), head and neck squamous cell cancer (HNSCC), bladder cancer including non-muscle invasive bladder cancer (NMIBC), urothelial carcinoma, glioblastoma, melanoma including malignant melanoma, and lymphomas such as T cell lymphomas including, but not limited to, mycosis fungoides and Sézary syndrome.

Notable findings associated with therapy using the IFN-γ conjugates as described herein are highlighted below and further detailed in the illustrative examples included herein.

In some embodiments, administration of IFN-γ conjugates as described herein is effective to provide an increase in the duration of survival of a subject over treatment of unmodified IFN-γ. In some other embodiments, administration of the IFN-γ conjugates as described herein is effective to provide an increase in the duration of survival of the subject over conventional treatment. In one or more embodiments, the duration of survival is increased by at least about 1, 2, 3, 4, 5, 6, 7, 8, 9, 10, 11, 12, 18, 24 months or longer when compared to a subject treated with unmodified IFN-γ or a conventional therapy for the same indication. In some embodiments, the duration of survival is at least about 1, 2, 3, 4, 5 years or longer.

In some embodiments, the present treatment is effective to increase the progression-free survival (survival without substantial progression of the disease being treated) of the subject. In embodiments, the progression-free survival is increased by at least about 1, 2, 3, 4, 5, 6, 7, 8, 9, 10, 11, 12, 18, 24 months or longer when compared to a subject treated with IFN-γ or a conventional therapy for the same indication. In some embodiments, the progression-free survival is at least about 1, 2, 3, 4, 5 years or longer.

An IFN-γ conjugate in accordance with the present disclosure may also be administered to a subject to treat an infection including, but not limited to bacterial infections including, but not limited to, mycobacterial infection, tuberculosis, meningitis including cryptococcal meningitis Streptococcal infections, viral infections including hepatitis such as chronic hepatitis B (HBV) and C (HCV), human immunodeficiency virus (HIV) infection or acquired immunodeficiency syndrome (AIDS), uveitis, and fungal infections such as Candidemia.

In further embodiments an IFN-γ conjugate in accordance with the present disclosure may also be administered to a subject in order to treat idiopathic pulmonary fibrosis, cystic fibrosis, multiple sclerosis, Crohn's Disease, leukocyte adhesion deficiency type I (LAD 1), colitis, rheumatoid arthritis, lupus, chronic granulomatous disease, osteopetrosis, scleroderma, Friedreich's ataxia, macular disease, myelodysplastic syndrome (MDS), and asthma.

In certain embodiments, the present treatment is effective to increase the response rate of subjects treated with the IFN-γ conjugates. In embodiments, the present treatment is effective to increase the response of treated subjects by at least about 5%, 10%, 15%, 20%, 25%, 30%, 40%, 50%, 60%, 70%, 75%, 80%, 90% or 100% when compared to a subject treated with IFN-γ or a conventional therapy for the same indication.

In some preferred embodiments, the IFN-γ conjugate is administered at a therapeutically effective dose. A therapeutically effective dose can be determined experimentally by repeated administration of increasing amounts of the IFN-γ conjugate in order to determine an amount that produces a clinically desired endpoint as described herein. Generally, a therapeutically effective amount will range from about 0.1 μg/m² to about 1000 μg/m² per administration, or about 25 μg/m² to about 1000 μg/m² per administration, inclusive. In further embodiments, the therapeutically effective amount ranges from about 0.1 μg/m² to about 500 μg/m², about 0.1 μg/m² to about 250 μg/m², about 0.1 μg/m² to about 100 μg/m², about 0.1 μg/m² to about 50 μg/m², about 0.1 μg/m² to about 25 μg/m², about 0.1 μg/m² to about 10 μg/m², about 0.1 μg/m² to 1 μg/m², about 1 μg/m² to about 500 μg/m², about 1 μg/m² to about 250 μg/m², about 1 μg/m² to about 100 μg/m², about 1 μg/m² to about 50 μg/m², about 1 μg/m² to about 25 μg/m², about 1 μg/m² to about 10 μg/m², about 10 μg/m² to about 500 μg/m², about 10 μg/m² to about 250 μg/m², about 10 μg/m² to about 100 μg/m², about 10 μg/m² to about 50 μg/m², about 10 μg/m² to about 25 μg/m², about 25 μg/m² to about 100 μg/m², from about 25 μg/m² to about 75 μg/m², from about 25 μg/m² to about 50 μg/m², from about 25 μg/m² to about 100 μg/m², from about 25 μg/m² to about 150 μg/m², from about 25 μg/m² to about 250 μg/m², from about 25 μg/m² to about 500 μg/m², from about 25 μg/m² to about 750 μg/m², from about 25 μg/m² to about 1000 μg/m², from about 50 μg/m² to about 150 μg/m², from about 50 μg/m² to about 100 μg/m², from about 50 μg/m² to about 75 μg/m², from about 50 μg/m² to about 100 μg/m², from about 50 μg/m² to about 150 μg/m², from about 50 μg/m² to about 250 μg/m², from about 50 μg/m² to about 500 μg/m², from about 50 μg/m² to about 750 μg/m², from about 50 μg/m² to about 1000 μg/m², from about 75 μg/m² to about 150 μg/m², from about 75 μg/m² to about 1000 μg/m², from about 100 μg/m² to about 1000 μg/m², from about 200 μg/m² to about 1000 μg/m², from about 300 μg/m² to about 1000 μg/m², from about 400 μg/m² to about 1000 μg/m², from about 500 μg/m² to about 1000 μg/m², from about 600 μg/m² to about 1000 μg/m², from about 700 μg/m² to about 1000 μg/m², from about 800 μg/m² to about 1000 μg/m², or from about 900 μg/m² to about 1000 μg/m² per administration. In some particular, but not limiting embodiments, the IFN-γ conjugate is administered at a dose of about 0.1 μg/m², 1.0 μg/m², 19 μg/m², 25 μg/m², 50 μg/m², 75 μg/m², 100 μg/m², 150 μg/m², 200 μg/m², 250 μg/m², 300 μg/m², 400 μg/m², 500 μg/m², 600 μg/m², 700 μg/m², 800 μg/m², 900 μg/m², or 1000 μg/m² per administration. In other embodiments, a therapeutically effective amount ranges from about 1.0 μg/kg to about 10 μg/kg per administration. In additional embodiments, the therapeutically effective amount ranges from about 1.0 μg/kg to about 5 μg/kg, from about 1.0 μg/kg to about 2 μg/kg, from about 1.0 μg/kg to about 1.5 μg/kg, from about 1.5 μg/kg to about 10 μg/kg, from about 1.5 μg/kg to about 5 μg/kg, or from about from about 5.0 μg/kg to about 10 μg/kg per administration. In some specific, but not limiting embodiments, a therapeutically effective amount is about 1.0 μg/kg, about 1.5 μg/kg, about 5 μg/kg, or about 10 μg/kg per administration. In some further, but not limiting embodiments, a therapeutically effective amount is about 0.1 μg, about 1.0 μg, about 10 μg, about 25 μg, about 50 μg, about 75 μg, about 100 μg, about 200 μg, about 250 μg, about 500 μg, about 600 μg, about 700 μg, about 750 μg, about 800 μg, about 900 μg, or about 1000 μg per administration. It will be appreciated that the therapeutically effective amount of the conjugate may be any dose as approved by a governmental regulatory agency for an IFN-γ protein, peptide, or fragment thereof. With reference to the doses referenced in the examples herein, one of ordinary skill in the art could convert the animal doses (e.g. mouse) to a corresponding dose in humans using conversions as known in the art (e.g. Nair et al., J. Basic and Clin. Pharmacy (2016) 7:27-31).

It will be appreciated that the doses for the IFN-γ conjugate as described above may refer to either of the compound or the IFN-γ protein equivalent. In preferred embodiments, the doses refer to the IFN-γ protein equivalents.

It will be appreciated that the actual dose of the conjugate to be administered will vary depending upon the age, weight, body surface area, and general condition of the subject as well as the severity of the condition being treated, the judgment of the health care professional, and conjugate being administered. Therapeutically effective amounts are known to those skilled in the art and/or are described in the pertinent reference texts and literature.

The dose of the conjugate (preferably provided as part of a pharmaceutical composition or preparation) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration once daily, three times weekly, twice weekly, once weekly (q1w), twice monthly (e.g. q/14 days), once monthly (e.g. q/30 days or 31 days, q/28 days or q/21 days), and any combination thereof. It will be appreciated that the dosing schedule may be adjusted as needed, e.g. administration once weekly for a period of time and then adjusted to a shorter or longer schedule as needed. Once a desired clinical endpoint has been achieved, dosing of the composition is halted or reduced. In some embodiments, the unit dose of any given conjugate may be administered once to provide sustained effect. A given dose can be periodically administered up until, for example, the clinician determines an appropriate endpoint (e.g., cure, regression, partial regression, and so forth) is achieved. As noted above, chronic IFN-γ exposure may lead to PD-L1 induction and a pro-tumor response. Accordingly, it may be beneficial to select a dosing frequency that allows for a rest period in order to prevent chronic IFN-γ signaling.

The IFN-γ conjugate may be administered by any suitable means as known in the art. In some embodiments, the IFN-γ conjugate may be administered parenterally which includes subcutaneous, intravenous, intra-arterial, intraperitoneal, intracardiac, intrathecal, and intramuscular injection, as well as infusion injections. Suitable formulation types for parenteral administration include ready-for-injection solutions, dry powders for combination with a solvent prior to use, suspensions ready for injection, dry insoluble compositions for combination with a vehicle prior to use, and emulsions and liquid concentrates for dilution prior to administration, among others. In some particular embodiments, the IFN-γ conjugate is provided in a formulation suitable for intravenous administration and is administered intravenously. In some other embodiments, the IFN-γ conjugate is provided in a formulation suitable for subcutaneous administration and is administered subcutaneously. Other modes of administration are also contemplated, such as pulmonary, nasal, buccal, rectal, sublingual, and transdermal.

### Combination Methods of Treatment

In some embodiments, the IFN-γ conjugates or compositions provided herein are administered in combination with another pharmacologically active agent or other suitable compound or other treatment.

Both adaptive immune resistance and acquired immune resistance are used by tumors to escape the immunological action of the immune system. IFN-γ signaling (or downstream targets of IFN-γ signaling) can be used by tumors to effect escape. Upregulation of PD-L1 occurs on both tumor cells and normal cells in response to IFN-γ signaling. Accordingly, the IFN-γ conjugates described herein provide synergistic interactions with T cell augmenting therapeutics such as checkpoint inhibitors that reduce tumor checkpoint ligand dependent T cell inactivation.

As described in Example 21, administration of the recombinant mouse IFN-γ (rmIFN-γ) conjugate (mono-mPEG2-ru-MAL-20K-rmIFN-γ) in combination with a mouse anti-PD-1 antibody or a mouse anti-PD-L1 antibody provided an increased efficacy in slowing tumor growth over single agent administration of any of the rmIFN-γ, the PD-1 antagonist, or the PD-L1 antagonist in a murine B16F10 melanoma tumor model. On day seven after treatment, administration of a rmIFN-γ conjugate in combination with either of a mouse anti-PD-1 antibody or a mouse anti-PD-L1 antibody more than doubled the tumor growth inhibition (TGI) percentage as compared to treatment with a vehicle.

In one preferred combination, the IFN-γ conjugates or compositions are administered in combination with at least one PD-1/PD-L1 axis inhibitor. The treatment methods provided herein comprise administering a PD-1/PD-L1 axis inhibitor, e.g., for immune checkpoint blockade. Administration of the PD-1/PD-L1 axis inhibitor is effective to, for example, enhance T cell cytolytic activity.

Various PD-1/PD-L1 axis inhibitors can be utilized and/or administered in accordance with the compositions, systems, combinations, and methods described herein, and the compositions, systems, combinations, and methods herein are not limited in this regard. Without being limited as to theory, it is believed that successful outcomes can be achieved via co-administration of a PD-1/PD-L1 axis inhibitor with IFN-γ to stimulate the desired T cell responses.

Illustrative PD-1/PD-L1 axis inhibitors include, but are not limited to, for example: atezolizumab (TECENTRIQ®, MPDL3280A, Roche Holding AG), avelumab (BAVENCIO®, MSB0010718C, Merck KGaA), durvalumab (IMFINZI®, AstraZeneca PLC), nivolumab (OPDIVO®, ONO-4538, BMS-936558, MDX1106, Bristol-Myers Squibb Company), pembrolizumab (KEYTRUDA®, MK-3475, lambrolizumab, Merck & Co., Inc.), BCD100 (BIOCAD Biopharmaceutical Company), BGB-A317 (BeiGene Ltd./Celgene Corporation), CBT-501 (CBT Pharmaceuticals), CBT-502 (CBT Pharmaceuticals), GLS-010 (Harbin Gloria Pharmaceuticals Co., Ltd.), 1B1308 (Innovent Biologics, Inc.), WBP3155 (CStone Pharmaceuticals Co., Ltd.), AMP-224 (GlaxoSmithKline plc), BI 754091 (Boehringer Ingelheim GmbH), BMS-936559 (Bristol-Myers Squibb Company), CA-170 (Aurigene Discovery Technologies), FAZ053 (Novartis AG), LY3300054 (Eli Lilly & Company), M7824 (Merck KGaA), MED10680 (AstraZeneca PLC), PDR001 (Novartis AG), PF-06801591 (Pfizer Inc.), REGN2810 (Regeneron Pharmaceuticals, Inc.), SHR-1210 (Incyte Corporation), TSR-042 (Tesaro, Inc.), AGEN2034 (Agenus Inc.), CX-072 (CytomX Therapeutics, Inc.), JNJ-63723283 (Johnson & Johnson), MGD013 (MacroGenics, Inc.), AN-2005 (Adlai Nortye), ANA011 (AnaptysBio, Inc.), ANB011 (AnaptysBio, Inc.), AUNP-12 (Pierre Fabre Medicament S.A.), BBI-801 (Sumitomo Dainippon Pharma Co., Ltd.), BION-004 (Aduro Biotech), CA-327 (Aurigene Discovery Technologies), CK-301 (Fortress Biotech, Inc.), ENUM 244C8 (Enumeral Biomedical Holdings, Inc.), FPT155 (Five Prime Therapeutics, Inc.), FS118 (F-star Alpha Ltd.), hAb21 (Stainwei Biotech, Inc.), J43 (Transgene S.A.), JTX-4014 (Jounce Therapeutics, Inc.), K_{D}033 (Kadmon Holdings, Inc.), KY-1003 (Kymab Ltd.), MCLA-134 (Merus B.V.), MCLA-145 (Merus B.V.), PRS-332 (Pieris AG), SHR-1316 (Atridia Pty Ltd.), STI-A1010 (Sorrento Therapeutics, Inc.), STI-A1014 (Sorrento Therapeutics, Inc.), STI-A1110 (Les Laboratoires Servier), XmAb20717 (Xencor, Inc.), and pidilizumab (CT-011, Medivation).

BGB-A317 (tislelizumab), under development by BeiGene Ltd., is a humanized IgG4, monoclonal antibody having an engineered Fc region (i.e., where the ability to bind Fc gamma receptor I has been specifically removed). BGB-A317 binds to PD-1 and inhibits the binding of PD-1 to PD-L1 and PD-L2.

In one or more embodiments, the PD-1/PD-L1 axis inhibitor is selected from atezolizumab, avelumab, durvalumab, nivolumab, pembrolizumab, and BGB-A317. It will be appreciated that one or more PD-1/PD-L1 axis inhibitors can be administered in the combination treatment methods provided herein.

In some embodiments, administration of an IFN-γ conjugate as described herein provides a TGI of at least about 50% to about 60% when compared to tumor growth after treatment with a vehicle. In some further embodiments, administration of an IFN-γ conjugate as described herein in combination with a PD-1 antagonist or a PD-L1 antagonist provides a TGI of at least about 50% to about 70% when compared to tumor growth after treatment with a vehicle. In yet other embodiments, administration of IFN-γ conjugate as described herein in combination with a PD-1 antagonist or a PD-L1 antagonist provides an increase in TGI of at least about 30% to about 45% when compared to TGI after treatment with the PD-1 antagonist or the PD-L1 antagonist as a single agent.

In another preferred combination, the IFN-γ conjugates or compositions as described herein are administered in combination with at least one antagonist of CTLA-4. In a specific embodiment, the IFN-γ conjugates or compositions are administered in combination with a CTLA-4 pathway-inhibiting amount of an anti-CTLA-4 antibody. One of ordinary skill in the art can determine how much a given anti-CTLA-4 antibody is sufficient to provide clinically relevant inhibition of the CTLA-4 pathway. For example, one of ordinary skill in the art can refer to the literature and/or administer a series of increasing amounts the anti-CTLA-4 antibody and determine which amount or amounts provide clinically relevant inhibition the CTLA-4 pathway. Anti-CTLA-4 antibodies are known and include tremelimumab and ipilimumab, for example.

In a further combination, IFN-γ conjugates or compositions as described herein are administered in combination with at least one antagonist of lymphocyte activation gene-3 (LAG-3). In a specific embodiment, the IFN-γ conjugates or compositions are administered in combination with a LAG-3-inhibiting amount of an anti-LAG-3 antibody. One of ordinary skill in the art can determine how much a given anti-LAG3 antibody is sufficient to provide clinically relevant inhibition of LAG-3. For example, one of ordinary skill in the art can refer to the literature and/or administer a series of increasing amounts the anti-LAG-3 antibody and determine which amount or amounts provide clinically relevant inhibition of LAG-3. Exemplary anti-LAG-3 antibodies include relatlimab (Opdualag® Bristol-Myers Squibb Company), Sym022 (Syrnphogen A/S), TSR-033 (Tesaro, Inc.), REGN3767 (Regeneron Pharmaceuticals, Inc.), ieramilimag (LAG525, Novartis), INCAGN2385-101 (Incyte Biosciences), favezelimib (Merck & Co.), and miptenalimab (BI 754111, Boehringer Ingelheim), In addition, a number of antagonistic bispecific antibodies targeting LAG-3 are known including MGDO13 (an anti-PD-1/LAG-3 antibody, MacroGenics), FS118 (an ant-LAG-3/PD-L1 antibody, F-Star Therapeutics, Inc.), and XmAb22841 (an anti-CTLA-4/LAG-3 antibody, Xencor, Inc.). In some embodiments, the IFN-γ conjugates are administered in combination with one or more LAG-3 antagonists and one or more PD-1 and/or PD-L1 antagonists as described above.

In yet a further combination, IFN-γ conjugates or compositions as described herein are administered in combination with at least one antagonist of T cell immunoreceptor with immunoglobulin and ITIM domain (TIGIT). In a specific embodiment, the IFN-γ conjugates or compositions are administered in combination with a TIGIT pathway-inhibiting amount of an anti-TIGIT antibody. One of ordinary skill in the art can determine how much a given anti-TIGIT antibody is sufficient to provide clinically relevant inhibition of the TIGIT pathway. For example, one of ordinary skill in the art can refer to the literature and/or administer a series of increasing amounts the anti-TIGIT antibody and determine which amount or amounts provide clinically relevant inhibition the TIGIT pathway. Anti-TIGIT antibodies are known and include tiragolumab (Genentech), BMS-986207 (Bristol-Myers Squibb Company), vibostolimab (MK-7684, Merck), EOS-448 (iTeos Therapeutics), domvanalimab (AB154, Arcus Biosciences), ociperlimab (BGB-A1217, (BeiGene), tamgiblimab (IBI-939, Innovent Bio), COM902 (Compugen), HLX53 (Shanghai Henlius Biotech, Inc.), and JS006 (Junshi Biosciences). In some embodiments, the IFN-γ conjugates are administered in combination with one or more TIGIT antagonists and one or more PD-1 and/or PD-L1 antagonists as described above.

By way of clarity, with regard to the sequence of administering, wherein the term “administering” is used in this instance to refer to delivery of either the IFN-γ conjugate or the secondary therapeutic agent concurrently or sequentially and in any order. Moreover, treatment of either component of the combination may comprise a single cycle of therapy or may comprise multiple cycles. That is to say, following administration of the IFN-γ conjugate and administration of the secondary therapeutic agent, additional rounds of therapy may include administration of the IFN-γ conjugate in combination with administration of the secondary agent, administration of the IFN-γ conjugate without further administration of the secondary agent, or administration of the secondary agent without further administration of the IFN-γ conjugate, or any combination of the above administrations.

In further embodiments, the IFN-γ conjugates or compositions provided herein are administered in combination with standard of care for treating a particular indication or condition. An exemplary standard of care is chemotherapy, e.g. platinum based chemotherapy, or radiation treatment for some cancers.

In a third aspect, a kit comprising the IFN-γ conjugates as described above is provided herein. The kit may further include instructions for use as well as, optionally, any medical supplies or devices as needed for administration of the IFN-γ conjugates.

It is to be understood that while the methods, combinations, kits, etc. have been described in conjunction with the preferred specific embodiments thereof, that the foregoing description as well as the examples that follow are intended to illustrate and not limit the scope of the disclosed methods, combinations, kits, etc. Other aspects, advantages, and modifications within the scope of this disclosure will be apparent to those of ordinary skill in the relevant art.

All articles, books, patents, and other publications referenced herein are hereby incorporated by reference in their entireties. In the event of an inconsistency between the teachings of this specification and the art incorporated by reference, the meaning of the teachings and definitions in this specification shall prevail (particularly with respect to terms used in the claims appended herein). For example, where the present application and a publication incorporated by reference defines the same term differently, the definition of the term shall be preserved within the teachings of the document from which the definition is located.

### EXAMPLES

It is to be understood that the foregoing description as well as the examples that follow are intended to illustrate and not limit the scope of the invention(s) provided herein. Other aspects, advantages and modifications will be apparent to those skilled in the art to which this disclosure pertains.

In the following examples, efforts have been made to ensure accuracy with respect to numbers used (e.g., amounts, temperatures, etc.) but some experimental error and deviation should be taken into account. Unless indicated otherwise, temperature is in degrees c. and pressure is at or near atmospheric pressure at sea level. Each of the following examples is considered to be instructive to one of ordinary skill in the art for carrying out one or more of the embodiments described herein.

### Materials and Methods

### SDS-PAGE Analysis

Samples were analyzed by sodium dodecyl sulfate-polyacrylamide gel electrophoresis (SDS-PAGE) using Invitrogen gel electrophoresis system (XCell SureLock Mini-Cell). Samples were mixed with sample buffer. Then, the prepared samples were loaded onto a NuPAGE Novex precast gel and run for approximately thirty minutes.

### RP-HPLC Analysis

Reversed-phase chromatography (RP-HPLC) analysis was performed on an Agilent 1200 HPLC system (Agilent). Samples were analyzed using a Poroshell 300SB—C3 column (2.1×75 mm, Agilent) at 60° C. The mobile phases are 0.1% TFA/H2O (A) and 0.1% TFA/CH3CN (B). The flow rate for the column was 0.5 ml/min. Eluted protein and PEG-protein conjugates were detected using UV at 215 nm or 280 nm.

### Example 1

### Design and Preparation of IFN-γ Cysteine Muteins

The native, human IFN-γ sequence does not contain any cysteine amino acids. Therefore, muteins were designed where one native residue of hIFN-γ was mutated to a cysteine residue to enable site specific conjugation of water-soluble, non-peptidic polymers using, e.g. thiol reactive poly(ethylene glycol) reagents such as maleimide active poly(ethylene glycol) molecules. The muteins prepared were derived from the amino acid sequence of IFN-γ (Uniprot accession number P01579, SEQ ID NO:3) from glutamine at position 24 to arginine at position 162 (SEQ ID NO:7). Initially, a methionine residue was fused to the N-terminus of the glutamine for translation initiation (SEQ ID NO:2). With reference to numbering of the muteins prepared herein, the N-terminal methionine is position 1.

To generate the IFN-γ-M135C sequence, the methionine at position 135 of the IFN-γ sequence was replaced with a cysteine. To generate the IFN-γ-S66C sequence, the serine at position 66 of the IFN-γ sequence was replaced with a cysteine. To generate the IFN-γ-N98C sequence, the asparagine at position 98 of the IFN-γ sequence was replaced with a cysteine.

The amino acid sequences of the muteins prepared are shown below with the cysteine residue highlighted in bold and underlined:

1. hIFN-γ S66C:
(SEQ ID NO: 4)
MQDPYVKEAE NLKKYFNAGH SDVADNGTLF LGILKNWKEE
SDRKIMQSQI VSFYFKLFKN FKDDQCIQKS VETIKEDMNV
KFFNSNKKKR DDFEKLTNYS VTDLNVQRKA IHELIQVMAE
LSPAAKTGKR KRSQMLFRGR
2. hIFN-γ N98C
(SEQ ID NO: 5)
MQDPYVKEAE NLKKYFNAGH SDVADNGTLF LGILKNWKEE
SDRKIMQSQI VSFYFKLFKN FKDDQSIQKS VETIKEDMNV
KFFNSNKKKR DDFEKLTCYS VTDLNVQRKA IHELIQVMAE
LSPAAKTGKR KRSQMLFRGR
3. hIFN-γ M135C:
(SEQ ID NO: 6)
MQDPYVKEAE NLKKYFNAGH SDVADNGTLF LGILKNWKEE
SDRKIMQSQI VSFYFKLFKN FKDDQSIQKS VETIKEDMNV
KFFNSNKKKR DDFEKLTNYS VTDLNVQRKA IHELIQVMAE
LSPAAKTGKR KRSQCLFRGR

Mutein Production: The nucleotide sequences encoding for the mutein polypeptides were generated using GeneGPS@technology (Atum, Newark, CA). The mutein nucleotide sequences were then inserted into the pD454-SR vector (Atum) under the control of the T7 promoter and lac operator for inducible expression in bacterial cells. The utilized plasmid also confers ampicillin resistance.

Mutein Expression: Plasmids containing the mutein expression cassettes were transformed into chemically competent BL21(DE3) E. coli cells (New England Biolabs). Selection of the clones containing the recombinant vector was based on ampicillin resistance. The selected clones were then grown in 2×YT media (Teknova) with 100 μg/mL ampicillin and glycerol stocks of these clones were created by adding 80% v/v glycerol to the cultures in a 1:1 volume ratio (40% glycerol final concentration).

To initiate a production culture in shake flasks, 5 mL inoculation cultures were seeded from the glycerol stocks and grown in 15 mL culture tubes for 12-18 hours at 37° C. and shaken at 220 rpm. The production runs were performed in 1 L cultures grown in 2.8 L shake flasks. Both the inoculation and production cultures were grown in 2×YT media (Teknova) with 100 μg/mL ampicillin. The production cultures were inoculated using three 5 mL inoculation cultures and the culture was grown at 37° C. and shaken at 220 rpm. Production of the IFN-γ muteins were induced by adding 1 mM isopropyl β-D-1-thiogalactopyranoside (IPTG) into the cultures when the culture OD600 reached 0.6. After induction, the cultures were kept at 37° C. and 220 rpm. The cultures were harvested 6-8 hours after induction. To harvest, the cultures were centrifuged at 9000 g for 40 minutes. The supernatant was discarded, and the pelleted cell paste was collected and stored at −80° C.

The IFN-γ muteins were expressed as inclusion bodies (IBs) in the bacterial cells. The frozen cell paste was thawed at room temperature and resuspended in 1×TE buffer containing 10 mM Tris pH 8 and 1 mM ethylenediaminetetraacetic acid (EDTA). The volume of 1×TE buffer used was 10 mL buffer per gram of wet cell paste. The resuspended cells were then lysed using an M-110P microfluidizer (Microfluidics). The resuspended cells were passed through the microfluidizer twice, each time with a ΔP of 10-25 kPsi. The IBs of the lysed cells were harvested by centrifuging the lysis mixture at 10-15 kg for 30-60 minutes. The supernatant was discarded, and the pelleted IBs were collected.

The IBs were then washed with three different buffers: (1^{st} wash) 50 mM Tris pH 8, 5 mM EDTA, 2% Triton X100, and 1% Tween 20, (2^{nd} wash) 50 mM Tris pH 8, 5 mM EDTA, and 1 M sodium chloride, and (3^{rd} wash) 50 mM Tris pH 8, 5 mM EDTA, and 20 mM sodium chloride. For each wash, the IBs were resuspended in the respective buffer, using 30-50 mL buffer volume per gram of IBs. The resuspended IBs were then harvested by centrifugating at 10-15 kg for 30-60 minutes.

Mutein Refolding and Purification: The protocol for refolding and purification of the IFN-γ muteins was adapted from Arora et al. (Journal of Biotechnology, 1996, 52:127-133). The washed IBs were resolubilized by resuspending the IBs in a buffer containing 6M guanidine hydrochloride, 100 mM Tris pH 8, 50 mM dithiothreitol (DTT) and 0.2 mM EDTA. The resuspended mixture was stirred and incubated at room temperature for 16-18 hours. The mixture was then clarified by centrifugation at 10-15 kg for 30-60 minutes. The supernatant containing the resolubilized protein was retained and the pellet was discarded.

Refolding of the IFN-γ muteins was initiated by flash diluting the resolubilized protein into a refolding buffer containing 0.5M arginine, 100 mM Tris pH 8, 0.2 mM EDTA, and 10 mM DTT. The DTT was added to reduce any oxidized cysteines and to maintain a reducing environment for refolding. Prior to use, the refolding buffer was chilled to 4° C. The solution containing the resolubilized protein was added into the refolding buffer as three separate boluses at 2 hour intervals. The volume of each bolus was 10 mL per liter of refolding buffer. After the third bolus, the solution was incubated without agitation at 4° C. for 48 hours. The solution was then dialyzed against 20 mM Tris pH 8, 100 mM urea, and 5 mM DTT for 48 hours with one buffer change at 24 hours. After dialysis, the solution was clarified using centrifugation at 10-15 kg for 30-60 minutes. The supernatant containing the refolded IFN-γ muteins was retained and the pellet was discarded.

The dialyzed supernatant was then loaded onto a SP Sepharose High Performance column (Cytiva) that was pre-equilibrated with 50 mM Tris pH 7 and 5 mM tris(2-carboxyethyl) phosphine (TCEP). After loading, the column was washed with 50 mM Tris pH 7 and 5 mM TCEP for ten column volumes. The IFN-γ mutein was eluted using a 10-40 column volume linear gradient of 50 mM Tris pH 7, 5 mM TCEP, and 1 M sodium chloride. The elution fractions were assessed for presence of IFN-γ mutein using SDS PAGE analysis and Coomassie blue staining. Fractions containing high purity IFN-γ mutein were pooled and stored in −80° C.

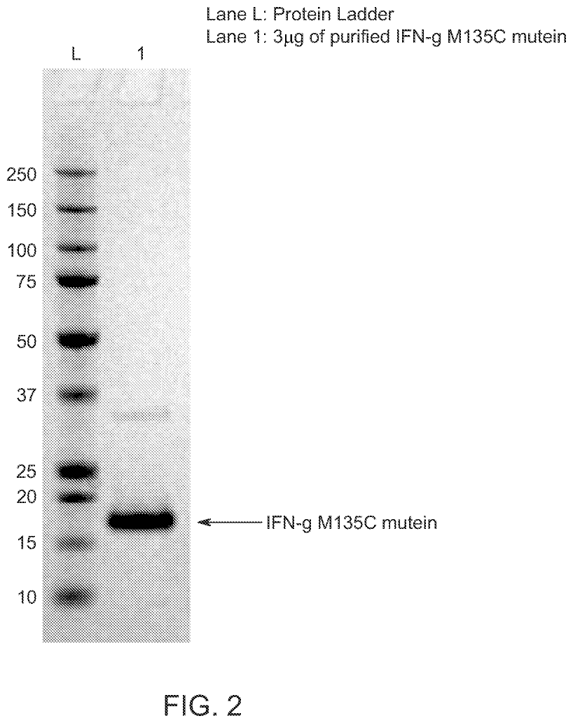

SDS PAGE analysis and Coomassie blue staining of the final purified IFN-γ-M135C mutein are shown in FIG. 2.

Overall, the M135C mutein protein yield of this process was ˜3 μg purified hIFN-γ-M135C mutein.

### Example 2

### Analysis of rhIFN-γ-1b Muteins by Liquid Chromatography-Mass Spectrometry (LC-MS)

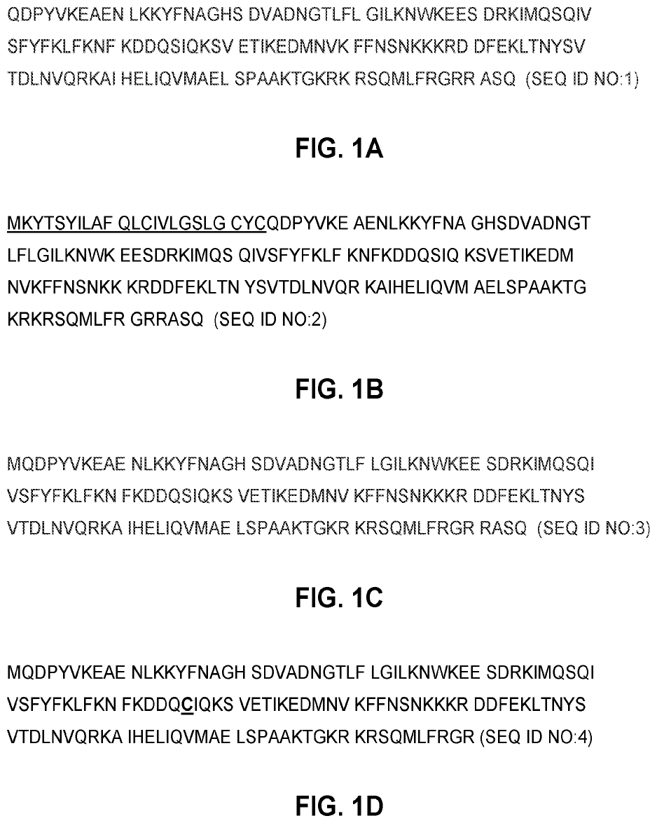

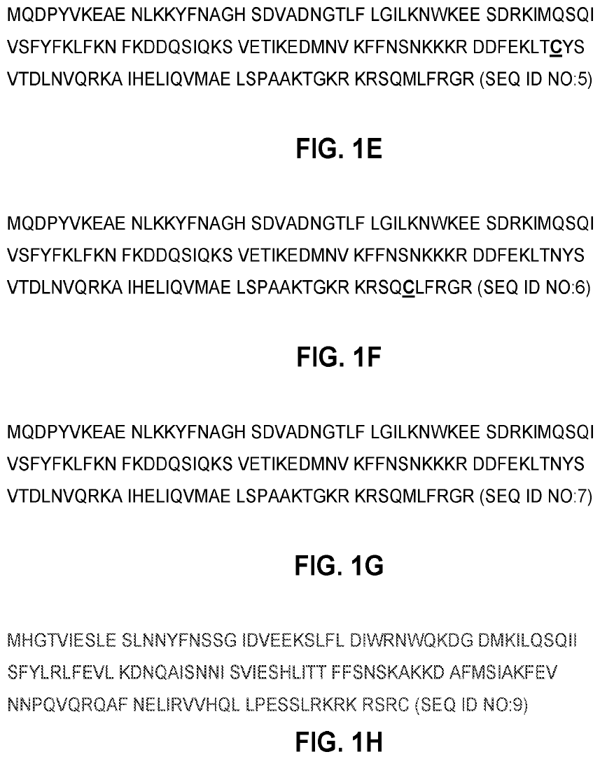

Interferon-γ-1 b cysteine muteins were prepared as described in Example 1 by mutating a single amino acid residue of the interferon-γ-1 b protein (SEQ ID NO:7). The muteins each comprised a single amino acid residue substituted with a cysteine at one of serine 66 (S66C), asparagine 98 (N98C), or methionine 135 (M135C). The sequences of the resulting muteins are shown in FIGS. 1D-1F. The IFN-γ-1 b protein had a calculated molecular weight of 16,464.9 Da. Each cysteine mutein was expected to show a specific mass shift from the IFN-γ-1 b protein due to the difference between the IFN-γ-1 b protein amino acid mass and the mass of the substituted cysteine residue. The calculated molecular weight of the IFN-γ-1b-M135C was 16,436.8 Da, the calculated molecular weight of the IFN-γ-1 b N98C was 16,453.8 Da, and the calculated molecular weight of the IFN-γ-1 b S66C was 16,480.9 Da.

To determine the mutein intact masses, Ultrahigh Performance Liquid Chromatography with Electrospray Ionization Mass Spectrometry (LC-ESI-MS) was used to generate mass spectra for deconvolution using Byos software (Protein Metrics, Inc, v3.10) to determine the observed mass of each full length mutein, with an expected error of ±1 Da from the calculated value. The deconvoluted mass spectra for the three muteins are shown in FIG. 3. The highest intensity mass peak matches with the calculated mutein mass in each case. The difference between the calculated and observed masses is shown as D in each panel. Deconvoluted mass spectra were generated for each of the cysteine IFN-γ-1b muteins, with results summarized in Table 2. The observed masses match to the calculated masses within 1 Da in all cases. The results show that the observed mutein masses are consistent with the calculated masses for the expressed full-length muteins.

TABLE 2
Calculated and observed masses for all full-length mutein proteins

| | Calculated | Observed | Delta |
|---|---|---|---|
| Mutein | MW (Da) | (Da) | Mass (Da) |
| IFN-γ-1b-M135C | 14436.8 | 14437.0 | +0.2 |
| IFN-γ-1b-N98C | 14453.8 | 14453.5 | −0.3 |
| IFN-γ-1b-S66C | 14480.9 | 14480.8 | −0.1 |

### Example 3

### Analysis of rhIFN-γ-1b Muteins by Liquid Chromatography-Mass Spectrometry (LC-MS)

In order to confirm the specific position of each cysteine substitution in the amino acid sequence of the three IFN-γ-1b muteins, the muteins were analyzed by LC-MS/MS and sequence analysis.

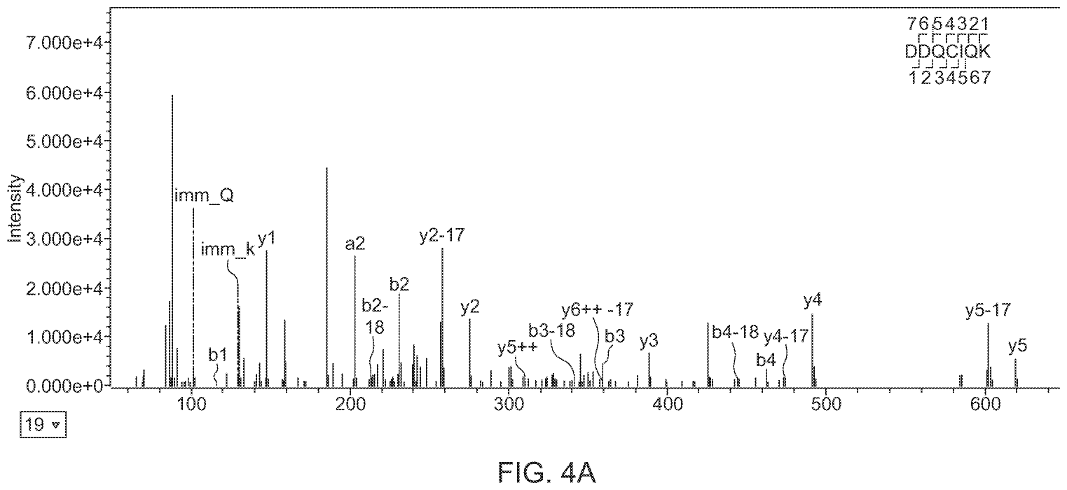

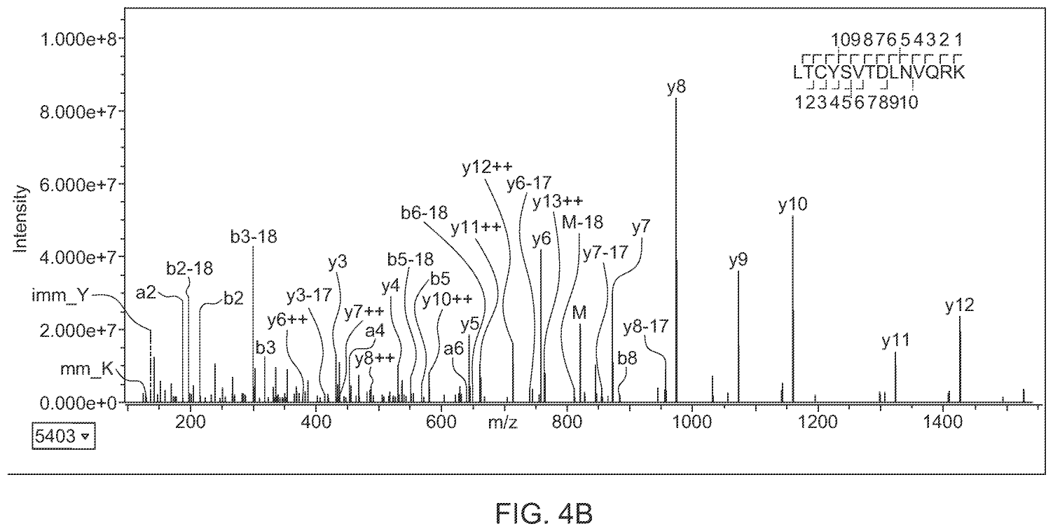

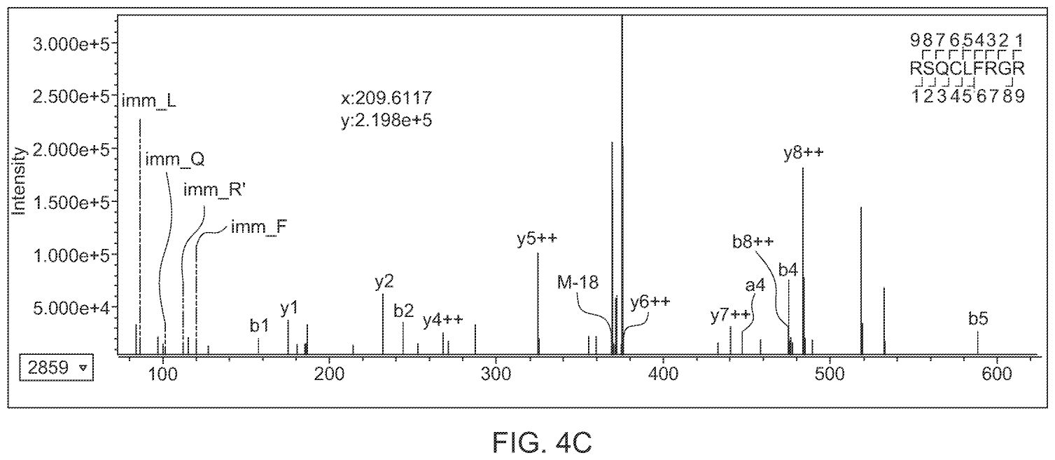

Interferon-γ-1b cysteine muteins S66C, N98C, and M135C were prepared as described in Example 1. The cysteine mutein proteins were subjected to proteolytic digestion with LysC protease. The digested muteins were analyzed by layer chromatography and then tandem mass spectroscopy (LC-MS/MS) analysis followed by database searching with the Byonic algorithm from the Byos software suite. The LysC peptide containing the targeted cysteine substitution can be identified by algorithms which match the MS/MS fragmentation spectrum of the modified peptide to a theoretical fragmentation spectrum generated by in silico digestion of the mutein amino acid sequence. LysC peptides which contain the mutated cysteine residues are listed in Table 3. Peptide fragment ion matches from a database search containing the mutein sequences using Byonic are shown for each of the muteins in FIGS. 4A-4C.

TABLE 3
LysC peptide sequences containing the introduced
cysteine for each mutein along with
corresponding wild type peptides

| | Mutein LysC | IFN-γ-1b LysC |
|---|---|---|
| Mutein | Peptide | Peptide |
| IFN-γ-M135C | RSQCLFRGR | RSQMLFRGR |
| IFN-γ-N98C | LTCYSVTDLNVQRK | LTNYSVTDLNVQRK |
| IFN-γ-S66C | DDQCIQK | DDQSIQK |

Database search results showing MS/MS spectra with matches to peptide fragment ions from cysteine containing mutein peptides highlighted are shown in FIGS. 4A-4C. Matched b-ions are shown in blue (labels beginning with “a” or “b”) and matched y-ions are shown in red (labels beginning with “y”). The inset in upper right corner of each spectrum shows the fragment ion coverage for the mutein peptide sequence.

Specific fragment ion matches to peptides generated by proteolytic digestion can be used to confirm the identity and position of a mutated amino acid residue within a peptide sequence. The peptide fragment ions matched by software analysis of the mutein digest are summarized in the peptide sequence shown in the upper right corner of each panel in FIGS. 4A-4C. The bars above the peptide sequence indicate a match to the peptide sequence that contains the residues to the right of the matched amino acid sequence (b-ions). Bars below the sequence similarly indicate a match to the amino acid sequence found to the left of the bar (y-ions). Several b- and y-ion matches for peptide fragments containing the mutated cysteine residue in the correct peptide sequence are positively identified in all three muteins, with sequence coverage sufficient to confirm the identity and position of the cysteine mutation within the amino acid sequence.

### Example 4

### Preparation of rhIFN-γ-1b-M135C PEGylated with Branched 40 kDa mPEG-Maleimide Derivative

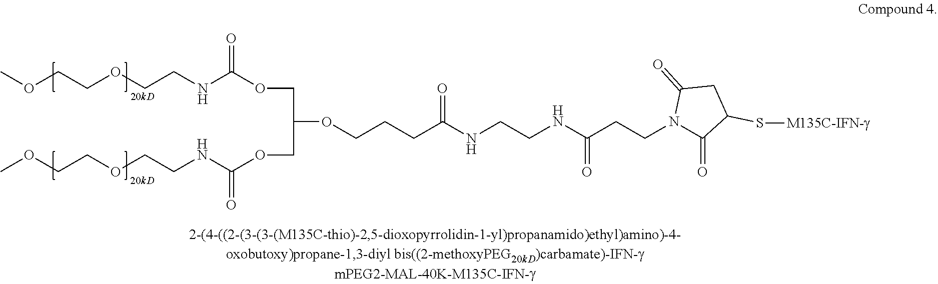

The branched mPEG-maleimide derivative reagent, mPEG2-MAL-40K,

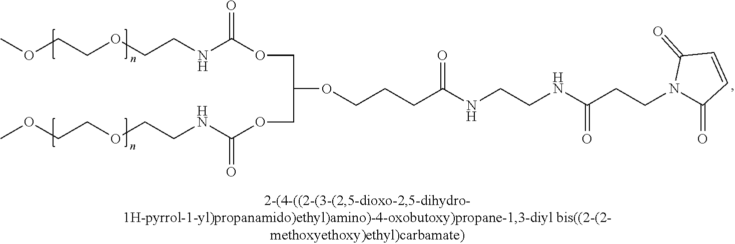

was used to prepare the subject rhIFN-γ-1b-M135C conjugates, where the weight average molecular weight of the PEG reagent used to prepare the conjugates was about 40,000 daltons.

rhIFN-γ-1 b-M135C was prepared as described in Example 1. A forty-fold excess (relative to the amount of rhIFN-γ-1 b-M135C in a measured aliquot of the stock rhIFN-γ-1 b-M135C solution) of the mPEG2-MAL-40K was dissolved in Milli-Q water to form a 10% reagent solution. The 10% reagent solution was quickly added to the aliquot of stock rhIFN-γ-1b-M135C solution (in 50 mM Tris, 500 mM NaCl, 5 mM TCEP, pH 7.0) and mixed well. The final protein concentration was adjusted to 0.7 mg/mL by addition of Milli-Q water. The reaction solution was placed on a Slow Speed Lab Rotator for overnight at room temperature to allow reaction to go to completion to provide mPEG2-MAL-40K-M135C—IFN-γ conjugates (Compound 4).

Because the PEGylation reaction was carried out around pH 7, attachment of the PEG derivative to rhIFN-γ-1 b-M135C was expected to be selective to the introduced M135C cysteine. The maleimide group of the PEG reagent can react with the sulfhydryl group of the M135C cysteine to form a thioether linkage.

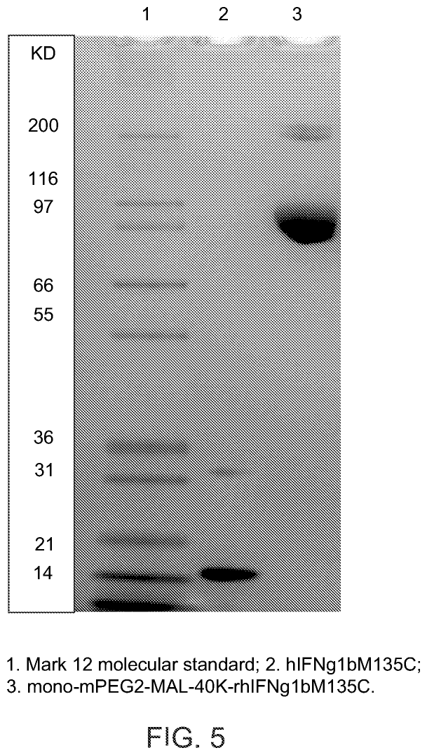

A cation-exchange chromatography method using Sepharose SP HP column along with sodium phosphate buffers was used to purify the PEGylated rhIFN-γ-1b-M135C conjugate. The purified PEGylated rhIFN-γ-1b-M135C conjugate was identified by SDS-PAGE analysis. FIG. 5 shows the SDS-PAGE gel result of a purified mono-PEGylated rhIFN-γ-1b-M135C conjugate, Compound 4. The purified conjugate was of about 95% purity and had <1% of unreacted rhIFN-γ-1b-M135C.

Following this same general approach, other IFN-γ mutein conjugates can be prepared with branched mPEG-maleimide reagents having other weight average molecular weights, e.g. 10 kDa, 30 kDa, 60 kDa, etc. as described herein.

### Example 5

### Preparation of rhIFN-γ-1b-M135C PEGylated with Branched 20 kDa mPEG-Maleimide Derivative

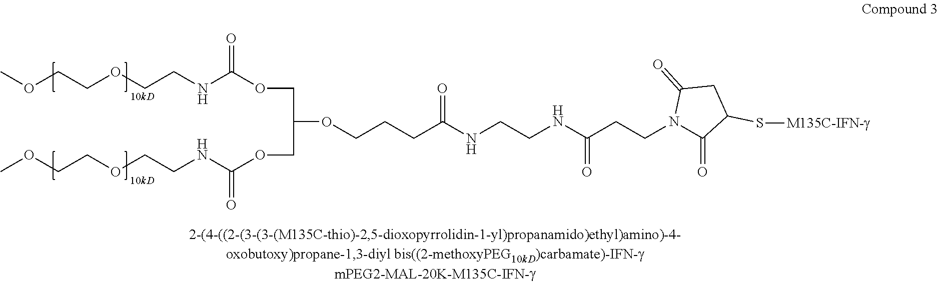

The branched mPEG-maleimide derivative PEG reagent, mPEG2-MAL-20K,

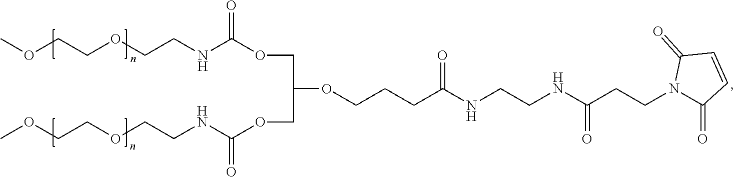

was used to prepare the subject rhIFN-γ-1b-M135C conjugates, where the weight average molecular weight of the PEG reagent used to prepare the conjugates was about 20,000 daltons.

rhIFN-γ-1 b-M135C was prepared as described in Example 1. A twenty-fold excess (relative to the amount of rhIFN-γ-1 b-M135C in a measured aliquot of the stock rhIFN-γ-1b-M135C solution) of the mPEG2-MAL-20K reagent was dissolved in Milli-Q water to form a 10% reagent solution. The 10% reagent solution was quickly added to the aliquot of stock rhIFN-γ-1 b-M135C solution (in 50 mM Tris, 500 mM NaCl, 5 mM TCEP, pH 7.0) and mixed well. The final protein concentration was adjusted to 1 mg/mL by addition of Milli-Q water. The reaction solution was placed on a Slow Speed Lab Rotator for 2 hours at room temperature to allow reaction to go to completion to provide mPEG2-MAL-20K-M135C—IFN-γ conjugates (Compound 3).

Because the PEGylation reaction was carried around pH 7, attachment of the PEG derivative to rhIFN-γ-1b-M135C is expected to be selective to the introduced M135C cysteine. The maleimide group of the PEG reagent can react with the sulfhydryl group of the M135C cysteine to form a thioether linkage.

The conjugate solution was characterized by RP-HPLC. The PEGylation reaction yielded 90% mono-conjugate (one PEG attached to rhIFN-γ-1b-M135C).

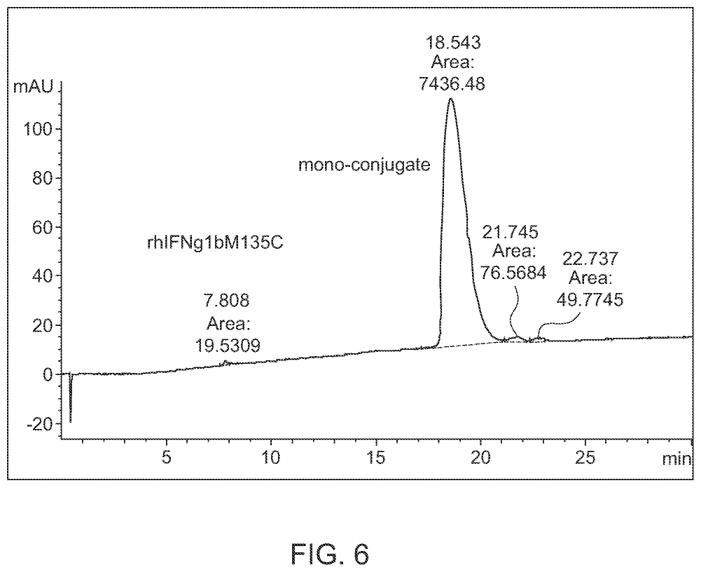

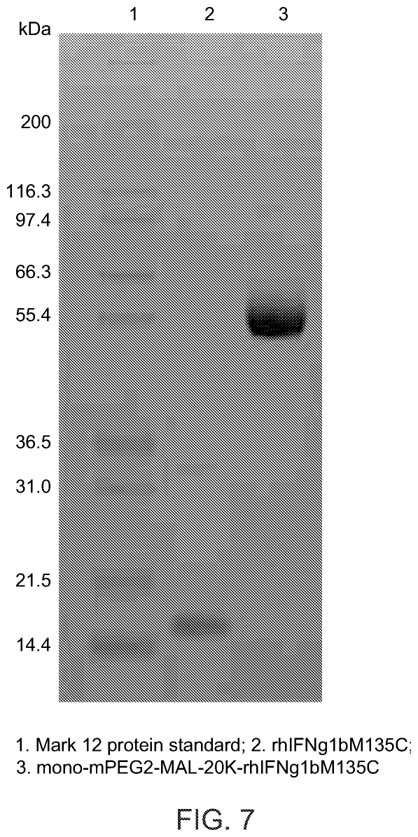

A cation-exchange chromatography method using HiTrap CaptoSP ImpRes column along with sodium phosphate buffers was used to purify the PEGylated rhIFN-γ-1 b-M135C conjugate. The purified PEGylated rhIFN-γ-1 b-M135C conjugate was identified by RP-HPLC and SDS-PAGE analysis. FIG. 6 shows the RP-HPLC result of a purified mono-PEGylated rhIFN-γ-1 b-M135C conjugate (mono-conjugate). The purified conjugate was of 98% purity and had <1% of unreacted rhIFN-γ-1 b-M135C. FIG. 7 shows the SDS-PAGE gel result of a purified mono-PEGylated rhIFN-γ-1 b-M135C conjugate. The gel showed similar result to the RP-HPLC result.

### Example 6

### Preparation of rhIFN-γ-1b-M135C PEGylated with Linear 20 kDa mPEG-Maleimide Derivative

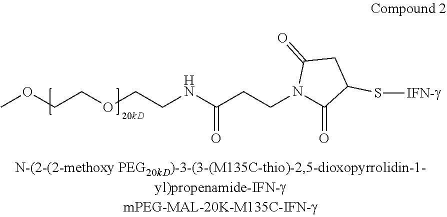

The linear mPEG-maleimide derivative PEG reagent, mPEG-MAL-20K,

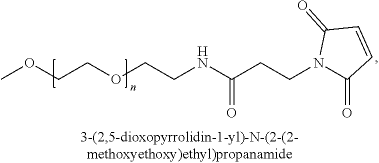

was used to prepare the subject rhIFN-γ-1b-M135C conjugates, where the weight average molecular weight of the PEG reagent used to prepare the conjugates was about 20,000 daltons.

rhIFN-γ-1 b-M135C was prepared as described in Example 1. A twenty-fold excess (relative to the amount of rhIFN-γ-1 b-M135C in a measured aliquot of the stock rhIFN-γ-1b-M135C solution) of the mPEG-MAL-20K reagent was dissolved in Milli-Q water to form a 10% reagent solution. The 10% reagent solution was quickly added to the aliquot of stock rhIFN-γ-1 b-M135C solution (in 50 mM Tris, 500 mM NaCl, 5 mM TCEP, pH 7.0) and mixed well. The final protein concentration was adjusted to 1 mg/mL by addition of Milli-Q water. The reaction solution was placed on a Slow Speed Lab Rotator for 2 hrs at room temperature to allow reaction to go to completion to provide mPEG-MAL-20K-M135C—IFN-γ conjugates (Compound 2).

Because the PEGylation reaction was carried around pH 7, attachment of the PEG derivative to rhIFN-γ-1b-M135C was expected to be selective to the introduced M135C cysteine. The maleimide group of the PEG reagent can react with the sulfhydryl group of the M135C cysteine to form a thioether linkage.

The conjugate solution was characterized by RP-HPLC. The PEGylation reaction yielded 87% mono-conjugate (one PEG attached to rhIFN-γ-1b-M135C).

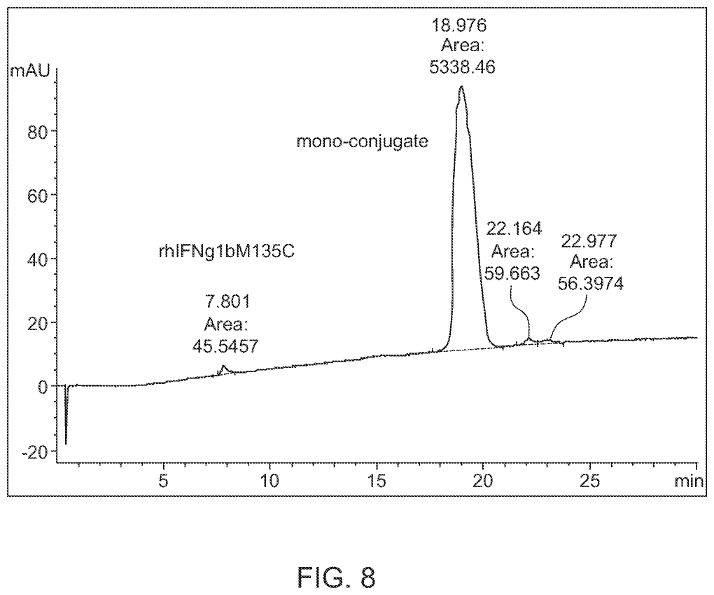

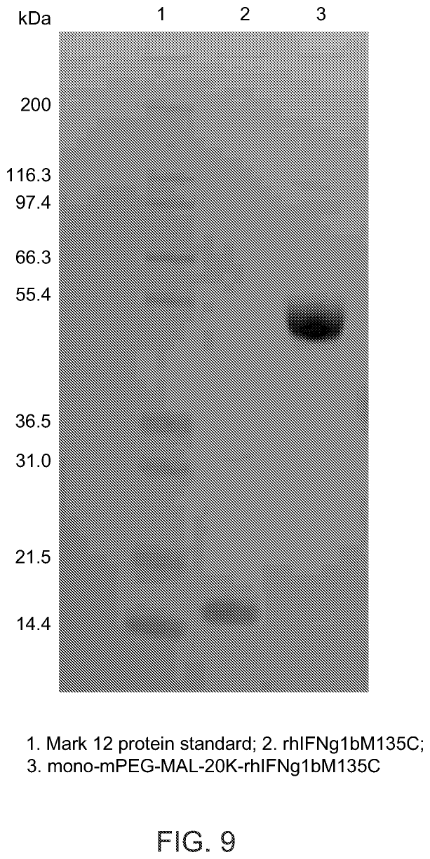

A cation-exchange chromatography method using HiTrap CaptoSP ImpRes column along with sodium phosphate buffers was used to purify the PEGylated rhIFN-γ-1 b-M135C conjugate. The purified PEGylated rhIFN-γ-1 b-M135C conjugate was identified by RP-HPLC and SDS-PAGE analysis. FIG. 8 shows the RP-HPLC result of a purified mono-PEGylated rhIFN-γ-1 b-M135C conjugate (mono-conjugate). The purified conjugate was of 97% purity and had <1% of unreacted rhIFN-γ-1 b-M135C. FIG. 9 shows the SDS-PAGE gel result of a purified mono-PEGylated rhIFN-γ-1 b-M135C conjugate. The gel showed similar result to the RP-HPLC result.

Using this same approach, other IFN-γ mutein conjugates can be prepared with linear mPEG-maleimide reagents having other weight average molecular weights, e.g. 30 kDa, 40 kDa, 50 kDa, 60 kDa, etc. as described herein.

### Example 7

### Preparation of rhIFN-γ-1b-M135C PEGylated with Linear 10 kDa mPEG-Maleimide Derivative

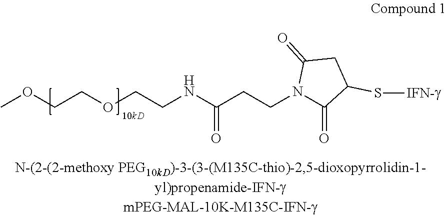

The linear mPEG-maleimide derivative PEG reagent, mPEG-MAL-10K,

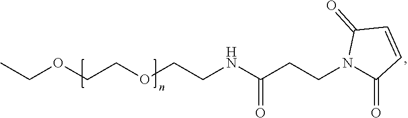

was used to prepare the subject rhIFN-γ-1 b-M135C conjugates, where the weight average molecular weight of the PEG reagent used to prepare the conjugates was about 10,000 daltons.

rhIFN-γ-1b-M135C was prepared as described in Example 1. A 10% mPEG-MAL-10K stock solution was prepared in Milli-Q water and a ten-fold excess of this PEG reagent was quickly added to an aliquot of stock rhIFN-γ-1 b-M135C solution (in 50 mM Tris, 500 mM NaCl, 5 mM TCEP, pH 7.0). The final protein concentration was adjusted to 1 mg/mL by addition of Milli-Q water. The reaction was immediately set on a tube revolver (Slow Speed Lab Rotator) for two hours at room temperature to allow to reaction to go to completion to provide mPEG-MAL-10K-M135C—IFN-γ conjugates (Compound 1), and subsequently analyzed by HPLC and purified by cation-exchange chromatography.

Because the PEGylation reaction was carried around pH 7, attachment of the PEG derivative to rhIFN-γ-1b-M135C was expected to be selective to the introduced M135C cysteine. The maleimide group of the PEG reagent can react with the sulfhydryl group of the M135C cysteine to form a thioether linkage.

The conjugate solution was characterized by RP-HPLC. The PEGylation reaction yielded 90% mono-conjugate (one PEG attached to rhIFN-γ-1b-M135C) and 10% unreacted rhIFN-γ-M135C.

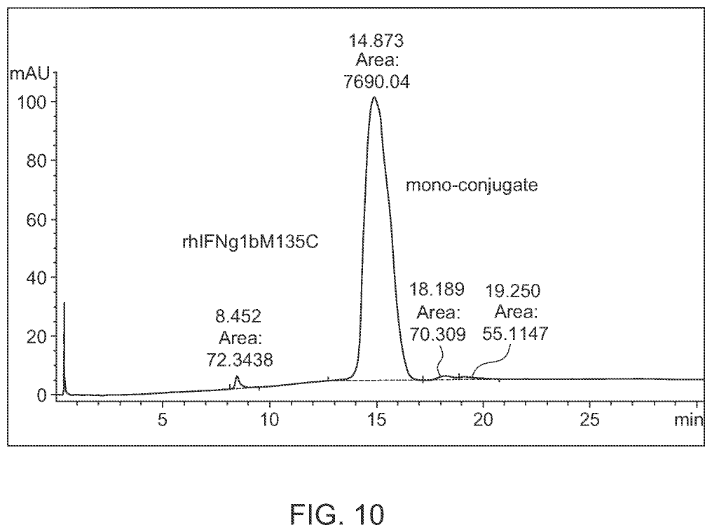

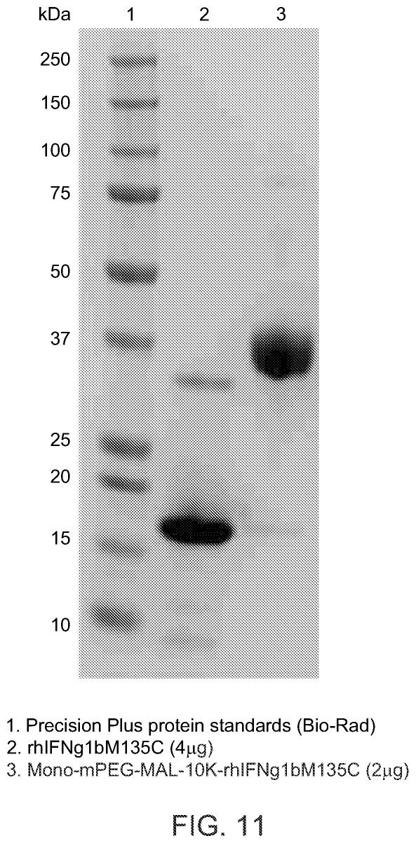

A cation-exchange chromatography method using HiTrap CaptoSP ImpRes column along with sodium phosphate buffers was used to purify the PEGylated rhIFN-γ-1 b-M135C conjugate. The purified PEGylated rhIFN-γ-1 b-M135C conjugate was identified by RP-HPLC and SDS-PAGE analysis. FIG. 10 shows the RP-HPLC result of a purified mono-PEGylated rhIFN-γ-1 b-M135C conjugate. The test conjugate was of 97.5% purity and contained <1% of unreacted rhIFN-γ-1 b-M135C. FIG. 11 shows the SDS-PAGE gel result of a purified mono-PEGylated rhIFN-γ-1b-M135C conjugate. The gel showed similar result to the RP-HPLC result.

### Example 8

### Preparation of rhIFN-γ-1b-N98C PEGylated with Branched 20 kDa mPEG-Maleimide Derivative

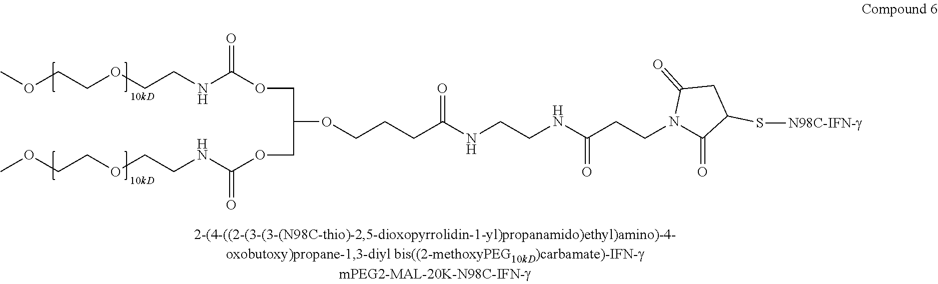

The branched mPEG-maleimide derivative PEG reagent, mPEG2-MAL-20K,

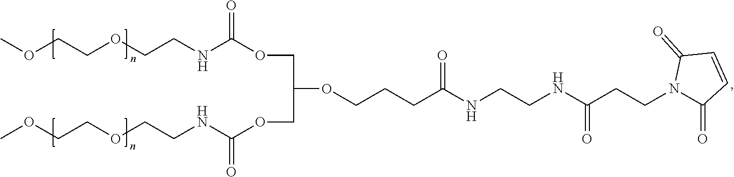

was used to prepare the subject rhIFN-γ-1b-N98C conjugates, where the weight average molecular weight of the PEG reagent used to prepare the conjugates was about 20,000 daltons.

rhIFN-γ-1 b-N98C was prepared as described in Example 1. A 20% mPEG2-MAL-20K stock solution was prepared in Milli-Q water and a sixty-fold excess of this PEG reagent was quickly added to an aliquot of stock rhIFN-γ-1 b-N98C solution (in 50 mM Tris, 500 mM NaCl, 5 mM TCEP, pH 7.0). The final protein concentration was adjusted to 1 mg/mL by addition of Milli-Q water. The reaction was immediately set on a tube revolver (Slow Speed Lab Rotator) for two hours at room temperature to provide mPEG2-MAL-20K-N98C—IFN-γ conjugates (Compound 6), and subsequently analyzed by RP-HPLC and purified by cation-exchange chromatography.

Because the PEGylation reaction was carried around pH 7, attachment of the PEG derivative to rhIFN-γ-1 b-N98C was expected to be selective to the introduced N98C cysteine. The maleimide group of the PEG reagent can react with the sulfhydryl group of the N98C cysteine to form a thioether linkage.

The conjugate solution was characterized by RP-HPLC. The PEGylation reaction yielded 99% mono-conjugate (one PEG attached to rhIFN-γ-1 b-N98C) and 1% unreacted rhIFN-γ-1 b-N98C.

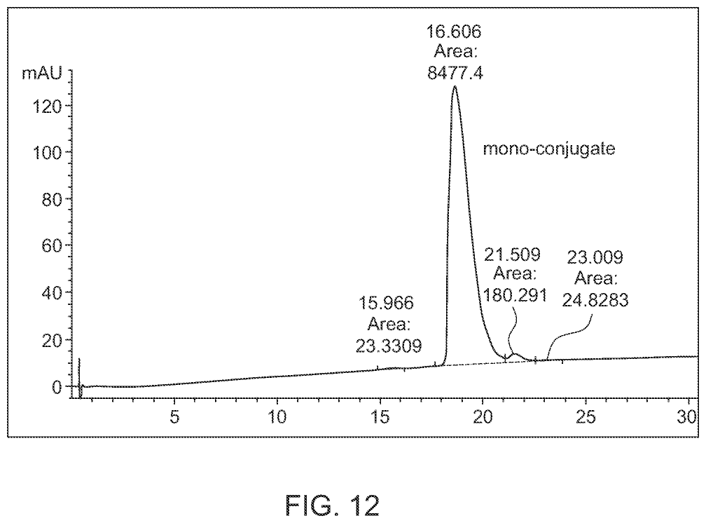

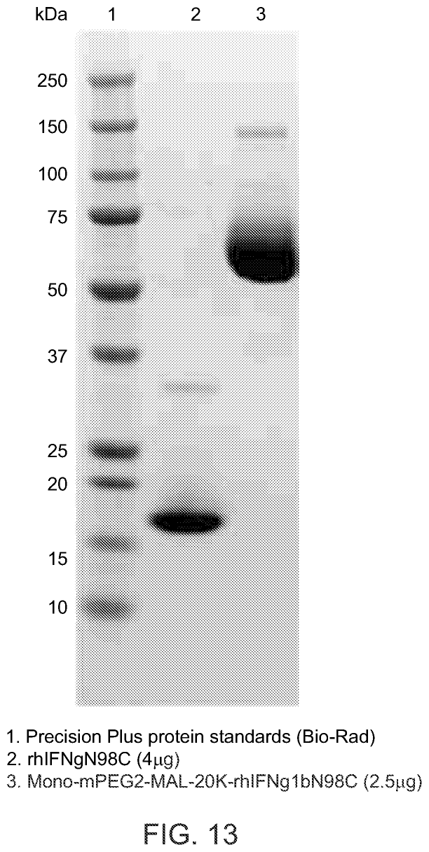

A cation-exchange chromatography method using HiTrap CaptoSP ImpRes column along with sodium phosphate buffers was used to purify the PEGylated rhIFN-γ-1 b-N98C conjugate. The purified PEGylated rhIFN-γ-1 b-N98C conjugate was identified by RP-HPLC and SDS-PAGE analysis. FIG. 12 shows the RP-HPLC result of a purified mono-PEGylated rhIFN-γ-1 b-N98C conjugate. The test conjugate was of 97% purity. FIG. 13 shows the SDS-PAGE gel result of a purified mono-PEGylated rhIFN-γ-1 b-N98C conjugate. The gel showed similar result to the RP-HPLC result.

Following this same general approach, other rhIFN-γ-1 b-N98C mutein conjugates can be prepared with linear and branched mPEG-maleimide reagents having various weight average molecular weights, e.g. 10 kDa, 20 kDa, 30 kDa, 40 kDa, 50 kDa, and 60 kDa.

### Example 9

### Preparation of rhIFN-γ-1b-S66C PEGylated with Branched 20 kDa mPEG-Maleimide Derivative

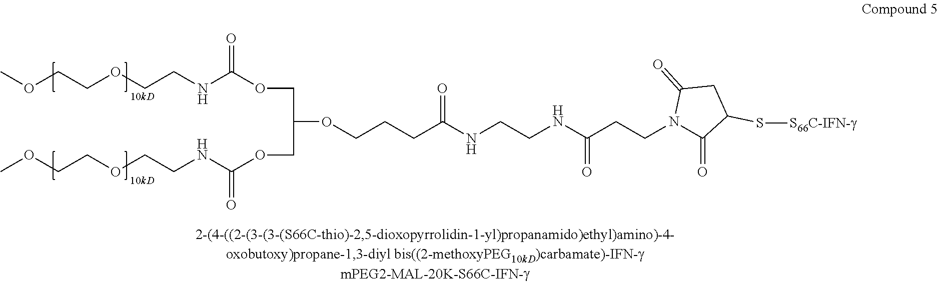

The branched mPEG-maleimide derivative PEG reagent, mPEG2-MAL-20K,

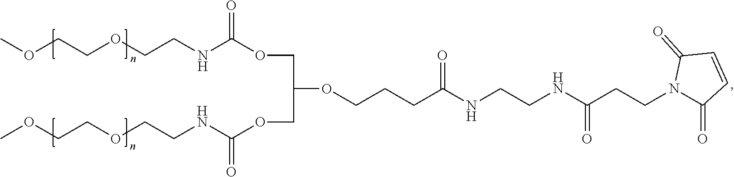

was used to prepare the subject rhIFN-γ-1b-S66C conjugates, where the weight average molecular weight of the PEG reagent used to prepare the conjugates was about 20,000 daltons.

rhIFN-γ-1 b-S66C was prepared as described in Example 1. A twenty-fold excess (relative to the amount of rhIFN-γ-1b-S66C in a measured aliquot of the stock rhIFN-γ-1 b-S66C solution) of the mPEG2-MAL-20K was dissolved in Milli-Q water to form a 20% reagent solution. The 20% reagent solution was quickly added to the aliquot of stock rhIFN-γ-1 b-S66C solution (in 50 mM Tris, 500 mM NaCl, 5 mM TCEP, pH 7.0) and mixed well. The final protein concentration was adjusted to 1 mg/mL by addition of Milli-Q water. The reaction solution was placed on a Slow Speed Lab Rotator for 2 hrs at room temperature to allow reaction to go to completion to provide mPEG2-MAL-20K-S66C—IFN-γ conjugates (Compound 5).

Because the PEGylation reaction was carried around pH 7, attachment of the PEG derivative to rhIFN-γ-1 b-S66C was expected to be selective to the introduced S66C cysteine. The maleimide group of the PEG reagent can react with the sulfhydryl group of the S66C cysteine to form a thioether linkage.

The conjugate solution was characterized by RP-HPLC. The PEGylation reaction yielded about 87% mono-conjugate (one PEG attached to rhIFN-γ-1 b-S66C).

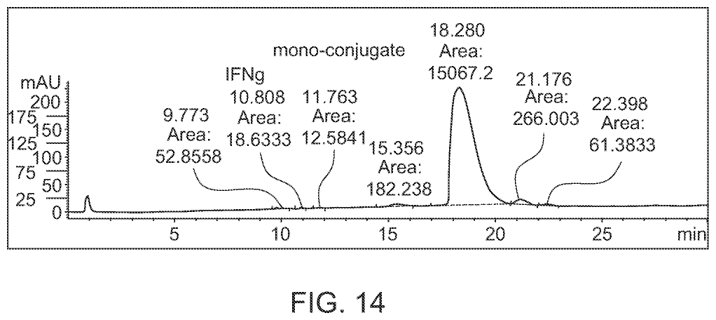

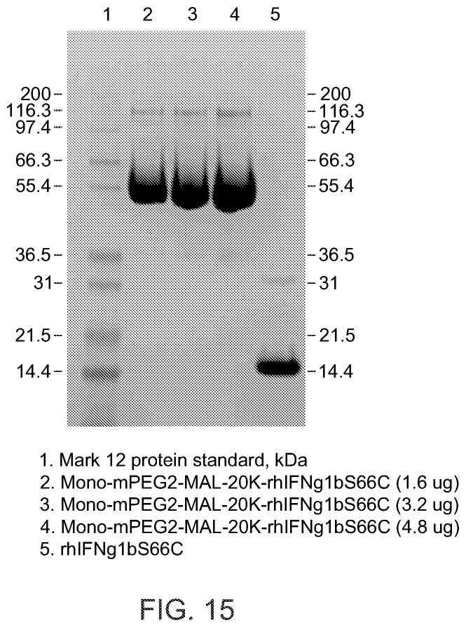

A cation-exchange chromatography method using HiTrap CaptoSP ImpRes column along with sodium phosphate buffers was used to purify the PEGylated rhIFN-γ-1 b-S66C conjugate. The purified PEGylated rhIFN-γ-1 b-S66C conjugate was identified by RP-HPLC and SDS-PAGE analysis. FIG. 14 shows the RP-HPLC result of a purified mono-PEGylated rhIFN-γ-1 b-S66C conjugate. The purified conjugate was of 96% purity and had <1% of unreacted rhIFN-γ-1 b-S66C. FIG. 15 shows the SDS-PAGE gel result of a purified mono-PEGylated rhIFN-γ-1b-S66C conjugate. The gel showed similar result to the RP-HPLC result.

Following this same general approach, other rhIFN-γ-1 b-S66C mutein conjugates can be prepared with linear and branched mPEG-maleimide reagents having various weight average molecular weights, e.g. 10 kDa, 20 kDa, 30 kDa, 40 kDa, 50 kDa, and 60 kDa.

### Example 10

### Preparation of Ring-Opened rhIFN-γ-1b-M135C PEGylated with Branched 20 kDa mPEG-Maleimide Derivative

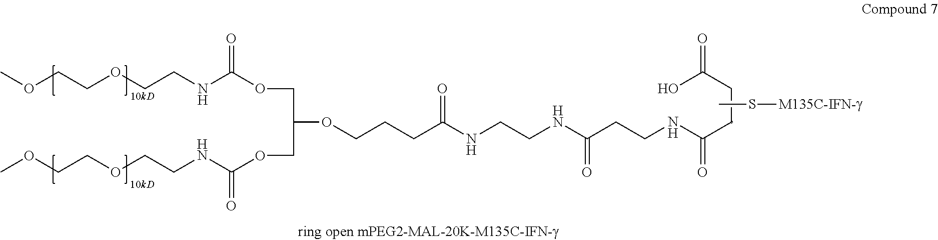

The branched mPEG-maleimide derivative PEG reagent, mPEG2-MAL-20K,

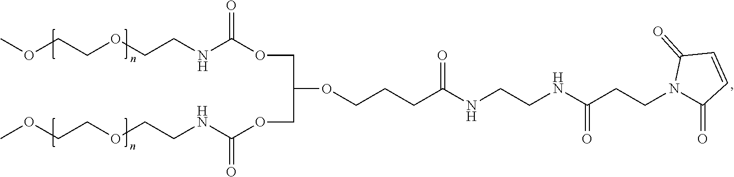

was used to prepare rhIFN-γ-1b-M135C conjugates, where the weight average molecular weight of the PEG reagent used to prepare the conjugates was about 20,000 daltons. rhIFN-γ-1b-M135C was prepared as described in Example 1.

A 10% branched mPEG2-MAL-20K stock solution was prepared in Milli-Q water and a ten-fold excess of the PEG reagent was quickly added to an aliquot of stock rhIFN-γ-1b-M135C solution (in 50 mM Tris, 500 mM NaCl, 5 mM TCEP, pH 7.0). The final protein concentration was adjusted to 1 mg/mL by addition of Milli-Q water. The reaction was immediately set on a tube revolver (Slow Speed Lab Rotator) for one hour at room temperature, and subsequently analyzed by HPLC and purified by cation-exchange chromatography.

The conjugate solution was characterized by RP-HPLC. The PEGylation reaction yielded 92.4% mono-conjugate (one PEG attached to rhIFN-γ-1b-M135C) and 7.6% unreacted rhIFN-γ-M135C.

A cation-exchange chromatography method using HiTrap CaptoSP ImpRes column along with sodium phosphate buffers was used to purify the PEGylated rhIFN-γ-1b-M135C conjugate under acidic conditions. The purified PEGylated rhIFN-γ-1b-M135C conjugate was subsequently treated with base to force opening of the succinimide ring. The pH of the purified conjugate was adjusted to pH 8.5, and then incubated at room temperature for 24 hours. At the end of the incubation period, the pH was lowered to 7.4 and the final conjugate was buffer exchanged and stored in the formulation buffer.

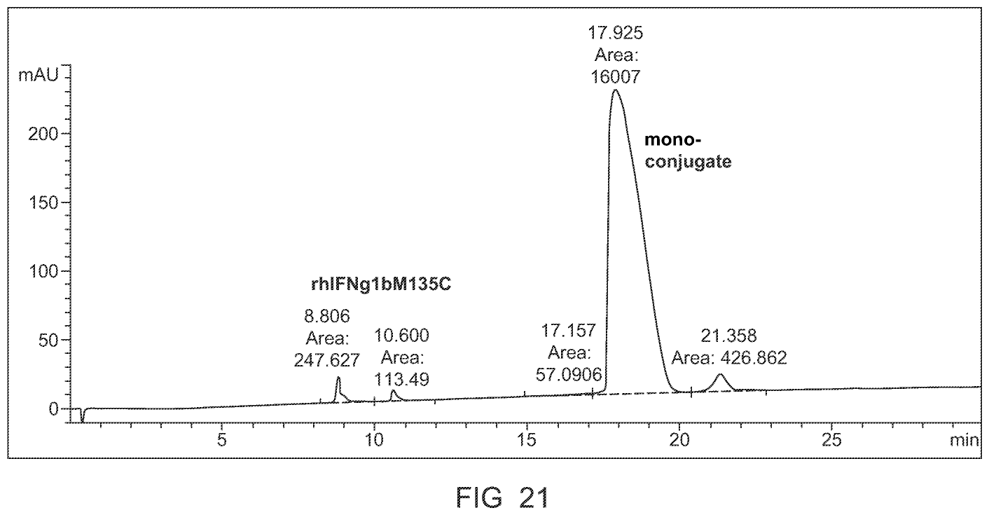

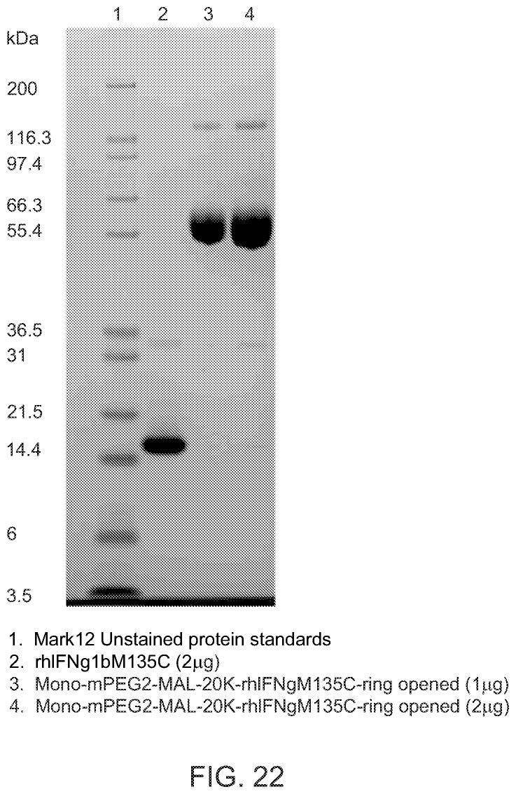

The ring opened PEGylated rhIFN-γ-1b-M135C conjugate (Compound 7) was identified by RP-HPLC and SDS-PAGE analysis. FIG. 21 shows the RP-HPLC results for the ring opened mono-PEGylated rhIFN-γ-1b-M135C conjugate. The test conjugate had 95.0% purity and contained 2.1% of unreacted rhIFN-γ-1b-M135C. FIG. 22 shows the SDS-PAGE gel result of a ring opened mono-PEGylated rhIFN-γ-1b-M135C conjugate. The gel showed similar result to the RP-HPLC result.

Using this same approach, other conjugates can be prepared with linear and branched mPEG-maleimide reagents having other weight average molecular weights.

### Example 11

### Purification of Ring-Opened rhIFN-γ-1b-M135C PEGylated with Branched 20 kDa mPEG-Maleimide Derivative

A ring-opened mono-PEGylated rhIFN-γ-1b-M135C conjugate was prepared as described in Example 10. The resulting composition was further purified to remove a majority of the unreacted rhIFN-γ-1b-M135C protein. A cation-exchange chromatography method using HiTrap CaptoSP ImpRes column along with sodium phosphate buffers was used to purify the ring opened PEGylated rhIFN-γ-1b-M135C conjugate under acidic conditions.

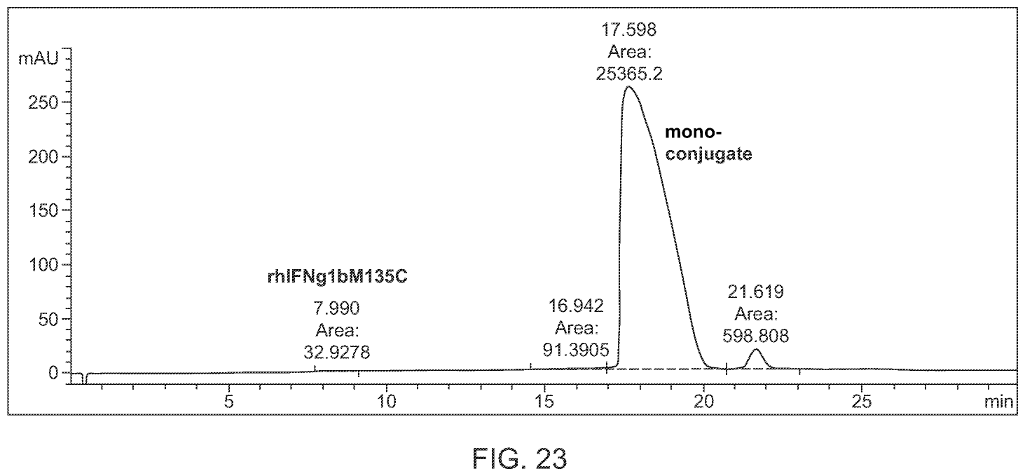

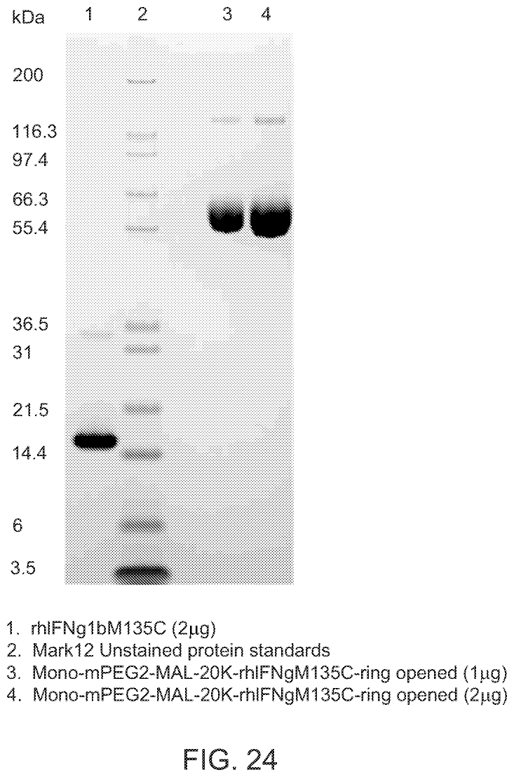

The purified ring opened PEGylated rhIFN-γ-1b-M135C conjugate was identified by RP-HPLC and SDS-PAGE analysis. FIG. 23 shows the RP-HPLC for the purified ring opened mono-PEGylated rhIFN-γ-1b-M135C conjugate. The test conjugate had 97.2% purity and contained <0.2% of unreacted rhIFN-γ-1 b-M135C. FIG. 24 shows the SDS-PAGE gel result of the purified ring opened mono-PEGylated rhIFN-γ-1 b-M135C conjugate. The gel showed similar result to the RP-HPLC result.

### Example 12

### Pegylation Site Mapping of Ring Open mPEG2-MAL-20K-M135C—IFN-γ

Compound 7 was prepared as described in Example 10 above. Briefly, PEGylation of IFN-γ-1b-M135C occurs by thioether bond formation when mPEG2-MAL-20K (structure shown in Example 10) reacts with the free thiol group of Cys135 of the IFN-γ mutein. PEG site attachment at Cys135 was confirmed by LC-MS/MS using In-Source Fragmentation (LC-ISF-MS/MS), which identified PEG sites by locating a specific maleimide remnant (C₄H₂O₃; 98.004 Da) which remains attached to the cysteine thiol side chain after In-Source Fragmentation (ISF), serving as a tag to mark the specific location(s) of the original PEG-MAL-IFN-γ linkage. To identify the specific residue of PEG-MAL linkage for the compound, LysC was used to digest the conjugated IFN-γ M135C protein into peptides, which were then analyzed by LC-ISF-MS/MS and searched using Byonic Protein ID software (ProteinMetrics Inc, Version 4.3), specifying the MAL ring ISF fragmentation product C₄H₂O₃ (monoisotopic mass=98.0004 Da) as a variable modification on cysteine residues in accord with the following:

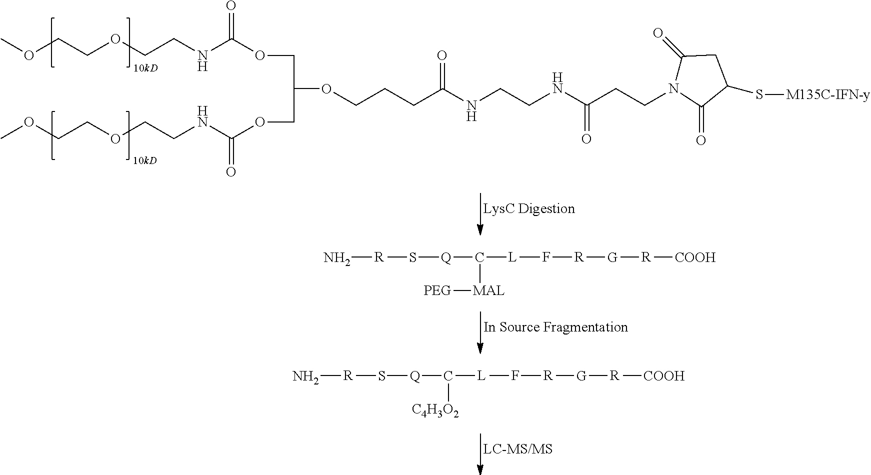

| # | b calc. | b obs. | b delta. | b++ calc | b++ obs. | Seq. | y calc. | y obs. | y++ calc. | y++ obs. | # |
|---|---|---|---|---|---|---|---|---|---|---|---|
| 1 | 157.11 | 157.109 | 0.0005 | 79.058 | | R | | | | | 9 |
| 2 | 244.14 | 244.14 | −7E−04 | 122.57 | | S | 1064.5 | | 532.8 | 532.75 | 8 |
| 3 | 372.2 | 372.197 | −0.002 | 186.6 | | Q | 977.46 | | 489.2 | 489.236 | 7 |
| 4 | 573.21 | 573.207 | −0.001 | 287.11 | | C | 849.4 | | 425.2 | 425.208 | 6 |
| 5 | 686.29 | 686.29 | −0.003 | 343.65 | | L | 648.39 | | 324.7 | 324.701 | 5 |
| 6 | 833.36 | 833.362 | 0.0008 | 417.18 | | F | 535.31 | | 268.2 | 268.158 | 4 |
| 7 | 989.46 | | | 495.23 | 495.238 | R | 388.24 | 388.236 | 194.6 | | 3 |
| 8 | 1046.5 | | | 523.75 | | G | 232.14 | 232.14 | 116.6 | | 2 |
| 9 | | | | | | R | 175.12 | 175.119 | 88.06 | | 1 |

b obs., b++ obs., y obs., and y++ obs. show the peptide fragments identified confirming C₄H₂O₃ (98.0004 Da) modification of Cys135 in IFN-γ-1b M135C.

LC-MS/MS with ISF was used to confirm the location of PEG site attachment to IFN-γ M135C at the expected Cys135 residue. The C₄H₂O₃ MAL remnant was found only on IFN-γ M135C LysC peptides 132-140 and 130-140, which both contained Cys135. Fragmentation spectra specifically assigned the attachment site to the cysteine residue, in which specific b- and y-fragments ions showed the expected mass shift of +98.0004 Da to the cysteine thiol side chain.

### Example 13

### Separation of Ring Closed and Ring Open mPEG2-MAL-20K-M135C—IFN-γ by Strong Cation Exchange (SCX) Chromatography

Incubation of the ring-closed (RC) Compound 3 at pH 8.5 was used to generate the MAL ring open (RO) form of the conjugate, Compound 7. The ring opening reaction added an additional negative charge to the conjugate at physiological pH which was used to separate and quantify the RC and RO forms of mPEG2-MAL-20K-M135C—IFN-γ by charge-based separation using strong cation exchange (SCX) chromatography. The parameters used for the separation are shown in Table 4 and include the simultaneous use of both salt and pH to achieve the separation.

TABLE 4
Strong Cation Exchange Method Conditions

| Parameter | Setting |
|---|---|
| Column | Agilent NP (Non-Porous) SP (Sulfopropyl) Column |
| | (4.6 mm × 50 mm, 1.7 μm) |
| Mobile phase A | 20 mM Na Phosphate, pH 6 |
| Mobile phase B | 20 mM Na Phosphate, pH 9 + 1M NaCl |
| Flow rate | 0.5 mL/min |
| Column Temp | 25° C. |
| Gradient: | 0-50% B in 30 minutes |
| Sample Load | 10 μg |

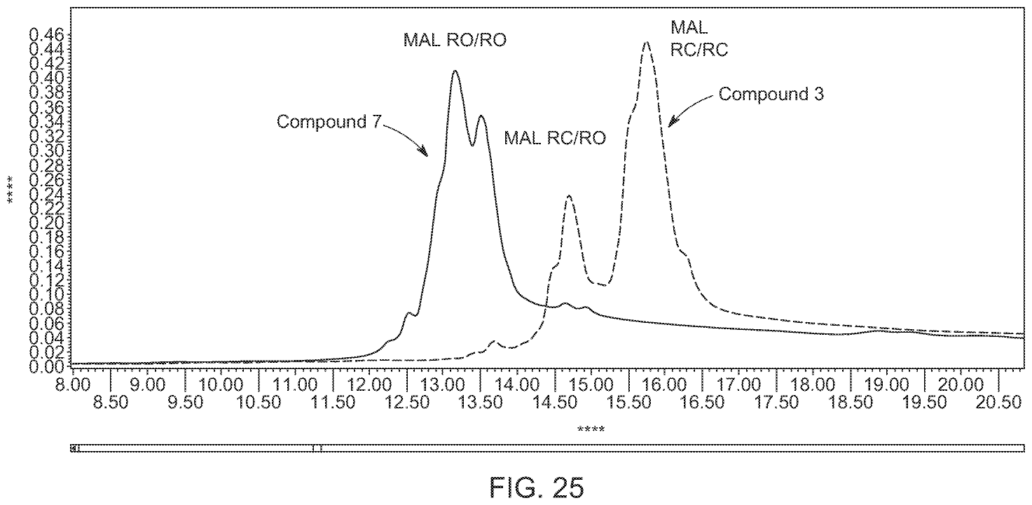

Representative SCX traces from separations of MAL-RC and MAL-RO conjugates are shown overlaid in FIG. 25.

The SCX profiles of Compound 3 and Compound 7 samples (FIG. 25) show three peak groups, one in the Compound 7 RO sample and two in the Compound 3 RC sample. Each conjugate dimer molecule has two MAL rings, one for each attached PEG group, which can be in either RC or RO form. This creates three potential MAL ring combinations, 1) RO/RO, 2) RC/RO, and 3) RC/RC, each with different retention times (RT) based upon their net charge. SCX analysis of a sample of Compound 3 showed predominantly MAL-RC/RC (RT=15.5-16.5 min), but also contained some RC/RO (14.5-15 min), and a small amount of RO/RO (13.25-14 min). The Compound 7 sample was almost completely converted to RO/RO (RT=12.5-14.0 min), with a small amount of heterodimeric RO/RC remaining (RT=14.5-15 min). The MAL-ring forms for the individual peaks were confirmed by Mass Spectrometry as described in Example 14 below.

In addition, each of the three peak groups had a partially resolved, earlier eluting second peak. Without being limited as to theory, this peak splitting is believed to be a result of deamidation at Asn26, which creates an additional negative charge on the protein, and occurred in all MAL forms of the conjugate.

### Example 14

### Determination of Intact Protein Molecular Weight of Maleimide Ring Closed and Ring Open Forms of mPEG2-MAL-20K-M135C—IFN-γ by Liquid Chromatography-Mass Spectrometry (LC-MS)

To confirm the MAL-ring form of conjugates, a modified high-resolution LC-MS technique was developed to allow discrimination between RO and RC forms of mPEG2-MAL-20K-M135C—IFN-γ MAL (Table 5). The method confirmed the MAL ring status of both Compound 3 (RC/RC) and Compound 7 (RO/RO) samples, as well as the identities of the compounds represented by the individual peaks in the SCX method separation (including RC/RO heterodimer) as described in Example 13.

TABLE 5
LC-MS Intact Mass Parameters for
Post Column Base Addition Method

| Parameter | Setting |
|---|---|
| Column | Waters Acquity C4 Protein BEH C4 |
| | (2.1 mm × 150 mm, 1.7 μm; 300 Å) |
| Mobile phase A | 0.1% Formic Acid in Water |
| Mobile phase B | 0.1% Formic Acid in Acetonitrile |
| Flow rate | 0.3 mL/min |
| Column Temp | 60° C. |
| Gradient | 15-65% B in 10 minutes |
| Sample Load | 1 μg |
| Post Column Base Addition: | 1% Diethylmethylamine in 50% |
| | Acetonitrile |
| Mass Analysis Range | 400-14,000 |
| MS Scan Time | 1 second |

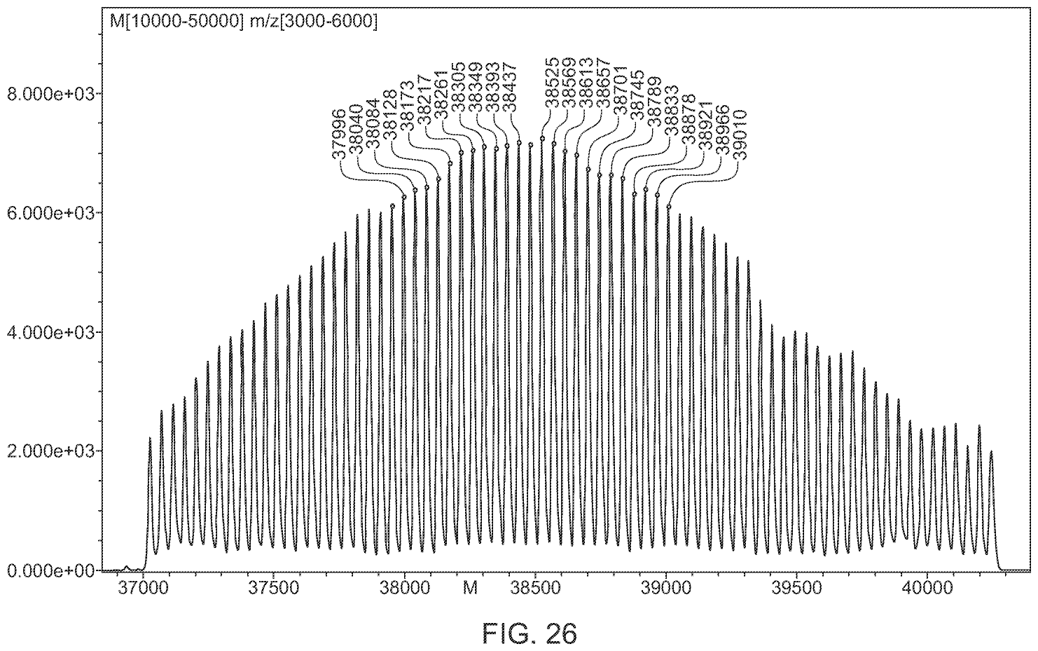

The high resolution intact mass LC-MS method relied on post-column base addition (1% diethylmethylamine in 1:1 Water/Acetonitrile) to strip charge from the conjugate molecules, which are too highly charged for MS analysis due to the presence of PEG. Base addition occurred after LC separation, but before MS analysis. The charge stripping technique, described in Table 5, shifted the charge envelope from low to high m/z values, generally >3000 m/z, which can be deconvoluted to the zero-charge state intact mass using the Byos intact mass software algorithm (ProteinMetrics, Inc; Version 4.3). A representative spectrum of deconvoluted Compound 7 is shown in FIG. 26.

The identity of the MAL ring forms separated by SCX chromatography was confirmed by intact mass analysis using LC-MS. Since LC-MS is a denaturing technique, the rhIFN-γ-M135C homodimer protein dissociates during the analysis, generating two mono-PEGylated IFN-γ protein monomers with identical masses of approximately 38.5 kD. The observed intact masses consisted of a polydisperse series of peaks separated by 44.02 Da increments that span the mass region from 37-40.5 kD, with the apex centered at approximately 38.5 kD as expected. The +44 Da mass series showed the Gaussian distribution of the individual oxyethylene PEG monomers in the branched 20 kD PEG polymer. Confirmation of MAL ring status was be obtained by comparison of the Compound 3 RC/RC polymer mass series values to the corresponding Compound 7 RO/RO mass series values. The Compound 7+44 Da mass series showed a +18 Da shift relative to the Compound 3 conjugate in all corresponding polymer peaks due to the water molecule addition that occurred during MAL ring opening, which confirmed the MAL ring opening of Compound 7 (Table 6).

TABLE 6
Comparison of +44 Da Mass Series' Observed
from Compound 3 and Compound 7

| Compound 3 | Compound 7 | Delta Mass |
|---|---|---|
| MAL Ring Closed | MAL Ring Open | (Column 2 − |
| (Mass) | (Mass) | Column 1) |
| 38,111 | 38,129 | +18 |
| 38,155 | 38,173 | +18 |
| 38,199 | 38,217 | +18 |
| 38,243 | 38,261 | +18 |
| 38,287 | 38,305 | +18 |
| 38,331 | 38,349 | +18 |
| 38,375 | 38,393 | +18 |
| 38,419 | 38,437 | +18 |
| 38,463 | 38,481 | +18 |
| 38,507 | 38,525 | +18 |

The RO/RC heterodimer intermediate, present in significant quantity in Compound 3 (FIG. 26), dissociates into a 1:1 mixture of the two different RO and RC species during LC-MS, which generates both RC and RO PEG polymer series masses. In the heterodimer, these are separated from each other by 18 Da, as one homodimer is RO form (+18 Da), and one is RC. This identity of the RC/RO heterodimer was confirmed by enriching the heterodimeric component of Compound 3 by SCX chromatography, followed by intact mass analysis. The same mass pattern can be generated by combining Compound 3 and Compound 7 in a 1:1 ratio, as would be expected as the two conjugates dissociate into a 1:1 RC/RO mixture during LC-MS analysis.

### Example 15

### In Vivo Study: Single Dose PK Study in Mice

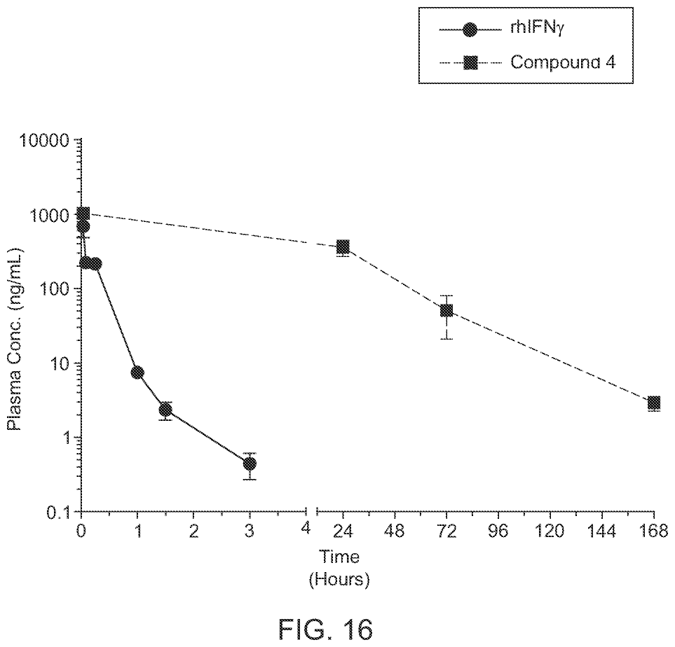

Fox Chase SCID mice (n=3/group) bearing subcutaneous colorectal HT29 tumor were administered a single intravenous dose of rhIFN-γ (control) at 0.1 mg/kg or of Compound 4 at a dose of 0.1 mg/kg. Following administration, blood samples were collected at various time points post administration (rhIFN-γ: 0.033 hours, 0.083 hours, 0.25 hours, 1 hours, 1.5 hours, 3 hours, 24 hours, 72 hours, 168 hours; Compound 4: 0.033 hours, 24 hours, 72 hours, 168 hours). Tumor samples were also collected at 24, 72, and 168 hours for pharmacodynamic analysis of drug action on tumor cells by flow cytometry. Functional marker induction was quantified by mean fluorescence intensity. Finally, at each time point, plasma concentration of drug was determined by ligand binding assay with the results shown in FIG. 16.

As shown in FIG. 16, initial plasma concentrations of Compound 4 and rhIFN-γ were similar. However, Compound 4 maintained measurable concentrations in plasma over an extended period of time, e.g., for greater than 1 week (solid squares), with a T_{1/2} of about 20 hours, in contrast to the rapid drop in plasma levels observed for non-long acting rhIFN-γ (solid circles) following administration.

### Example 16

### Signaling of IFN-γ Mutein Conjugates In Vitro

STAT1 is the major signaling molecule of IFN-γ signaling pathway. The potency of rhIFN-γ and rhIFN-γ conjugates on human monocyte subpopulations CD14⁺CD16⁻ (Classical), CD14⁺CD16⁻ (Intermediate), and CD14^{lo}CD16^{hi} (Non-classical) was determined using a Phospho-STAT1(pY701) dose response assay. Fresh human PBMCs were supplied by AllCells. 1×10⁶ cells were incubated for 15 minutes at 37° C. with the indicated concentrations (20,000 ng/mL-0.000565 ng/mL serial dilutions) of rhIFN-γ or one of conjugates: Compound 1(mPEG-MAL-10K-M135C-rhIFN-γ), Compound 2 (mPEG-MAL-20K-M135C rhIFN-γ), Compound 3 (mPEG2-MAL-20K-M135C-rhIFN-γ), or Compound 4 (mPEG2-MAL-40K-M135C-rhIFN-γ). The cells were fixed using BD Cytofix and permeabilized using BD Perm buffer III, and stained with antibodies for CD14, CD16, and phosphorylated STAT1(pY701) before analysis by flow cytometry. Concentration-response relationships were used to calculate the EC50 using 4-parameter fitting with the results shown in Table 7.

TABLE 7
% pSTAT1 and pSTAT1 MFI in CD14⁺CD16⁻ (Classical),
CD14⁺CD16⁺(Intermediate), and CD141^{lo}CD16^{hi} (Non-classical)

| | EC50 (ng/mL)-% pSTAT1 | | | EC50 (ng/mL)-MFI | | |
|---|---|---|---|---|---|---|
| | | | Non- | | | Non- |
| Compound | Classical | Intermediate | Classical | Classical | Intermediate | Classical |
| rhIFN-γ | 0.035 | 0.015 | 0.018 | 0.3 | 0.24 | 0.26 |
| Compound 1 | 0.222 | 0.084 | 0.106 | 2.16 | 0.85 | 0.99 |
| Compound 2 | 1.449 | 0.179 | 0.367 | 7.62 | 4.28 | 4.57 |
| Compound 3 | 14.010 | 4.379 | 4.844 | 64.24 | 32.73 | 34.96 |
| Compound 4 | 32.610 | 10.070 | 11.730 | 152.1 | 84.24 | 97.48 |

Based upon the data, it appears that rhIFN-γ is ˜10 to 1000-fold more potent than the rhIFN-γ conjugates for pSTAT1 induction. The order of potency was rhIFN-γ>10k PEG-M135C rhIFN-γ (linear)>20k PEG-M135C rhIFN-γ (linear) >>20k PEG2-M135C rhIFN-γ (branched) >40k PEG2-M135C rhIFN-γ (branched).

### Example 17

### Signaling of IFN-γ Mutein Conjugates In Vitro

### In Vitro Potency Assay (Human Colorectal Tumor Epithelial Cell) Based on STAT1 Phosphorylation and in HT-29 Cells

In the phospho-STAT1 assay following receptor binding, IFN-γ receptor engagement by IFN-γ or PEGylated conjugates results in downstream cell signaling and activation of signal transducer and activator of transcription 1 protein (STAT1) by phosphorylation to promote transcription of anti-proliferative genes and induce expression of surface antigen presenting proteins. The activation of phospho-STAT1 in response to reference or sample treatment for 20 minutes was measured in HT-29 tumor cells, a human colorectal epithelial cell line, using the Phospho-STAT1 (Tyr701) cellular assay (Cisbio, Codolet, France).

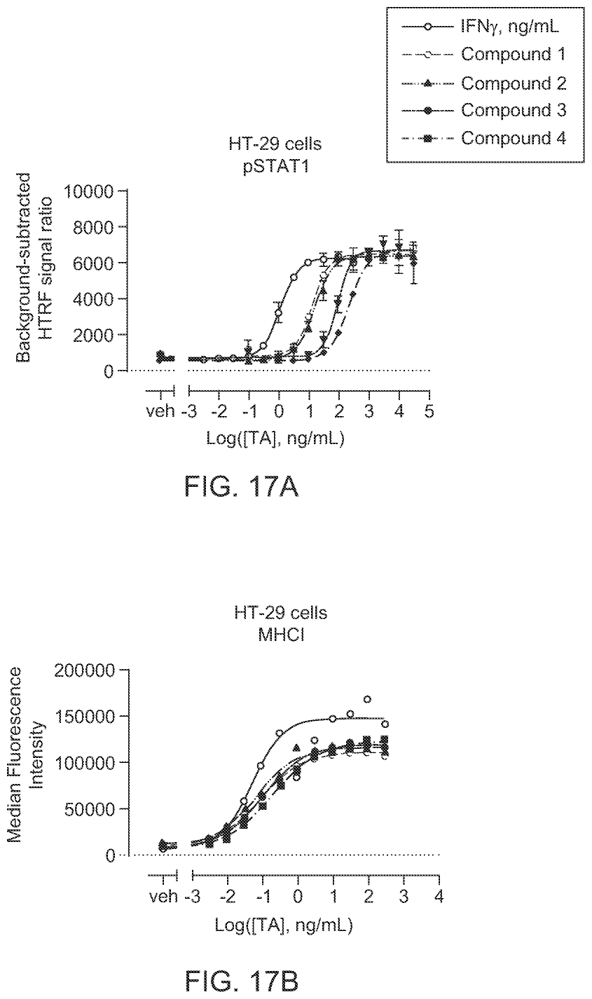

Cultures of HT-29 cells were maintained under ATCC recommendations (Manassas, Virginia). In preparation for the phosho-STAT1 assay, cells were detached from culture flasks using Accutase treatment (Sigma-Aldrich, St. Louis, MO) and seeded in complete growth medium in 96-well plate at 200,000 cells/well. Dilutions of the test articles were prepared in appropriate buffer immediately prior to assay. Stimulation of HT-29 cells was initiated by transfer of 6× test article solutions to duplicate wells containing cells, and plates were incubated at 37° C., 5% CO₂ for 20 minutes. Reaction was stopped by cell lysis and phospho-STAT1 in cell lysate was measured using the Cisbio Phospho-STAT1 (Tyr701) cellular assay kit (Cisbio, Codolet, France) with the results shown in FIGS. 17A-17B. Concentration-response relationships were used to calculate EC₅₀ values.

### Potency Assay (Human Colorectal Tumor Epithelial Cell) Based on MHCI, MHCII, and PD-L1 Induction in HT-29 Cells

In the cellular surface marker expression assay, engagement of IFN-γ receptor by IFN-γ or PEGylated conjugates results in upregulation of surface antigen presenting molecules such as MHCI and MHCII as well as immune-evading molecules such as PD-L1. Surface expression of these membrane proteins following reference or sample treatment for 24 hours is measured in HT-29 tumor cells using flow cytometry.

Cultures of HT-29 cells were maintained under ATCC recommendations (Manassas, VA). In preparation for the phosho-STAT1 assay, cells were detached from culture flasks using Accutase treatment (Sigma-Aldrich, St. Louis, MO) and seeded in complete growth medium in 24-well plate at 200,000 cells/well. Dilutions of the test articles were prepared in appropriate buffer immediately prior to assay. Stimulation of HT-29 cells was initiated by transfer of 11× test article solutions to wells containing cells and plates were incubated at 37° C., 5% CO₂ for 24 hours. The cells were then detached using Accutase and stained with live/dead free amine-binding dye and antibodies for MHCI, MHCII, and PD-L1 before analysis by flow cytometry. Concentration-response relationships were used to calculate EC₅₀ values.

TABLE 8
in vitro signaling potencies of wildtype IFN-γ and related proteins

| | EC50 in HT-29 cells, ng/mL (N) | | | | | | | |
|---|---|---|---|---|---|---|---|---|
| rhIFN-γ variant | pSTAT1 | | MHCI | | MHCII | | PD-L1 | |
| wt (rhIFN-γ-1b) | 1.0 | (2) | 0.079 | (4) | 0.53 | (4) | 0.69 | (4) |
| M135C mutein | 1.4 | (2) | — | | — | | — | |
| N98C mutein | 1.7 | (2) | — | | — | | — | |
| S66C mutein | 1.5 | (2) | — | | — | | — | |
| Compound 1 | 11 | (2) | 0.099 | (2) | 0.83 | (2) | 1.1 | (2) |
| M135C 10K linear | | | | | | | | |
| Compound 2 | 14 | (2) | 0.076 | (2) | 0.70 | (2) | 1.2 | (2) |
| M135C 20K linear | | | | | | | | |
| Compound 3 | 89 | (2) | 0.17 | (3) | 1.8 | (3) | 2.2 | (3) |
| M135C 20K | | | | | | | | |
| branched (RC) | | | | | | | | |
| Compound 4 | 180 | (2) | 0.30 | (3) | 3.1 | (3) | 3.6 | (3) |
| M135C 40K | | | | | | | | |
| branched | | | | | | | | |
| Compound 6 | 1.2 | (2) | 0.047 | (1) | 0.86 | (1) | 1.3 | (1) |
| N98C 20K | | | | | | | | |
| branched | | | | | | | | |
| Compound 5 | 1.3 | (2) | 0.023 | (1) | 0.27 | (1) | 0.37 | (1) |
| S66C 20K | | | | | | | | |
| branched | | | | | | | | |
| Compound 7 (1) | — | | 5.3 | (1) | 150 | (1) | 190 | (1) |
| M135C 20K | | | | | | | | |
| branched (RO) | | | | | | | | |
| Compound 7 (2) | 969 | (2) | — | | — | | — | |
| M135C 20K | | | | | | | | |
| branched (RO) | | | | | | | | |

Table 8 shows in vitro signaling potencies of wildtype (wt) IFN-γ (IFN-γ-1b) and related proteins as measured by phosphorylation of STAT1 and resulting upregulation of surface antigen-presenting molecules MHCI and MHCII and immunomodulatory PD-L1. The desired signaling profile is a conjugate that is less potent than wt IFN-γ in pSTAT1 and PD-L1, but of similar potency in MHCI induction.

FIGS. 17A-17B illustrate in vitro activities of select IFN-γ conjugates, Compounds 1-4, as measured by phospho-STAT1 signaling (FIG. 17A) and downstream surface expression of MHCI molecules (FIG. 17B) in HT-29 tumor cells. A conjugate having a desirable signaling profile is one that is less potent than wt IFN-γ in pSTAT1 and PD-L1, but of similar potency in MHCI induction.

### Example 18

### In Vivo Study: PK/PD Study in Mice HT29 Xenograft Model—PD Markers Induction on Tumor Cells

Fox Chase SCID mice were inoculated subcutaneously with 5×10⁶ HT29 cells. Seven days later, mice were distributed into 4 treatment groups (n=4 groups): vehicle, rhIFN-γ repeat high dose (0.1 mg/kg), Compound 4 single high dose (0.1 mg/kg), Compound 4 single low dose (0.01 mg/kg). Vehicle (PBS) and Compound 4 were administered as a single intravenous dose of 0.01 mg/kg (FIGS. 18A-18C, Compound 4 low dose) and 0.1 mg/kg (FIGS. 18A-18C, Compound 4 high dose) on DO while rhIFN-γ was administered as a once daily intravenous dose of 0.1 mg/kg for 5 consecutive days (FIGS. 18A-18C, IFN-γ repeat high dose). Following administration, blood samples were collected at various time points post-administration for pharmacokinetic evaluation. Tumor samples were also collected at several time points (24 hours, 72 hours, 168 hours) for pharmacodynamic analysis of drug action on tumor cells by flow cytometry. Induction of functional markers (HLA-ABC (HLA-A, HLA-B, and HLA-C MHC-class I antigens), HLA-DR/DP/DQ (HLA-DR, HLA-DP, and HLA-DQ MHC-class II antigens) and PD-L1) on tumor cells was quantified by mean fluorescence intensity (MFI).

Additional conjugates (Compound 2, Compound 3 and Compound 1) were administered as a single intravenous dose of 0.003 mg/kg or 0.03 mg/kg (FIGS. 19A-19C) on DO and tumor samples were collected for pharmacodynamic evaluation at various timepoints post administration (72 hours, 168 hours, 240 hours).

Tumor cells were defined using mCD45−hEGFR+ markers combination. Following dosing and tumor dissociation, tumor samples were acquired on the Fortessa flow cytometer running FACS DIVA software. Flowjo software was used for analysis and HLA-ABC, HLA-DR/DP/DQ and PD-L1 mean MFI were plotted using prism.

Results in FIGS. 18A-18C illustrate the ability of Compound 4 to induce the expression of HLA-ABC (FIG. 18A), HLA-DR/DP/DQ (FIG. 18B) and PD-L1 (FIG. 18C) on tumor cells. Compound 4 induced a dose-dependent increase in surface expression of all three markers on tumor cells. Induction of HLA-ABC (FIG. 18A) in all dose groups and HLA-DR/DP/DQ in high dose groups (FIG. 18B) was sustained over 7 days (168 hours) post administration while PD-L1 expression was back to vehicle level by day 7 in all dose groups. Single low dose of Compound 4, a long acting rhIFN-γ molecule, drove an induction of HLA-ABC on tumor cells equivalent to daily intravenous administration of high dose rhIFN-γ.

Single dose of Compounds 1, 2, and 3 also induced a dose dependent increase in the expression of HLA-ABC (FIG. 19A), HLA-DR/DP/DQ (FIG. 19B) and PD-L1 (FIG. 19C) on the surface of tumor cells in this xenograft model. HLA-ABC induction (FIG. 19A) was sustained through day 7 (168 hours) post 0.03 mg/kg dose of Conjugates administration. While HLA-ABC (FIG. 19A) expression on tumor cells was induced by the single low 0.003 mg/kg dose of the compounds 72 hours post administration, HLA-DR/DP/DQ (FIG. 19B) and PD-L1 (FIG. 19C) were not induced at this dose.

As seen in FIG. 18A, a 10× lower single dose of Compound 4 (Compound 4, low dose) yielded sustained MHCI induction that was comparable to daily (for five days) dosing of the non-polymer modified rhIFN-γ.

### Example 19

### Receptor Affinity Evaluation of IFN-γ-1b Cysteine Muteins

The affinities of IFN-γ-1b protein, the M135C, S98C, and S66C cysteine muteins, and Compounds 1, 2, and 3 were measured using Surface Plasmon Resonance (“SPR”) using a BAcore™ SPR system. Briefly, the surface of a BAcore™ CM5 sensor chip was coated with anti-human IgG antibody comprising IFNγR1 fused to the constant region of the antibody IgG₁.

Each of the IFN-γ-b protein, the M135C, S98C, and S66C cysteine muteins, and Compounds 1, 2, and 3 were separately injected onto the sensor chip which was coated with IFNγR1. The affinities were measured by determining the kinetic on-rate (kₒₙ) and the kinetic off-rate (k_{off}) rates separately, and the ratio between kₒₙ and k_{off} was used to calculate the K_{d} values with the results shown in Table 9.

TABLE 9
SPR results of IFN- γ, cysteine muteins,
and conjugate binding to IFNγR1

| | Results | | |
|---|---|---|---|
| Analyte | kₒₙ (M⁻¹S⁻¹) | k_{off} (S⁻¹) | K_{D} (M) |
| rhIFN-γ-1b | 1.07E+07 | 7.01E−04 | 6.55E−11 |
| M135C | 1.66E+06 ± | 8.64E−04 ± | 1.23E−10 ± |
| | 1.63E05 | 8.0E−04 | 4.14E−11 |
| N98C | 6.81E+06 ± | 9.34E−04 ± | 4.44E−10 ± |
| | 5.45E06 | 7.22E−04 | 6.5E−11 |
| S66C | 2.57E+06 ± | 1.37E−03 ± | 5.34E−10 ± |
| | 1.77E04 | 6.13E−04 | 2.35E−10 |
| Compound 1 | 1.61E+06 ± | 2.21E−03 ± | 1.42E−09 ± |
| mono-mPEG-MAL-10K- | 4.63E05 | 1.02E−04 | 3.44E−10 |
| rhIFN-γ-1b-M135C | | | |
| Compound 2 | 6.37E+05 ± | 1.06E−03 ± | 1.88E−09 ± |
| mono-mPEG-MAL-20K- | 2.96E05 | 5.30E−05 | 9.57E−10 |
| rhIFN-γ-1b-M135C | | | |
| Compound 3 | 3.21E+05 ± | 1.57E−03 ± | 4.90E−09 ± |
| mono-mPEG2-ru-MAL- | 5.66E03 | 3.54E−05 | 1.96E−10 |
| 20K-rhIFN-γ-1b-M135C | | | |

WT IFN-γ-1b (Actimmune®) has a K_{D} of 65.5 pM. IFN-γ-M135C showed a slight decrease in affinity to 123 pM. The affinities of PEG conjugates at this site depended on the size and the structure of the PEG. Attaching a linear 10 kDa PEG (Compound 1) decreased the K_{D} to 1.42 nM, an approximately 10-fold decrease. Attaching a larger molecular weight linear 20 kDa PEG (Compound 2) had a similar but slightly lower affinity (than the 10 kDa PEG) of 1.88 nM. Finally, attaching a branched 20 kDa PEG (Compound 3) had the lowest affinity among these conjugates at 4.90 nM. All three conjugates are considered very active.

### Example 20

### In Vivo PD Study in a Mice B16F10 Syngeneic Model—PD Markers Induction on Tumor Cells; T Cells Response

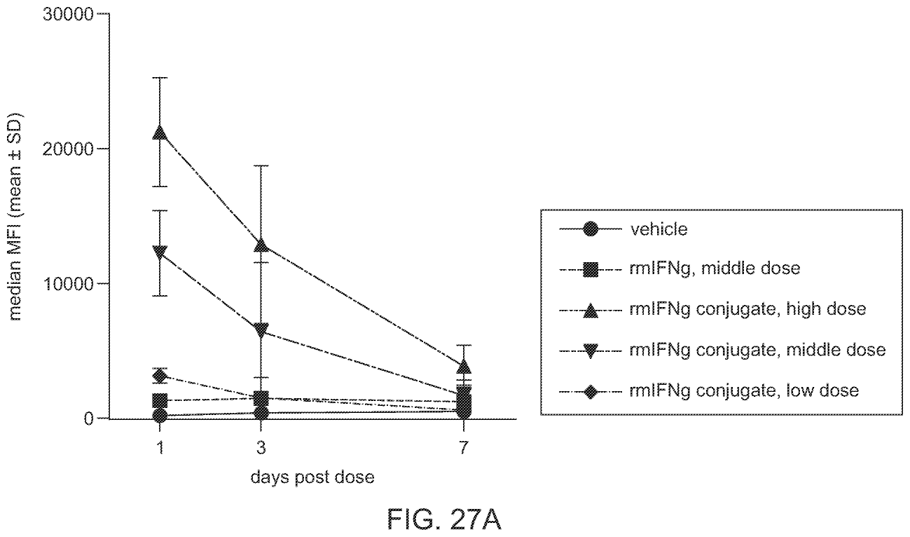

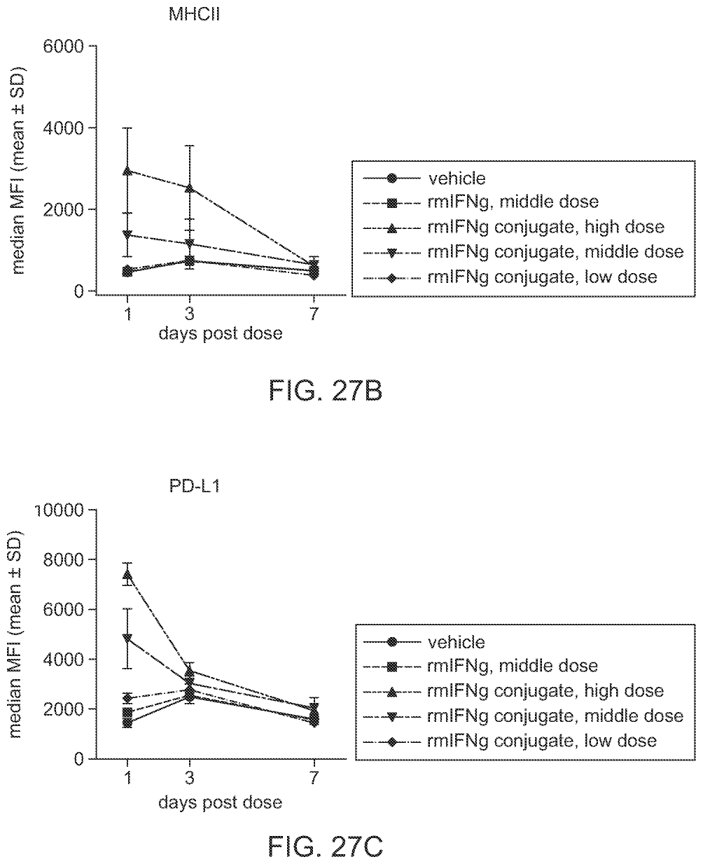

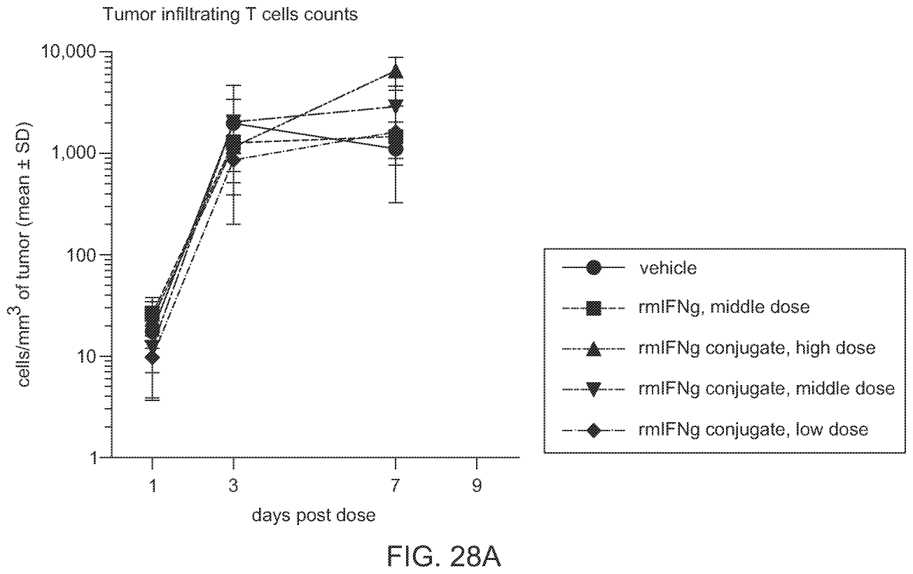

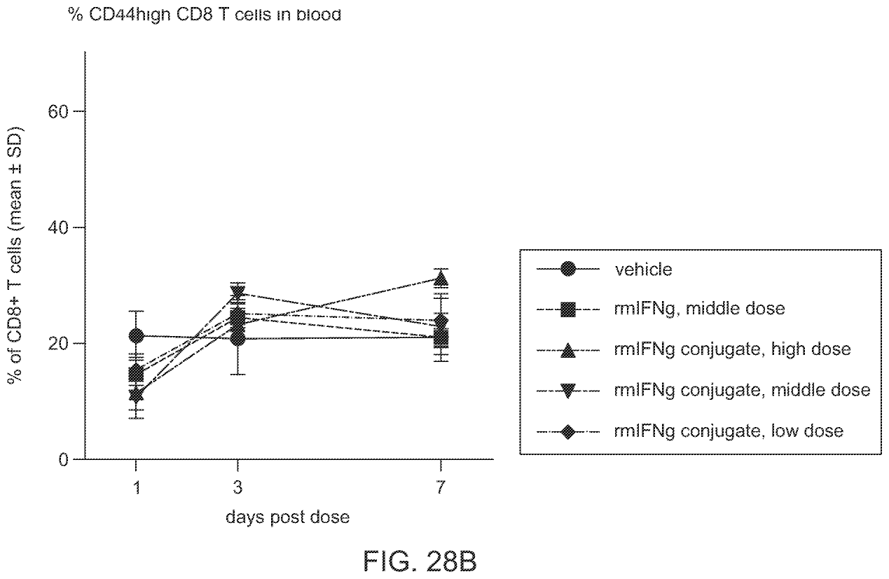

Compound 10 was prepared essentially as described in Example 5 using a recombinant mouse IFN-γ sequence (SEQ ID NO:9; rmIFN-γ). C57Black 6 mice were inoculated subcutaneously with 1.2×10⁶ B16F10 melanoma cells. Seven days later, mice were distributed into 5 treatment groups (n=4 groups): vehicle, rmIFN-γ (SEQ ID NO:9), Compound 10 at high dose, Compound 10 at middle dose, and Compound 10 at low dose. All compounds were administered as a single dose on day 0 by the intravenous route. rmIFN-γ was dosed at a middle dose of 0.3 mg/kg while Compound 10 was administered at a high dose of 0.6 mg/kg, at a middle dose of 0.3 mg/kg, or at a low dose of 0.1 mg/kg. Doses of Compound 10 refer to the amount of rmIFN-γ protein. Following administration, blood and tumor samples were collected at days 1, 3 and 7 for pharmacodynamic analysis of drug action on the tumor cells as well as for T cells response determination by flow cytometry. Induction of functional markers (MHCI, MHCII and PD-L1) on tumor cells was quantified by mean fluorescence intensity (MFI) as shown in FIGS. 27A-27C. The number of T cells (per volume of tumor as well as fraction positive for marker of activation (CD44high) were also investigated and are shown in FIGS. 28A and 28B, respectively.

Tumor cells were defined using mCD45− and side scatter combination and T cells as CD45+CD3+. Following dosing, tissue collection and tumor dissociation, blood and tumor samples were acquired on the Fortessa flow cytometer running FACS DIVA software. Flowjo software was used for analysis and PD marker induction on tumor cells as well as T cells response were plotted using Prism.

Results in FIGS. 27A-27C illustrate the ability of the rmIFN-γ conjugate, Compound 10, to induce the expression of MHCI (FIG. 27A), MHCII (FIG. 27B), and PD-L1 (FIG. 27C) on tumor cells. Compound 10 induced a dose-dependent increase in surface expression of all three markers on tumor cells on day 1 post administration. Induction of MHCI (FIG. 27A) at the high and middle dose groups and MHCII (FIG. 27B) at the high dose groups was sustained through day 3 post administration. Minimal induction of all three markers was observed after administration of rmIFN-γ, without polymer modification, at the middle, 0.3 mg/kg dose group.

FIGS. 28A and 28B illustrate the PD response of immune effector T cells following administration of Compound 10, the rmIFN-γ conjugate. Tumor infiltrating T cells count normalized to tumor volume (FIG. 28A) were increased on day 7 post dose after administration of Conjugate 10 at the high dose (0.6 mg/kg). In the blood, the fraction of CD44high CD8+ T cells (FIG. 28B) which represent a memory/memory effector phenotype was also increased on day 7 after administration of Conjugate 10 at the high dose (0.6 mg/kg).

Administration of Compound 10 drove dose dependent MHCI and MHCII induction on tumor cells in the B16F10 melanoma model. An increase of tumor infiltrating T cells and an increase in the fraction of memory like CD8+ T cells in the blood of animals administered a high dose of Compound 10 was also observed.

### Example 21

### In Vivo Administration of rmIFN-γ Conjugate in Combination with a PD-1 Antagonist or a PD-L1 Antagonist in a B16F10 Melanoma Tumor Model

Studies were conducted to evaluate and compare the anti-tumor response of Compound 10, a long acting IFN-γ mouse conjugate (mono-mPEG2-ru-MAL-20K-rmIFN-γ), in combination with a mouse PD-1 antagonist (clone RPM1-14 anti-mouse PD-1 antibody available from BioXcell) or a mouse PD-L1 antagonist (clone 10F.9G2 anti-mouse PD-L1 antibody obtained from BioXcell) in a subcutaneous B16F10 melanoma tumor model when compared to immunotherapy with Compound 10 alone or the single agent PD-1 antagonist or PD-L1 antagonist. The sequence of the mouse IFN-γ used for preparing Compound 10 is SEQ ID NO:9, which is the wild type mouse IFN-γ including an N-terminal methionine for recombinant E. coli expression. The wild type mouse IFN-γ sequence includes a C-terminal cysteine which was used for conjugation of the polymer.

In vivo model: Mice used were −8 weeks old female C57B1I6 strain were implanted with 0.75 million B16F10 melanoma tumor cells on one flank. Cells were allowed to mature into tumors for 7 days reaching a volume of 75-150 mm³ volume (day 0).

Dosing: Compound 10 was administered systemically by intravenous injection at 0.6 mg/kg. PD-1 antagonist or PD-L1 antagonist was administered intraperitoneally at 400 μg. Compound 10 was administered on days 0 and 7 for a total of 2 doses while the PD-1 antagonist or PD-L1 antagonist was administered on days 0, 4, 7, 10 for a total of 4 doses. A vehicle was administered to a vehicle group intraperitoneally with a 100 μl of phosphate-buffered saline (PBS) on days 0, 4, 7, 10 for a total of 4 doses. Doses of Compound 10 refer to the amount of rmIFN-γ protein.

Measurements: Tumor volumes were determined by caliper measurements 2-3 times per week and calculated using formula: L×W²/2 where L is tumor length and W is tumor width. Animals were removed from the study when tumor volume reached 2000 mm3 or when animals met humane removal endpoints.

Results: Data is provided in the Table 10. Administration of Compound 10 as a single agent was effective in slowing tumor growth while administration of the PD-1 antagonist or the PD-L1 antagonist as single agents showed minimum efficacy. Doublet combination therapy of Compound 10 with the PD-1 antagonist or the PD-L1 antagonist provided an increased efficacy in slowing tumor growth over single agent administration of any of Compound 10, the PD-1 antagonist, or the PD-L1 antagonist.

TABLE 10
Tumor volume measurements

| Days | | | | | | |
|---|---|---|---|---|---|---|
| after | | | | | | |
| treatment | | Compound | | | Compound | Compound |
| start | Vehicle | 10 | aPD-1 | aPD-L1 | 10 + aPD-1 | 10 + aPD-L1 |
| 0 | + | + | + | + | + | + |
| 3 | + | + | + | + | + | + |
| 6 | ++ | ++ | ++ | ++ | + | + |
| 8 | ++++ | ++ | +++ | +++ | ++ | ++ |
| 10 | NA | +++ | ++++ | ++++ | +++ | ++ |
| 13 | NA | NA | NA | NA | ++++ | ++++ |

Mean tumor volumes per group in mm³ are provided as follows:
0≤ + <500
500≤ ++ <1000
1000≤ +++ <1500
1500≤ ++++ <2000
NA: 2 or more animals/group removed due to reaching maximum tumor volume or humane endpoint.

The combination treatment with rmIFN-γ conjugate with a PD-1 antagonist or a PD-L1 antagonist showed significant improvement over the vehicle treatment by slowing tumor growth in treated animals. The vehicle group had no surviving animals by end of study at day 13. All animals in the vehicle group were removed from the study due to reaching limiting tumor volume or humane endpoint.

At day 7, treatment with the rmIFN-γ conjugate provided a tumor growth inhibition (TGI) of 58% as compared to treatment with the vehicle. Treatment with the PD-1 antagonist or the PD-L1 antagonist provided similar results to each other with at TGI of 27% or 28% as compared to vehicle, respectively. Combination treatment with the rmIFN-γ conjugate and either of the PD-1 antagonist or the PD-L1 antagonist provided significant improvement over the vehicle or treatment with either of the PD-1 antagonist or the PD-L1 antagonist (68% TGI or 66% TGI).

### Example 22

### In Vitro Potency in an HT-29 Human Colorectal Tumor Epithelial Cell Line Assay Based on Intracellular STAT1 Phosphorylation

IFN-γ receptor engagement by IFN-γ or pegylated conjugates results in downstream cell signaling mediated by signal transducer and activator of transcription 1 protein (STAT1) activation by phosphorylation, to promote transcription of anti-proliferative genes and induce expression of antigen presenting proteins. The activation via phosphorylation of STAT1 in response to reference or sample treatment for 20 minutes is measured in HT-29 tumor cells, a human colorectal epithelial cell line, by quantifying Phospho-STAT1 (pY701) in either a homogeneous time-resolved fluorescence cellular assay (Cisbio, Codolet, France) or by anti-pSTAT1 (pY701) antibody staining via flow cytometry (BD Biosciences, Franklin Lakes, New Jersey, USA).

Cultures of HT-29 cells were maintained under ATCC recommendations (Manassas, Virginia). In preparation for the phosho-STAT1 assay, cells were detached from culture flasks using Accutase treatment (Sigma-Aldrich, St. Louis, MO) and seeded in complete growth medium in 96-well plate at 200,000 cells/well. Dilutions of the test articles were prepared in appropriate buffer immediately prior to assay. Stimulation of HT-29 cells was initiated by transfer of 6× test article solutions to duplicate wells containing cells, and plates were incubated at 37° C., 5% CO₂ for 20 minutes. Reaction was stopped by cell lysis and phospho-STAT1 in cell lysate was measured using the Cisbio Phospho-STAT1 (pY701) cellular assay kit (Cisbio, Codolet, France), or by cell fixation with formaldehyde followed by methanol permeabilization and staining with anti-pSTAT1(pY701) antibody for flow cytometry (BD Biosciences, Franklin Lakes, New Jersey, USA). Concentration-response relationships were used to calculate EC₅₀ values.

### Example 23

### Assay of Heparin Binding In Vitro

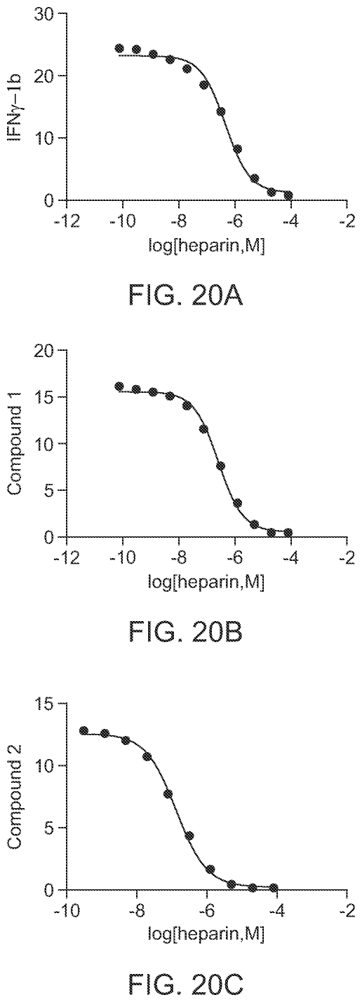

Heparin binding of IFN-γ-M135C, Compound 1, or Compound 2 was assayed in a competition assay order to determine if binding of hIFN-γ by heparin can be mitigated and, thus, if the inhibition of hIFN-γ by heparin can be mitigated. In this assay, the surface of a CM5 sensor chip was activated with EDC/NHS chemistry. An anti-human IgG antibody was covalently attached to the activated CM5 surface. Interferon gamma receptor 1 (IFNγR1) was coated on the surface of a sensor chip, that is fused to the Fc region of the antibody. The sensor chips were incubated with IFN-γ-M135C, Compound 1 or Compound 2 in the presence of heparin. The binding of IFN-γ-M135C, Compound 1 or Compound 2 were measured in the presence of increasing of heparin concentration acting as a competitive ligand. The Ki in nM was calculated with the results provided in Table 12 and shown graphically in FIGS. 20A-20C. Heparin was calculated to have a reasonable affinity to IFN-γ-M135C. Attaching a polymer such as a linear 10 kDa (Compound 1) or a linear 20 kDa PEG (Compound 2) decreased the binding affinity of heparin affinities by about 1.5 to 2.5 fold, respectively.

TABLE 12
Calculated Ki of heparin binding to
Interferon-γ-M135C or its conjugates

| | IFN-γ- | | |
|---|---|---|---|
| | M135C | Compound 2 | Compound 1 |
| fold | — | 1.6 | 2.4 |
| change | | | |

### Example 24

### Assay of Heparin Binding In Vitro

The effect of PEGylation of IFN-γ on heparin binding to the protein was assessed by directly measuring binding of rhIFN-γ, Compound 1, Compound 2, Compound 3, or Compound 4 to heparin. Biotinylated heparin was coated on the surface of a sensor chip SA coated with streptavidin. The sensor chips were incubated with rhIFN-γ, Compound 1, Compound 2, Compound 3, or Compound 4. The kinetic on-rate (kₒₙ) and kinetic off-rate (k_{off}) were measured separately by Surface Plasmon Resonance (“SPR”) using a Cytiva BIAcore™ T200 SPR system. The equilibrium dissociation constant (K_{D}) was calculated for each compound from the measured kₒₙ and k_{off}. The results are provided in Table 13.

TABLE 13
K_{D} values of Interferon or its conjugates binding to heparin

Results

| Analyte | kₒₙ (M⁻¹S⁻¹) | k_{off} (S⁻¹) | K_{D} (M) |
|---|---|---|---|
| rhIFN-γ-1b wt | 5.09E+06 | 5.98E−03 | 1.17E−08 |
| rhIFN-γ-1b-M135C | 4.02E+05 | 1.28E−03 | 3.18E−09 |
| Compound 1 | 6.26E+04 | 3.96E−03 | 6.33E−08 |
| mono-mPEG-MAL- | | | |
| 10K-rhIFN-γ-1b- | | | |
| M135C | | | |
| Compound 2 | 1.08E+05 | 4.60E−03 | 4.26E−08 |
| mono-mPEG-MAL- | | | |
| 20K-rhIFN-γ-1b- | | | |
| M135C | | | |
| Compound 3 | 3.79E+03 | 1.28E−02 | 1.69E−07 |
| mono-mPEG2-ru- | | | |
| MAL-20K-rhIFN-γ- | | | |
| 1b-M135C | | | |
| Compound 4 | 4.66E+04 | 1.98E−02 | 4.24E−07 |
| Mono-mPEG2-MAL- | | | |
| 40k-rhIFN-γ-1b- | | | |
| M135C | | | |

The affinity of the wild-type (wt) IFN-γ-1 b to heparin (K_{D}) was 11.7 nM. M135C variant possessed a slightly higher binding affinity of 3.18 nM. PEGylation at the M135C site, however, drastically reduced the binding affinities of the conjugates to heparin. Conjugating a linear 10 kDa PEG or a 20 kDa PEG reduced the heparin binding affinity of the conjugates to 63.3 nM and 42.6 nM, Compounds 1 and 2, respectively. Compound 3, a branched 20 kDa conjugate, had a markedly reduced binding affinity to heparin at 170 nM. Finally, Compound 4, a branched 40 kDa conjugate, had a further reduced heparin binding affinity at 424 nM. PEGylation of the mutein IFN-γ protein markedly reduced the heparin binding affinity for the conjugates as compared to the wild-type IFN-γ-1b or the rhIFN-γ-1b M135C mutein.

### Example 25

### Assay of Heparin Binding In Vitro

In order to assess the effect of maleimide ring opening, Compound 3 was treated with base and repurified to remove any free IFNg. The resulting compound was ring-open (RO) Compound 7, which was characterized for its ability to bind heparin. Briefly, the surface of a BIAcore™ SA sensor chip coated with streptavidin was coated on the surface with biotinylated heparin. The sensor chips were incubated with Compound 3 or Compound 7. The kinetic on-rate (kₒₙ) and kinetic off-rate (k_{off}) were measured separately by Surface Plasmon Resonance (“SPR”) using a Cytiva BIAcore™ T200 SPR system. The equilibrium dissociation constant (K_{D}) was calculated for each compound from the measured kₒₙ and k_{off}. The results are provided in Table 14.

TABLE 14
K_{D} values of Interferon or its conjugates binding to heparin

| | Free IFN-γ | Results | | |
|---|---|---|---|---|
| Analyte | M135C (%) | kₒₙ (M⁻¹S⁻¹) | k_{off} (S⁻¹) | K_{D} (M) |
| Compound 3 | 0.3 | 4.72E+04 | 8.43E−03 | 1.79E−07 |
| RC Mono-mPEG2- | | | | |
| MAL-20k-rhIFN-γ- | | | | |
| 1b-M135C | | | | |
| Compound 7 | 0.16 | No binding/weak binding | | |
| RO Mono-mPEG2- | | | | |
| MAL-20k-rhIFN-γ- | | | | |
| 1b-M135C | | | | |

Compound 3 bound heparin with a measurable affinity (K_{D}) of 179 nM. In contrast, the binding affinity of Compound 7 to heparin had a reduced affinity as to make a reasonable measurement difficult. The ring opened IFN-γ mutein conjugate exhibited a vastly decreased affinity for heparin binding even as compared to the ring closed version of the conjugate.

Embodiments of the present conjugates, methods, therapeutic combinations and kits include, but are not limited to:

Embodiment 1. A conjugate comprising interferon-gamma (IFN-γ) covalently attached to a water-soluble, non-peptidic polymer at a cysteine residue of the IFN-γ, wherein the IFN-γ is a mutein comprising a cysteine substitution or insertion (“IFN-γ cysteine mutein”).

Embodiment 2. The conjugate of embodiment 1, wherein the conjugate has a structure:

IFN-γ-s-x-POLY Formula I,

wherein IFN-γ is a cysteine mutein of IFN-γ, —S—, is a sulfur atom of the cysteine, X is a spacer moiety, and POLY is a water-soluble, non-peptidic polymer.

Embodiment 3. The conjugate of the combined or separate embodiments 1-2, having a structure:

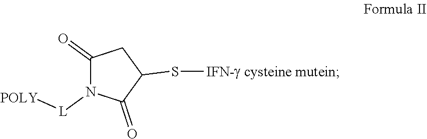

wherein the spacer moiety X comprises

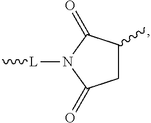

wherein L is a linker comprising one or more atoms selected from carbon, nitrogen, sulfur, oxygen, and combinations thereof, POLY is the water-soluble, non-peptidic polymer; and —S— is a sulfur atom of the cysteine.

Embodiment 4. The conjugate of the combined or separate embodiments 1-3, having a structure:

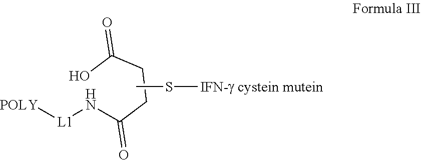

wherein the spacer moiety X comprises

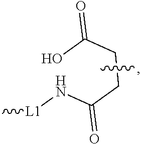

wherein L1 is a linker comprising one or more atoms selected from carbon, nitrogen, sulfur, oxygen, and combinations thereof, POLY is the water-soluble, non-peptidic polymer; and —S— is a sulfur atom of the cysteine.

Embodiment 5. The conjugate of the combined or separate embodiments 1-4, wherein the spacer or linker comprises one or more atoms selected from a carbon-carbon bond, an amide, a carbamate, an amine, an ether, and combinations thereof.

Embodiment 6. The conjugate of the combined or separate embodiments 1-5, wherein the linker L or L1 is ˜(CH₂)ₐ(O)_{b}[C(O)]_{c}(NH)_{d}(CH₂)ₑ(NH)_{f}[C(O)]_{g}(CH₂)ₕ˜, wherein: a is 0-10; b is 0, 1; c is 0, 1; d is 0, 1; e is 0-10; f is 0, 1; g is 0, 1; and h is 0-10, wherein at least one of a, b, c, d, e, f, g, and h is a positive integer.

Embodiment 7. The conjugate of the combined or separate embodiments 1-6, where f is 1, g is 1 and h is 2.

Embodiment 8. The conjugate of the combined or separate embodiments 1-7, where a, b, c, d, and e are zero.

Embodiment 9. The conjugate of the combined or separate embodiments 1-7, wherein a is 3, b is 0, c is 1, d is 1 and e is 2.

Embodiment 10. The conjugate of the combined or separate embodiments 1-9, wherein POLY is linear or branched.

Embodiment 11. The conjugate of embodiment 10, wherein the branched POLY comprises from about 2 to about 10 polymer arms.

Embodiment 12. The conjugate of the combined or separate embodiments 1-11, wherein the water-soluble, non-peptidic polymer is a poly(alkylene oxide).

Embodiment 13. The conjugate of the combined or separate embodiments 1-12, wherein the poly(alkylene oxide) is a poly(ethylene oxide).

Embodiment 14. The conjugate of the combined or separate embodiments 1-13, wherein POLY comprises —(CH₂CH₂O)ₙ—Y or —(OCH₂CH₂)ₙ—Y, wherein Y is selected from a lower alkyl or hydroxyl; and n is an integer ranging from about 45 to about 1818. Embodiment 15. The conjugate of embodiment 14, wherein the lower alkyl is methyl. Embodiment 16. The conjugate of the combined or separate embodiments 1-15, wherein POLY comprises a structure:

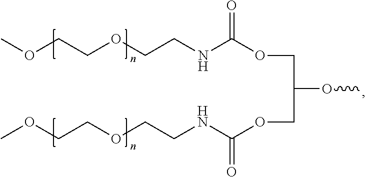

wherein each n is independently an integer ranging from about 45 to about 1818.

Embodiment 17. The conjugate of the combined or separate embodiments 1-16, wherein POLY has a weight average molecular weight of from about 2,000 daltons to about 80,000 daltons.

Embodiment 18. The conjugate of the combined or separate embodiments 1-17, wherein POLY has a weight average molecular weight of from about 2,000 daltons to about 40,000 daltons.

Embodiment 19. The conjugate of the combined or separate embodiments 1-18, wherein POLY has a weight average molecular weight of from about 10,000 daltons to about 40,000 daltons.

Embodiment 20. The conjugate of the combined or separate embodiments 1-19, wherein the IFN-γ cysteine mutein has a sequence having at least 95% sequence identity to a sequence set forth in SEQ ID NO: 4, SEQ ID NO: 5, or SEQ ID NO:6.

Embodiment 21. The conjugate of the combined or separate embodiments 1-20, wherein the IFN-γ cysteine mutein has a sequence having at least 95% sequence identity to SEQ ID NO:1, SEQ ID NO:2, SEQ ID NO:3, or SEQ ID NO: 7 and comprises a cysteine substitution or insertion.

Embodiment 22. The conjugate of the combined or separate embodiments 1-21, wherein the IFN-γ cysteine mutein sequence includes a cysteine residue substituted for at least one amino acid selected from the group consisting of the serine at position 66, the asparagine at position 98, and the methionine at position 135 of SEQ ID NO:3 or SEQ ID NO:7.

Embodiment 23. The conjugate of the combined or separate embodiments 1-22, wherein the cysteine substitution or insertion is located within the interferon gamma receptor 1 (IFNGR1) binding region of the IFN-γ mutein.

Embodiment 24. The conjugate of the combined or separate embodiments 1-23, wherein the cysteine substitution or insertion is located within 1-10 amino acids from either end of the IFNGR1 binding region of the IFN-γ cysteine mutein.

Embodiment 25. The conjugate of the combined or separate embodiments 1-24, wherein the cysteine substitution or insertion is located at the C-terminus of the IFN-γ mutein.

Embodiment 26. The conjugate of the combined or separate embodiments 1-25, wherein the conjugate has a structure selected from:

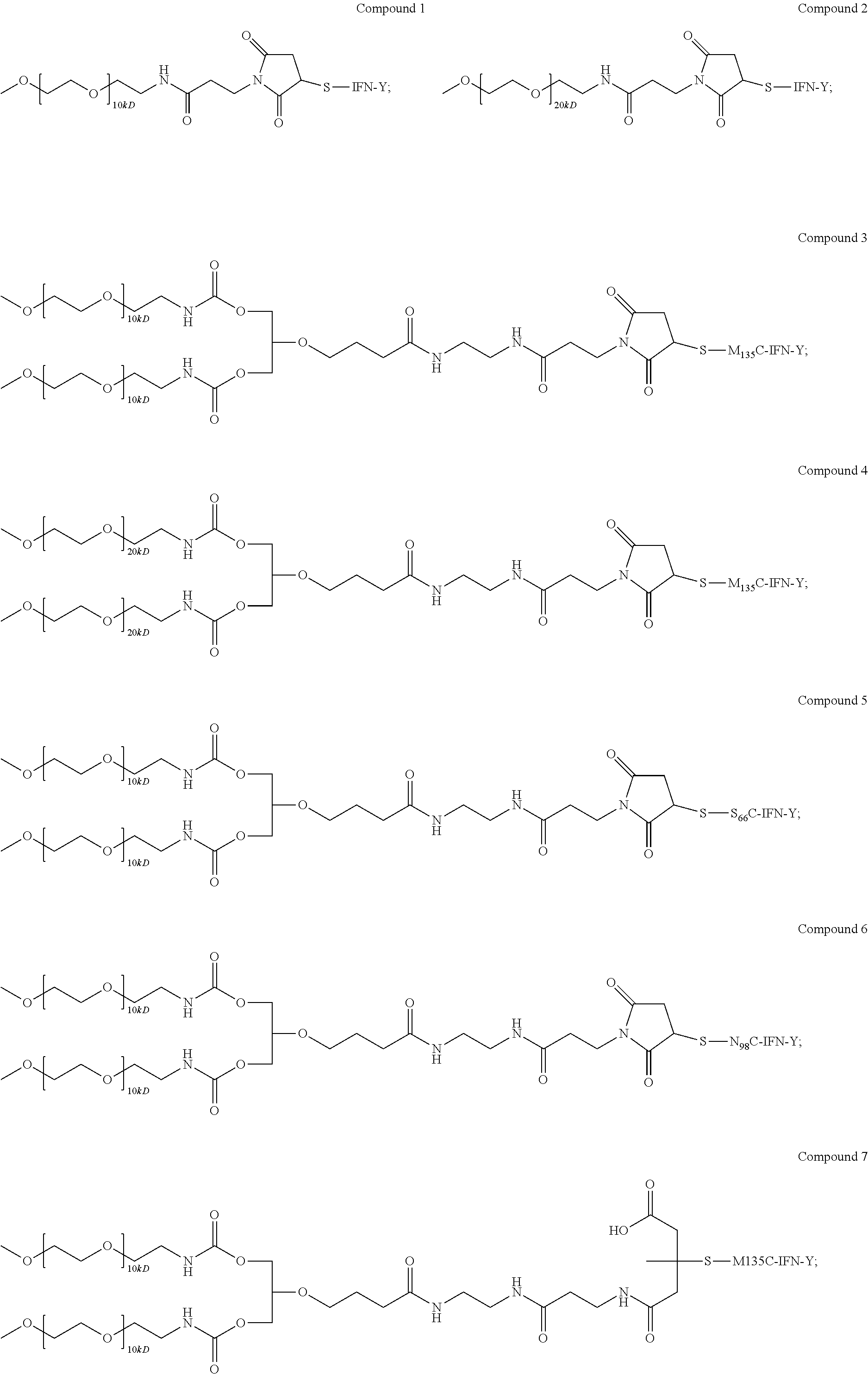

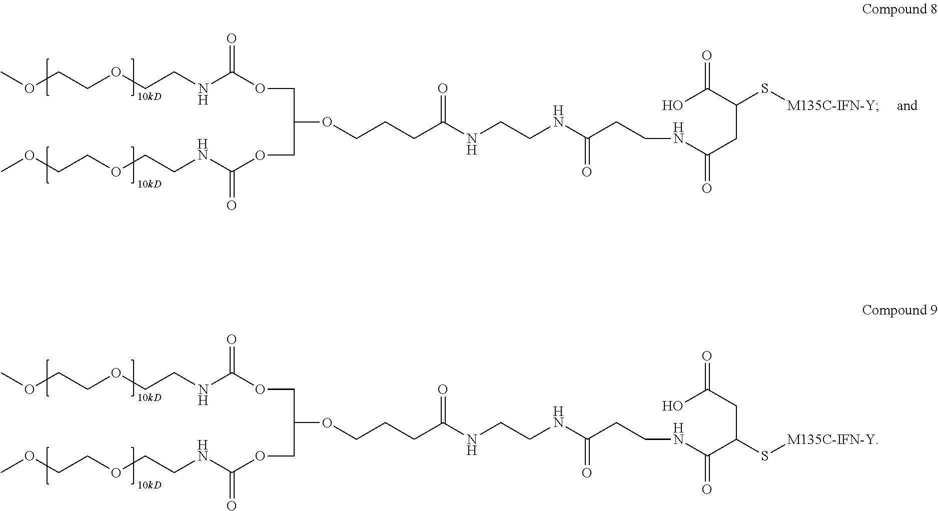

Embodiment 27. The conjugate of the combined or separate embodiments 1-26, having an EC50 value (ng/mL, human PMBCs pSTAT1) that is reduced by at least about 3-fold when compared to the EC50 value (ng/mL, human PMBCs pSTAT1) of the non-polymer modified IFN-γ cysteine mutein and/or unmodified IFN-γ, or at least about 3.5-fold, or at least about 4-fold, or at least about 4.5-fold, or at least about 5-fold, or at least about 5.5-fold, or at least about 6-fold, or at least about 6.5-fold, or at least about 7-fold, or at least about 7.5-fold, or at least about 8-fold, or at least about 8.5-fold, or at least about 9-fold, or at least about 9.5-fold, or at least about 10-fold when compared to the EC50 value (ng/mL, human PMBCs pSTAT1) of the non-polymer modified IFN-γ cysteine mutein and/or unmodified IFN-γ.

Embodiment 28. The conjugate of the combined or separate embodiments 1-27, wherein the conjugate exhibits a reduction in major histocompatibility complex class I (MHCI) induction of no more than about 3-fold as measured by its EC50 value (ng/mL, HT-29 MHCI) when compared to the MHCI induction as measured by EC50 value (ng/mL, HT-29 MHCI) of the non-polymer modified IFN-γ cysteine mutein and/or unmodified IFN-γ, or no more than about 3.5-fold, or no more than about 4-fold, or no more than about 4.5-fold, or no more than about 5-fold when compared to the MHCI induction as measured by EC50 value (ng/mL, HT-29 MHCI) of the non-polymer modified IFN-γ cysteine mutein and/or unmodified IFN-γ.

Embodiment 29. The conjugate of the combined or separate embodiments 1-28, wherein the conjugate exhibits a reduction in major histocompatibility complex class II (MHCII) induction of no more than about 3-fold as measured by its EC50 value (ng/mL, HT-29 MHCII) when compared to the MHCI induction as measured by EC50 value (ng/mL, HT-29 MHCII) of the non-polymer modified IFN-γ cysteine mutein and/or unmodified IFN-γ, or no more than about 3.5-fold, or no more than about 4-fold, or no more than about 4.5-fold, or no more than about 5-fold when compared to the MHCI induction as measured by EC50 value (ng/mL, HT-29 MHCII) of the non-polymer modified IFN-γ cysteine mutein and/or unmodified IFN-γ.

Embodiment 30. The conjugate of the combined or separate embodiments 1-29, exhibiting a reduction in IFNGR1 binding (K_{D}) of at least about 3% when compared to IFNGR1 binding (K_{D}) of the non-polymer modified IFN-γ cysteine mutein and/or unmodified IFN-γ, or at least about 4%, or at least about 5%, or at least about 6%, or at least about 7%, or at least about 8%, or at least about 9%, or at least about 10%. Embodiment 31. The conjugate of the combined or separate embodiments 1-30, having a decrease in heparin binding (Kᵢ) of at least about 1% when compared to heparin binding (Kᵢ) of the non-polymer modified IFN-γ cysteine mutein and/or unmodified IFN-γ, or at least about 2%, or at least about 3%, or at least about 4%, or at least about 5%. Embodiment 32. The conjugate of the combined or separate embodiments 1-31, having a reduction in heparin binding (Kᵢ, nM) of at least about a 1 fold when compared to the heparin binding (Kᵢ, nM) of the non-polymer modified IFN-γ cysteine mutein and/or unmodified IFN-γ, or at least about 1.5-fold.

Embodiment 33. A composition comprising a conjugate of the combined or separate embodiments 1-32, and a pharmaceutically acceptable excipient.

Embodiment 34. The composition of embodiment 33, comprising a conjugate of the combined or separate embodiments 1-32, wherein no more than about 15 mole percent of conjugates comprised in the composition have the following ring-closed structure:

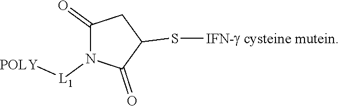

Embodiment 35. A method for treating a subject having a disease that is responsive to treatment with IFN-γ comprising administering to the subject a therapeutically effective amount of the conjugate of the combined or separate embodiments 1-32 or the composition of the combined or separate embodiments 33-34.

Embodiment 36. The method of embodiment 35, wherein the disease is a cancer.

Embodiment 37. The method of the combined or separate embodiments 35-36, wherein the cancer is a liquid cancer.

Embodiment 38. The method of the combined or separate embodiments 35-37, wherein the cancer is a solid cancer.

Embodiment 39. The method of the combined or separate embodiments 35-38, wherein the cancer is selected from small cell lung cancer (SCLC), non-small cell lung cancer (NSCLC), adrenocortical carcinoma, myxoid/round cell liposarcoma, synovial sarcoma, gliosarcoma, fallopian tube cancer, ovarian cancer, renal cell carcinoma (RCC), colorectal cancer, microsatellite instability-high cancers (MSI-H/dMMR), primary peritoneal cancer, breast cancer, Hodgkin lymphoma, gastric cancer, cervical cancer, primary mediastinal B-cell lymphoma (PMBCL), hepatocellular carcinoma (HCC), Merkel cell carcinoma (MCC), esophageal squamous cell cancer, cutaneous squamous cell carcinoma (cSCC), head and neck squamous cell cancer (HNSCC), bladder cancer, urothelial carcinoma, glioblastoma, melanoma, and T cell lymphomas.

Embodiment 40. The method of the combined or separate embodiments 35-39, wherein said administering is parenteral.

Embodiment 41. The method of embodiment 40, wherein said parenteral administering is selected from subcutaneous, intravenous, intra-arterial, intraperitoneal, intracardiac, intrathecal, intramuscular injection, and infusion.

Embodiment 42. Use of the conjugate of the combined or separate embodiments 1-32 or the composition of the combined or separate embodiments 33-34, in the treatment of a condition that is responsive to treatment with IFN-γ.

Embodiment 43. Use of the conjugate of the combined or separate embodiments 1-32 or the composition of the combined or separate embodiments 33-34, for use in the preparation of a medicament useful in the treatment of a condition that is responsive to treatment with IFN-γ.

Embodiment 44. A combination for use in treating a condition that is responsive to treatment with interferon-γ (IFN-γ), the combination comprising a therapeutically effective amount of the conjugate of the combined or separate embodiments 1-32 or the composition of the combined or separate embodiments 33-34; and a therapeutically effective amount of one or more of a Programmed Cell Death Protein 1 (PD-1) antagonist and a Programmed Cell Death Ligand 1 (PD-L1) antagonist.

Embodiment 45. A method for reducing heparin binding to interferon-γ (IFN-γ) by preparing an IFN-γ cysteine mutein conjugate in accordance with the combined or separate embodiments 1-32.

Embodiment 46. A method for reducing IFN-γ receptor-1 (IFNGR1) binding of an interferon-γ (IFN-γ) by preparing an IFN-γ cysteine mutein conjugate of the combined or separate embodiments 1-32.

Embodiment 47. A kit comprising a therapeutically effective amount of a conjugate of the combined or separate embodiments 1-32 or the composition of the combined or separate embodiments 33-34; accompanied by instructions for use in treating a condition that is responsive to treatment with IFN-γ.

1. A conjugate comprising interferon-gamma (IFN-γ) covalently attached to a water-soluble, non-peptidic polymer at a cysteine residue of the IFN-γ, wherein the IFN-γ is a mutein comprising a cysteine substitution or insertion (“IFN-γ cysteine mutein”).

2. The conjugate of claim 1, wherein the conjugate has a structure:
IFN-γ-s-x-POLY Formula I,
wherein IFN-γ is a cysteine mutein of IFN-γ, —S—, is a sulfur atom of the cysteine, X is a spacer moiety, and POLY is a water-soluble, non-peptidic polymer.

3. The conjugate of claim 1, having a structure: 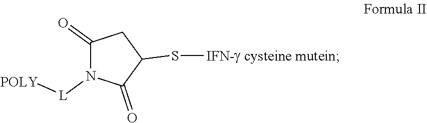
wherein the spacer moiety X comprises 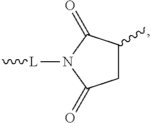
wherein L is a linker comprising one or more atoms selected from carbon, nitrogen, sulfur, oxygen, and combinations thereof,
POLY is the water-soluble, non-peptidic polymer; and
—S—is a sulfur atom of the cysteine.

4. The conjugate of claim 1, having a structure: 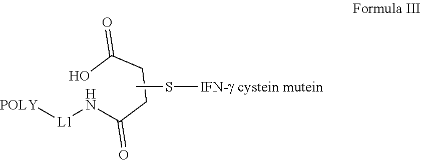
wherein spacer moiety X comprises 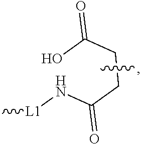
wherein L1 is a linker comprising one or more atoms selected from carbon, nitrogen, sulfur, oxygen, and combinations thereof,
POLY is the water-soluble, non-peptidic polymer; and
—S—is a sulfur atom of the cysteine.

5. The conjugate of claim 1, wherein the spacer or linker comprises one or more atoms selected from a carbon-carbon bond, an amide, a carbamate, an amine, an ether, and combinations thereof.

6. The conjugate of claim 3, wherein the linker L is ˜(CH₂)ₐ(O)_{b}[C(O)]_{c}(NH)_{d}(CH₂)ₑ(NH)_{f}[C(O)]_{g}(CH₂)ₕ˜, wherein: a is 0-10; b is 0, 1; c is 0, 1; d is 0, 1; e is 0-10; f is 0, 1; g is 0, 1; and h is 0-10, wherein at least one of a, b, c, d, e, f, g, and h is a positive integer.

7. The conjugate of claim 6, where f is 1, g is 1 and h is 2.

8. The conjugate of claim 7, where a, b, c, d, and e are zero.

9. The conjugate of claim 7, wherein a is 3, b is 0, c is 1, d is 1 and e is 2.

10. (canceled)

11. The conjugate of claim 10, wherein POLY is branched and the branched POLY comprises from about 2 to about 10 polymer arms.

12. The conjugate of claim 1, wherein the water-soluble, non-peptidic polymer is a poly(alkylene oxide).

13. The conjugate of claim 12, wherein the poly(alkylene oxide) is a poly(ethylene oxide).

14. The conjugate of claim 13, wherein POLY comprises —(CH₂CH₂O)ₙ—Y or —(OCH₂CH₂)ₙ—Y, wherein Y is selected from a lower alkyl or hydroxyl; and
n is an integer ranging from about 45 to about 1818.

15. The conjugate of claim 14, wherein the lower alkyl is methyl.

16. The conjugate of claim 13, wherein POLY comprises a structure: 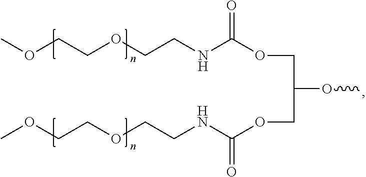
wherein each n is independently an integer ranging from about 45 to about 1818.

17. The conjugate of claim 1, wherein POLY has a weight average molecular weight of from about 2,000 daltons to about 80,000 daltons, a weight average molecular weight of from about 2,000 daltons to about 40,000 daltons, or a weight average molecular weight of from about 10,000 daltons to about 40,000 daltons.

18.-19. (canceled)

20. The conjugate of claim 1, wherein the IFN-γ cysteine mutein has a sequence having at least 95% sequence identity to a sequence set forth in SEQ ID NO: 4, SEQ ID NO: 5, or SEQ ID NO:6.

21. The conjugate of claim 1, wherein the IFN-γ cysteine mutein has a sequence having at least 95% sequence identity to SEQ ID NO:1, SEQ ID NO:2, SEQ ID NO:3 or SEQ ID NO: 7 and comprises a cysteine substitution or insertion.

22. The conjugate of claim 21, wherein the IFN-γ cysteine mutein sequence includes a cysteine residue substituted for at least one amino acid selected from the group consisting of the serine at position 66, the asparagine at position 98, and the methionine at position 135 of SEQ ID NO:3 or SEQ ID NO:7.

23. The conjugate of claim 1, wherein the cysteine substitution or insertion is located within the interferon gamma receptor 1 (IFNGR1) binding region of the IFN-γ mutein, is located within 1-10 amino acids from either end of the IFNGR1 binding region of the IFN-γ cysteine mutein, or is located at the C-terminus of the IFN-γ mutein.

24.-25. (canceled)

26. The conjugate of claim 1, wherein the conjugate has a structure selected from:

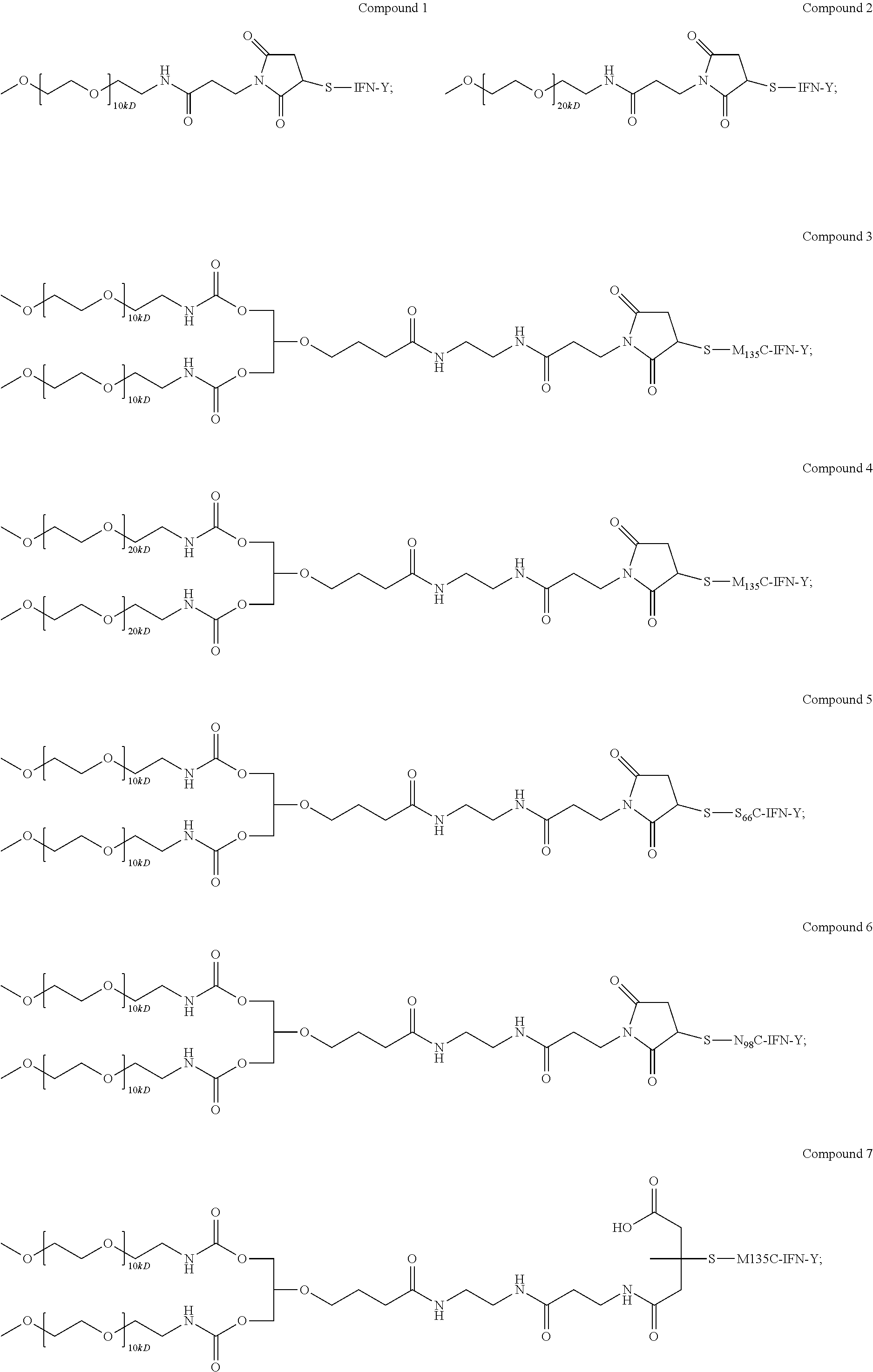

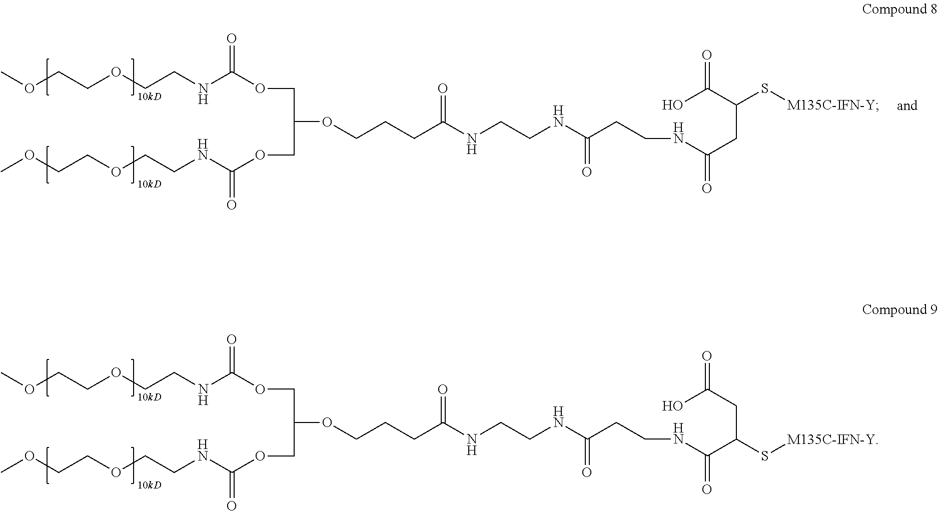

27. The conjugate of claim 1, having an EC50 value (ng/mL, human PMBCs pSTAT1) that is reduced by at least about 3-fold when compared to the EC50 value (ng/mL, human PMBCs pSTAT1) of the non-polymer modified IFN-γ cysteine mutein and/or unmodified IFN-γ, or at least about 3.5-fold, or at least about 4-fold, or at least about 4.5-fold, or at least about 5-fold, or at least about 5.5-fold, or at least about 6-fold, or at least about 6.5-fold, or at least about 7-fold, or at least about 7.5-fold, or at least about 8-fold, or at least about 8.5-fold, or at least about 9-fold, or at least about 9.5-fold, or at least about 10-fold when compared to the EC50 value (ng/mL, human PMBCs pSTAT1) of the non-polymer modified IFN-γ cysteine mutein and/or unmodified IFN-γ.

28. The conjugate of claim 1, wherein the conjugate exhibits a reduction in major histocompatibility complex class I (MHCI) induction of no more than about 3-fold as measured by its EC50 value (ng/mL, HT-29 MHCI) when compared to the MHCI induction as measured by EC50 value (ng/mL, HT-29 MHCI) of the non-polymer modified IFN-γ cysteine mutein and/or unmodified IFN-γ, or no more than about 3.5-fold, or no more than about 4-fold, or no more than about 4.5-fold, or no more than about 5-fold when compared to the MHCI induction as measured by EC50 value (ng/mL, HT-29 MHCI) of the non-polymer modified IFN-γ cysteine mutein and/or unmodified IFN-γ.

29. The conjugate of claim 1, wherein the conjugate exhibits a reduction in major histocompatibility complex class II (MHCII) induction of no more than about 3-fold as measured by its EC50 value (ng/mL, HT-29 MHCII) when compared to the MHCI induction as measured by EC50 value (ng/mL, HT-29 MHCII) of the non-polymer modified IFN-γ cysteine mutein and/or unmodified IFN-γ, or no more than about 3.5-fold, or no more than about 4-fold, or no more than about 4.5-fold, or no more than about 5-fold when compared to the MHCI induction as measured by EC50 value (ng/mL, HT-29 MHCII) of the non-polymer modified IFN-γ cysteine mutein and/or unmodified IFN-γ.

30. The conjugate of claim 1, exhibiting a reduction in IFNGR1 binding (K_{D}) of at least about 3% when compared to IFNGR1 binding (K_{D}) of the non-polymer modified IFN-γ cysteine mutein and/or unmodified IFN-γ, or at least about 4%, or at least about 5%, or at least about 6%, or at least about 7%, or at least about 8%, or at least about 9%, or at least about 10%.

31. The conjugate of claim 1, having a decrease in heparin binding (Kᵢ) of at least about 1% when compared to heparin binding (Kᵢ) of the non-polymer modified IFN-γ cysteine mutein and/or unmodified IFN-γ, or at least about 2%, or at least about 3%, or at least about 4%, or at least about 5%.

32. The conjugate of claim 1, having a reduction in heparin binding (Kᵢ, nM) of at least about a 1 fold when compared to the heparin binding (Kᵢ, nM) of the non-polymer modified IFN-γ cysteine mutein and/or unmodified IFN-γ, or at least about 1.5-fold.

33. A composition comprising a conjugate of claim 1 and a pharmaceutically acceptable excipient.

34. The composition of claim 33, wherein no more than about 15 mole percent of conjugates comprised in the composition have the following ring-closed structure:

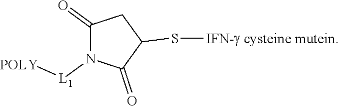

35. A method for treating a subject having a disease that is responsive to treatment with IFN-γ comprising:
administering to the subject a therapeutically effective amount of the conjugate of claim 1.

36. The method of claim 35, wherein the disease is a cancer.

37.-38. (canceled)

39. The method of claim 36, wherein the cancer is selected from small cell lung cancer (SCLC), non-small cell lung cancer (NSCLC), adrenocortical carcinoma, myxoid/round cell liposarcoma, synovial sarcoma, gliosarcoma, fallopian tube cancer, ovarian cancer, renal cell carcinoma (RCC), colorectal cancer, microsatellite instability-high cancers (MSI-H/dMMR), primary peritoneal cancer, breast cancer, Hodgkin lymphoma, gastric cancer, cervical cancer, primary mediastinal B-cell lymphoma (PMBCL), hepatocellular carcinoma (HCC), Merkel cell carcinoma (MCC), esophageal squamous cell cancer, cutaneous squamous cell carcinoma (cSCC), head and neck squamous cell cancer (HNSCC), bladder cancer, urothelial carcinoma, glioblastoma, melanoma, and T cell lymphomas.

40. The method of any one of claim 35, wherein said administering is parenteral, selected from subcutaneous, intravenous, intra-arterial, intraperitoneal, intracardiac, intrathecal, intramuscular injection, and infusion.

41.-43. (canceled)

44. A combination for use in treating a condition that is responsive to treatment with interferon-γ (IFN-γ), the combination comprising:
a therapeutically effective amount of the conjugate of claim 1; and
a therapeutically effective amount of one or more of a Programmed Cell Death Protein 1 (PD-1) antagonist and a Programmed Cell Death Ligand 1 (PD-L1) antagonist.

45.-46. (canceled)

47. A kit comprising:
a therapeutically effective amount of the conjugate of claim 1; accompanied by instructions for use in treating a condition that is responsive to treatment with IFN-γ.

48. The conjugate of claim 4, wherein the linker L1 is ˜(CH₂)ₐ(O)_{b}[C(O)]_{c}(NH)_{d}(CH₂)ₑ(NH)_{f}[C(O)]_{g}(CH₂)ₕ˜, wherein: a is 0-10; b is 0, 1; c is 0, 1; d is 0, 1; e is 0-10; f is 0, 1; g is 0, 1; and h is 0-10, wherein at least one of a, b, c, d, e, f, g, and h is a positive integer.

49. The conjugate of claim 48, where f is 1, g is 1 and h is 2.

50. The conjugate of claim 48, where a, b, c, d, and e are zero.

51. The conjugate of claim 48, wherein a is 3, b is 0, c is 1, d is 1 and e is 2.
